(19) United States
(12) Patent Application Publication
(10) Pub. No.: US 2017/0369904 A1
(43) Pub. Date: Dec. 28, 2017

(54) METHODS FOR INCREASING CELL CULTURE TRANSFECTION EFFICIENCY AND CELLULAR REPROGRAMMING

(71) Applicant: Xcell Biosciences, Inc., San Francisco, CA (US)

(72) Inventors: James LIM, Oakland, CA (US); Luke CASSEREAU, Emeryville, CA (US)

(21) Appl. No.: 15/629,240

(22) Filed: Jun. 21, 2017

Related U.S. Application Data
(60) Provisional application No. 62/405,725, filed on Oct. 7, 2016.
(60) Provisional application No. 62/362,214, filed on Jul. 14, 2016.
(60) Provisional application No. 62/353,435, filed on Jun. 22, 2016.

(51) Int. Cl. C12N 15/87; C12N 5/0783; C12N 15/88; C12N 5/077; C12N 5/074

(52) U.S. Cl. CPC C12N 15/87; C12N 5/0656; C12N 5/0696; C12N 15/88; C12N 5/0638; C12N 2500/90; C12N 2510/00; C12N 2527/00; C12N 2500/02

(57) ABSTRACT

The present invention describes a method for increasing transfection efficiency of cells. The present invention further provides a method for increasing the efficiency of stem cell reprogramming.

### CROSS REFERENCE

This application claims the benefit of U.S. Provisional Application No. 62/405,725, filed Oct. 7, 2016; U.S. Provisional Application No. 62/362,214, filed Jul. 14, 2016; and U.S. Provisional Application No. 62/353,435, filed Jun. 22, 2016, each of which is incorporated herein by reference in its entirety.

### BACKGROUND

Transfection methods can be used to introduce nucleic acids into cultured cells. Transfection methods have become a mainstay of studies related to gene regulation, gene function, molecular therapy, signal transduction, drug screening, and gene therapy. Transfection efficiency can vary based on cell culture conditions, cell type, cell viability and health, cell confluency, cell culture media, serum, and type of nucleic acid used for transfection. A method for increasing cell culture transfection efficiency could lead to improvements in genetic manipulation of cells and, in turn, future therapeutic studies.

Stem cell reprogramming is a cell culture technique that can be used in the field of regenerative medicine. Induced pluripotent stem cells (iPSCs) can be used to replace those cells lost due to damage or disease in afflicted patients. Current methods of stem cell reprogramming can be inefficient and time-consuming. Thus, a method for increasing stem cell reprogramming efficiency could lead to improvements in future therapeutic studies.

### SUMMARY OF THE INVENTION

In some embodiments, the invention provides a method for increasing transfection efficiency of a nucleic acid that is introduced into a cell, the method comprising culturing the cell in a hypoxic condition and a positive pressure condition, wherein culturing the cell in the hypoxic condition and the positive pressure condition increases expression of a polypeptide encoded by the nucleic acid that is introduced into the cell as compared to expression of the polypeptide encoded by a nucleic acid that is introduced into a cell that is cultured in the absence of the hypoxic condition and the positive pressure condition.

In some embodiments, the invention provides a method for reprogramming a cell, the method comprising culturing the cell in a hypoxic condition and a positive pressure condition, wherein the cell exhibits a rate of reprogramming that is higher than the rate of reprogramming of a cell cultured in the absence of the hypoxic condition and the positive pressure condition.

### INCORPORATION BY REFERENCE

Each patent, publication, and non-patent literature cited in the application is hereby incorporated by reference in its entirety as if each was incorporated by reference individually.

### BRIEF DESCRIPTION OF THE FIGURES

FIG. 1 depicts an illustrative transfection workflow of the invention

FIG. 2 depicts an experimental procedure for comparison of electroporation versus a method described herein.

FIG. 3 depicts the results of transfection of cells using a method described herein.

FIG. 4 depicts the results of transfection of cells using a method described herein.

FIG. 5 depicts the results of transfection of PBMCs from two different donors using a method described herein.

FIG. 6 is an illustrative computer system to be used with a method described herein.

FIG. 7 depicts a workflow that can be used for the reprogramming of stem cells.

FIG. 8 illustrates the average fold increase in stem cell colony number using a method described herein.

FIG. 9 illustrates the distribution of stem cell colony area using a method described herein.

FIG. 10 depicts the cell morphology of cells cultured using a method described herein.

FIG. 11 provides the reprogramming kinetics of stem cells cultured using a method described herein.

FIG. 12 depicts cardiomyocyte differentiation using a method described herein.

FIG. 13 depicts the effect of conditions described herein on stem cell pluripotency and differentiation.

FIG. 14 depicts the effect of conditions described herein on stem cell differentiation markers.

FIG. 15 depicts immunofluorescence of stem cell markers using a method described herein.

FIG. 16 illustrates the average colony area size of differentiated stem cells using a method described herein.

FIG. 17 shows the gene expression profile of a population of cells as a function of oxygen concentration and pressure as compared to a standard cell culture incubator.

FIG. 18 depicts the change in transfection efficiency with changes in oxygen and pressure levels.

FIG. 19 depicts the change in transfection efficiency with changes in oxygen and pressure levels.

FIG. 20 provides a workflow for measuring transfection efficiency using a method disclosed herein.

FIG. 21 shows the change in transfection efficiency via GFP expression with changes in oxygen and pressure levels.

FIG. 22 shows the quantification of transfection efficiency via GFP expression of FIG. 21 with changes in oxygen and pressure levels.

FIG. 23 shows a comparison between the transfection of CD8+ cells enriched from PBMCs and PBMCs with a GFP plasmid using a method described herein.

FIG. 24 shows the quantification of the results of FIG. 23.

FIG. 25 shows that the GFP-transfected CD8+ cells cultured under hypoxic and high pressure conditions developed more multicellular clusters than did cells grown at standard incubator conditions.

FIG. 26 shows the percent GFP in the multicellular clusters in cells grown under hypoxic and high pressure conditions compared to cells grown under standard incubator conditions.

FIG. 27 shows the quantification of the results of FIG. 26.

FIG. 28 depicts that when a CRISPR/Cas9 system was used to knockout CTLA4, and knock-in GFP using homology-directed repair, the transfection efficiency of the CRISPR/Cas9 was higher in the cells grown under hypoxic and high pressure conditions than in standard incubator conditions.

FIG. 29 shows that the cells grown under hypoxic and high pressure conditions developed a higher percentage of GFP-positive multicellular clusters than the cells grown at standard culture conditions.

FIG. 30 shows that the proliferation of the CD8+ cells grown under hypoxic and high pressure conditions was enriched over the cells grown under standard incubator conditions.

FIG. 31 depicts a limited dilution assay workflow to assess GFP-positive colonies using the CRISPR/Cas9 system.

FIG. 32 shows genome editing of the CD8-positive T-cells as indicated by the GFP signal.

FIG. 33 shows that a combination of low oxygen and high pressure enhances ectoderm commitment in defined medium, while causing changes in colony morphology to more mesoderm-like morphology.

FIG. 34 shows the change in various stem cell markers upon incubation of cells using a method disclosed herein.

FIG. 35 show that different combinations of tumor (disease) extracellular matrix (ECM), low oxygen, and high pressure can alter the gene expression of EGFR and other metabolic regulators in DU145 (prostate cancer) and Panc10 (pancreatic cell lines).

FIG. 36 shows that PDL1 expression increased in ARV7-positive, 22RV1 prostate cancer cells during low oxygen and high pressure culturing conditions.

FIG. 37 (top panel) provides a western blot showing increased PDL1 protein expression under various conditions of high pressure and hypoxia in both DU145 and 22Rv1 prostate cancer cells. The bottom panel of FIG. 37 provides a quantification of the western blot results normalized to actin.

FIG. 38 shows identification of pressure and oxygen sensitive gene expression signatures in various cell lines.

FIG. 39 shows a workflow of taking a biopsy culture taken from a patient having prostate cancer.

FIG. 40 shows thst prostate cancer cells were able to form an organoid after two weeks of culture under high pressure and low oxygen conditions.

FIG. 41 shows a workflow of taking an apheresis culture taken from a patient having prostate cancer.

FIG. 42 shows the mutations found using the COSMIC database from pancreatic ductal adenocarcinoma (PDAC) and circulating tumor cells (CTC) using whole exome sequencing (top panels).

FIG. 43 shows that there was increased ex vivo expansion of primary cells under low oxygen and high pressure.

FIG. 44 shows that there was increased ex vivo expansion of primary cells under low oxygen and high pressure.

FIG. 45 shows the effect that various oxygen and pressure conditions had on the gene expression of immunotherapeutic targets in donor PBMCs.

FIG. 46 shows the results of the ex vivo culture and expansion of tumor-infiltrating lymphocytes (TILs) enriched from renal cell carcinoma tumors using high pressure and low conditions.

FIG. 47 shows that hypoxic and high pressure conditions can lead to greater enrichment of CD8+ cells from fresh blood samples than culture under standard incubator conditions.

FIG. 48 shows an expanded culture time, which indicated that the culture under hypoxic and high pressure conditions generates more CD8+ cells from whole blood than culture under standard conditions.

FIG. 49 shows induction of neural precursor markers, PAX6 and NESTIN, in iPSCs after two weeks in culture under 5% O₂ and 2 PSI in stem cell maintenance media.

FIG. 50 shows ex vivo cultures of pancreatic ductal adenocarcinoma colonies from a fine-needle aspirate.

FIG. 51 shows ex vivo cultures of pancreatic ductal adenocarcinoma colonies from a fine-needle aspirate.

FIG. 52 shows ex vivo cultures of pancreatic ductal adenocarcinoma colonies from a fine-needle aspirate.

FIG. 53 shows the transfection of human dermal fibroblasts using electroporation of a GFP plasmid.

FIG. 54 shows the transfection of PBMCs using electroporation of a GFP plasmid.

FIG. 55 shows the transfection of activated CD8+ T-cells using electroporation of a GFP plasmid.

FIG. 56 shows the post-transfection effects of CD8+ T-cells using low oxygen and high pressure conditions.

FIG. 57 shows a heatmap of the effect on pressure-sensitive genes under various experimental conditions.

FIG. 58 shows a heatmap of the effect on oxygen-sensitive genes under various experimental conditions.

FIG. 59 is a molecular confirmation of a genome editing experiment.

FIG. 60 is a molecular confirmation of a genome editing experiment.

### DETAILED DESCRIPTION

Transfection.

A method described herein can be used to increase, for example, transfection and transduction efficiency in cells. Transduction can be used, for example, to introduce a viral vector in a cell. Viral nucleic acid delivery systems can use recombinant viruses to deliver nucleic acids for gene therapy. Non-limiting examples of viruses that can be used to deliver nucleic acids include retrovirus, adenovirus, herpes simplex virus, adeno-associated virus, vesicular stomatitis virus, reovirus, vaccinia, pox virus, lentivirus, and measles virus.

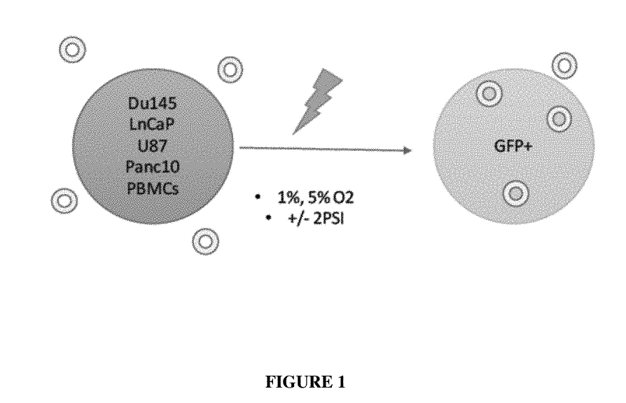

Transfection methods that can be used with methods of the invention include, for example, lipofection, electroporation, calcium phosphate transfection, chemical transfection, polymer transfection, gene gun, magnetofection, or sonoporation. FIG. 1 depicts an illustrative transfection workflow of the invention. FIG. 1 shows the transfection of, for example, DU145 (human prostate cancer), LnCaP (androgen-sensitive human prostate adenocarcinoma), U87 (human primary glioblastoma), PANC10 (pancreatic adenocarcinoma), or PBMCs (peripheral blood mononuclear cells) with a GFP (green fluorescent protein) plasmid. The cells can be cultured in hypoxic conditions, for example, at 1% or 5% oxygen, and at conditions that are about 2 PSI greater or less than normal pressure conditions. The transfection allows introduction of the GFP-expressing plasmid into the cell.

Viral nucleic acid delivery methods can use recombinant viruses for nucleic acid transfer. Non-viral nucleic acid delivery can comprise injecting naked DNA or RNA, use of carriers including lipid carriers, polymer carriers, chemical carriers and biological carriers such as biologic membranes, bacteria, and virus-like particles, and physical/mechanical approaches. A combination of viral and non-viral nucleic acid delivery methods can be used for efficient gene therapy.

Non-viral nucleic acid transfer can include injection of naked nucleic acid, for example, nucleic acid that is not protected or devoid of a carrier. Hydrodynamic injection methods can increase the targeting ability of naked nucleic acids.

Non-viral nucleic acid delivery systems can include chemical carriers. These systems can include lipoplexes, polyplexes, dendrimers, and inorganic nanoparticles. A lipoplex is a complex of a lipid and a nucleic-acid that protects the nucleic acid from degradation and facilitates entry into cells, and can be prepared from neutral, anionic, or cationic lipids. Lipoplexes can enter cells by endocytosis, and release the nucleic acid contents into the cytoplasm. A polyplex is a complex of a polymer and a nucleic acid, and are prepared from cationic polymers that facilitate assembly by ionic interactions between nucleic acids and polymers. Uptake of polyplexes into cells can occur by endocytosis. Inside the cells, polyplexes require co-transfected endosomal rupture agents such as inactivated adenovirus, for the release of the polyplex particle from the endocytic vesicle. Examples of polymeric carriers include polyethyleneimine, chitosan, poly(beta-amino esters) and polyphosphoramidate. Dendrimers can be constructed to have a positively-charged surface and/or carry functional groups that aid temporary association of the dendrimer with nucleic acids. These dendrimer-nucleic acid complexes can be used for gene therapy. The dendrimer-nucleic acid complex can enter the cell by endocytosis. Nanoparticles prepared from inorganic material can be used for nucleic acid delivery. Examples of inorganic material can include gold, silica/silicate, silver, iron oxide, and calcium phosphate. Inorganic nanoparticles with a size of less than 100 nm can be used to encapsulate nucleic acids efficiently. The nanoparticles can be taken up by the cell via endocytosis, and the nucleic acid can be released from the endosome without degradation. Nanoparticles based on quantum dots can be prepared and offers the use of a stable fluorescence marker coupled with gene therapy. Organically modified silica or silicate can be used to target nucleic acids to specific cells in an organism.

Non-viral nucleic acid delivery systems can include biological methods including bactofection, biological liposomes, and virus-like particles (VLPs). The bactofection method comprises using attenuated bacteria to deliver nucleic acids to a cell. Biological liposomes, such as erythrocyte ghosts and secretion exosomes, are derived from the subject receiving gene therapy to avoid an immune response. Virus-like particles (VLP) or empty viral particles are produced by transfecting cells with only the structural genes of a virus and harvesting the empty particles. The empty particles are loaded with nucleic acids to be transfected for gene therapy.

Examples of physical methods of transfection include electroporation, gene gun, sonoporation, and magnetofection. The electroporation method uses short high-voltage pulses to transfer nucleic acid across the cell membrane. These pulses can lead to formation of temporary pores in the cell membrane, thereby allowing nucleic acid to enter the cell. Electroporation can be efficient for a broad range of cells. Electron-avalanche transfection is a type of electroporation method that uses very short, for example, microsecond, pulses of high-voltage plasma discharge for increasing efficiency of nucleic acid delivery. The gene gun method utilizes nucleic acid-coated gold particles that are shot into the cell using high-pressure gas. Force generated by the gene gun allows penetration of nucleic acid into the cells, while the gold is left behind on a stopping disk. The sonoporation method uses ultrasonic frequencies to modify permeability of cell membrane. Change in permeability allows uptake of nucleic acid into cells. The magnetofection method uses a magnetic field to enhance nucleic acid uptake. In this method, nucleic acid is complexed with magnetic particles. A magnetic field is used to concentrate the nucleic acid complex and bring them in contact with cells.

Non-limiting examples of viruses that can be used to deliver nucleic acids include retrovirus, adenovirus, herpes simplex virus, adeno-associated virus, vesicular stomatitis virus, reovirus, vaccinia, pox virus, and measles virus.

Non-limiting examples of retroviral vectors include Moloney murine leukemia viral (MMLV) vectors, HIV-based viral vectors, gammaretroviral vectors, C-type retroviral vectors, and lentiviral vectors. Lentivirus is a subclass of retrovirus. While some retroviruses can infect only dividing cells, lentiviruses can infect and integrate into the genome of actively dividing cells and non-dividing cells.

An adenovirus is a non-enveloped virus with a linear double-stranded genome. Adenoviruses can enter host cells using interactions between viral surface proteins and host cell receptors that lead to endocytosis of the adenovirus particle. Once inside the host cell cytoplasm, the adenovirus particle is released by the degradation of the endosome. Using cellular microtubules, the adenovirus particle gains entry into the host cell nucleus, where adenoviral DNA is released. Inside the host cell nucleus, the adenoviral DNA is transcribed and translated, without integrating into the host cell genome.

Herpes simplex virus (HSV)-based vectors can be used in the disclosure. The HSV is an enveloped virus with a linear double-stranded DNA genome. Interactions between surface proteins on the host cell and HSV lead to pore formation in the host cell membrane. These pores allow HSV to enter the host cell cytoplasm, and once inside the host cell, the HSV uses the nuclear entry pore to enter the host cell nucleus where HSV DNA is released. HSV can persist in host cells in a state of latency. Herpes simplex virus 1 and 2 (HSV-1 and HSV-2), also known as human herpes virus 1 and 2 (HHV-1 and HHV-2), are members of the herpes virus family.

Alphavirus-based vectors can be used to deliver nucleic acids. Examples of alphavirus-based vectors include vectors derived from semliki forest virus and sindbis virus.

Pox/vaccinia-based vectors such as orthopox or avipox vectors can be used in the present invention. Pox virus is a double stranded DNA virus that can infect diving and non-dividing cells. Pox viral genome can accommodate up to 25 kb transgenic sequence. Multiple genes can be delivered using a single vaccinia viral vector.

Adeno-associated virus (AAV) is a small, non-enveloped virus that belongs to the Parvoviridae family. The AAV genome is a linear single-stranded DNA molecule of about 4,800 nucleotides. The AAV DNA comprises two inverted terminal repeats (ITRs) at both ends of the genome and two sets of open reading frames. The ITRs serve as origins of replication for the viral DNA and as integration elements. The open reading frames encode for the Rep (non-structural replication) and Cap (structural capsid) proteins. AAV can infect dividing cells and quiescent cells. AAV can be engineered for use as a gene therapy vector by substituting the coding sequence for both AAV genes with a transgene (transferred nucleic acid) to be delivered to a cell. The substitution eliminates immunologic or toxic side effects due to expression of viral genes. The transgene can be placed between the two ITRs (145 bp) on the AAV DNA molecule.

A pseudotyped virus can be used for the delivery of nucleic acids. Pseudotyping involves substitution of endogenous envelope proteins of the virus by envelope proteins from other viruses or chimeric proteins. The foreign envelope proteins can confer a change in host tropism or alter stability of the virus. An example of a pseudotyped virus useful for gene therapy includes vesicular stomatitis virus G-pseudotyped lentivirus (VSV G-pseudotyped lentivirus) that is produced by coating the lentivirus with the envelope G-protein from Vesicular stomatitis virus. VSV G-pseudotyped lentivirus can transduce almost all mammalian cell types.

A hybrid vector having properties of two or more vectors can be used for nucleic acid delivery to a host cell. Hybrid vectors can be engineered to reduce toxicity or improve therapeutic transgene expression in target cells. Non-limiting examples of hybrid vectors include AAV/adenovirus hybrid vectors, AAV/phage hybrid vectors, and retrovirus/adenovirus hybrid vectors.

A viral vector can be replication-competent. A replication-competent vector contains all the genes necessary for replication, making the genome lengthier than replication-defective viral vectors. A viral vector can be replication-defective, wherein the coding region for the genes essential for replication and packaging are deleted or replaced with other genes. Replication-defective viruses can transduce host cells and transfer the genetic material, but do not replicate. A helper virus can be supplied to help a replication-defective virus replicate.

A viral vector can be derived from any source, for example, humans, non-human primates, dogs, fowl, mouse, cat, sheep, and pig.

The nucleic acid of the disclosure can be generated using any method. The nucleic acid can be synthetic, recombinant, isolated, and/or purified.

A vector of the present disclosure can comprise one or more types of nucleic acids. The nucleic acids can include DNA or RNA. RNA nucleic acids can include a transcript of a gene of interest. DNA nucleic acids can include the gene of interest, promoter sequences, untranslated regions, and termination sequences. A combination of DNA and RNA can be used. The nucleic acids can be double-stranded or single-stranded. The nucleic acid can include non-natural or altered nucleotides.

A vector of the disclosure can comprise nucleic acids encoding a selectable marker. The selectable marker can be positive, negative or bifunctional. The selectable marker can be an antibiotic-resistance gene. Examples of antibiotic resistance genes include markers conferring resistance to kanamycin, gentamicin, ampicillin, chloramphenicol, tetracycline, doxycycline, hygromycin, puromycin, zeomycin, or blasticidin. The selectable marker can allow imaging of the host cells, for example, a fluorescent protein. Examples of imaging marker genes include GFP, eGFP, RFP, CFP, YFP, dsRed, Venus, mCherry, mTomato, and mOrange.

The transfection can be a stable or transient transfection. The transfection can be used to transfect DNA plasmids, RNA, siRNA, shRNA, or any nucleic acid. The plasmids can encode, for example, green fluorescent protein (GFP), selectable markers, and other proteins of interest. The selectable markers can provide resistance to, for example, G418, hygromycin B, puromycin, and blasticidin.

A Clustered Regularly Interspaced Short Palindromic Repeats (CRISPR)—CRISPR associated (Cas) (CRISPR-Cas) system can be used to modify a target or deliver a nucleic acid of the disclosure. The CRIPSR-Cas system is a targeted genome-editing system comprising a Cas nuclease that is guided to specific DNA sequences, for example, a genomic locus in a subject, by a guide RNA molecule. The Cas nuclease can modify the genomic locus, for example, by cleaving the genomic locus, thus generating mutations that result in loss of function of the target sequence. The Cas nuclease can also modify the genomic locus, for example, by cleaving the genomic locus, and adding a transgene, for example, a therapeutic nucleic acid of the disclosure. The CRIPSR/Cas system can be used in conjunction with other nucleic acid delivery methods such as viral vectors and non-viral methods as described herein.

A CRISPR interference (CRISPRi) system can be used to modify the expression of a target of the disclosure. The CRISPRi system is a targeted gene regulatory system comprising a nuclease deficient Cas enzyme fused to a transcriptional regulatory domain that is guided to specific DNA sequences, for example, a genomic locus in a subject, by a guide RNA molecule. The Cas/regulator fusion protein can occupy the genomic locus and induce, for example, transcriptional repression of the target gene through the function of a negative regulatory domain fused to the Cas protein. The CRISPRi system can be used in conjunction with other nucleic acid delivery methods such as viral vectors and non-viral methods as described herein.

A method of the invention can increase the transfection or transduction efficiency by, for example, about 2-fold, about 3-fold, about 4-fold, about 5-fold, about 6-fold, about 7-fold, about 8-fold, about 9-fold, about 10-fold, about 12-fold, about 14-fold, about 16-fold, about 18-fold, about 20-fold, about 25-fold, about 30-fold, about 35-fold, about 40-fold, about 45-fold, about 50-fold, about 60-fold, about 70-fold, about 80-fold, about 90-fold, or about 100-fold.

In some embodiments, a hypoxic or positive pressure condition is applied to a cell prior to transfection. In some embodiments, a hypoxic or positive pressure condition is applied to a cell after transfection. A method described herein can comprise a conditioning step, where the conditioning step is for 24-48 hours and comprises culturing the cell to be transfected in a hypoxic or high pressure condition prior to the transfection. A method described herein can comprise a recovery period, where the recovery period comprises culturing a cell post-transfection in a hypoxic or positive pressure condition. In some embodiments, a transfection method described herein comprises a conditioning step, where the conditioning step comprises culturing the cell prior to transfection in a hypoxic or positive pressure condition for 24-48 hours. In some embodiments, a transfection method described herein comprises a recovery period, where the recovery period comprises culturing the cell after transfection in a hypoxic or positive pressure condition. In some embodiments, a transfection method described herein comprises both a conditioning step and a recovery period.

In some embodiments, a conditioning step prior to transfection can use moderate oxygen and moderate pressure levels to efficiently propagate cells while maintaining, for example, pluripotency. The oxygen levels can vary from about 5% to about 15%. Pressure levels can vary from about 0.1 PSI to about 2 PSI.

In some embodiments, a recovery phase after a transfection can use low oxygen and high pressure levels to increase transfection and recovery of cells by increasing cell viability. The oxygen levels can vary from about 0.1% to about 2%. Pressure levels can vary from about 2 PSI to about 5 PSI.

In some embodiments, positive pressure is used to increase transfection efficiency. In some embodiments, hypoxia is used to increase transfection efficiency. In some embodiments, hypoxia and positive pressure are used to increase transfection efficiency.

### Stem Cells.

A method disclosed herein can be used to reprogram, for example, fibroblasts to pluripotent stem cells. A method disclosed herein can, for example, increase the efficiency and increase the rate of cell reprogramming. A method disclosed herein can further increase, for example, the number and size of stem cell colonies that form as a result of the reprogramming protocol. The cells can be reprogrammed into, for example, totipotent, pluripotent, multipotent, oligopotent, or unipotent stem cells.

Reprogramming of cells into pluripotent stem cells can be enhanced by, for example, culturing the cells under hypoxic and positive pressure conditions. The cells can be reprogrammed by transfecting cells with, for example, an RNA replicon vector encoding several stem cell transformation factors. The stem cell transformation factors can include, for example, Oct4, Sox2, KLF-4, GLIS1, and c-MYC. Additional stem cell transformation factors include, for example, Nanog and Lin28. After transfection of the cells with the reprogramming factors, the cells can be maintained in media designed to differentiate and maintain stem cell populations. The cells can be grown under hypoxic and high pressure conditions as disclosed herein to induce differentiation of the cells.

Adult stem cells can be found in many organs and tissues including, for example, brain, bone marrow, peripheral blood, blood vessels, skeletal muscle, skin, teeth, heart, gut, liver, ovarian epithelium, and testis. The stem cells can reside in stem cell niches within the various areas of the body. In many tissues, some types of stem cells are pericytes, which are cells that compose the outermost layer of small blood vessels. Stem cells may remain quiescent non-dividing for long periods of time until they are activated by a normal need for more cells to maintain tissues, or by disease or tissue injury.

Markers that can be used to identify iPSCs include, for example, SSEA-3, SSEA-4, TRA-1-60, TRA-1-81, TRA-2-49/6E, Nanog, Oct3/4, Sox2, GDF3, REX1, FGF4, ESG1, DPPA2, DPPA4, and hTERT.

The iPSCs can be induced to differentiate into, for example, neuronal cells, hippocampal progenitors, dentate granule cell neurons, MGE progenitors, cortical interneurons, dorsal cortical progenitors, excitatory cortical neurons, glial progenitors, astrocytes, neural crest stem cells, dopaminergic neurons, oligodendrocytes, dopaminergic neurons, hematopoietic cells, B-cells, T-cells, NK cells, granulocytes, monocytes, macrophages, erythrocytes, megakaryocytes, platelets, cardiomyocytes, hepatocytes, skeletal muscle cells, adipocytes, pancreatic beta-cells, or cells from the ectoderm, mesoderm, or endoderm.

The stem cells obtained using a method disclosed herein can be cultured on, for example, a gelatin-coated culture dish. The cells can be in cultured in medium containing inactivated mouse embryonic fibroblast (MEF) medium, basic FGF solution, pluripotent culture medium, leukemia inhibitory factor, and a collagenase solution. The stem cells can additionally be grown over a layer of feeder cells, which can be, for example, MEFs, JK1 cells, or SNL 76/7 cells.

Expression markers that can be measured to assess the differentiation or gene expression profile of an initial cell culture to iPSCS can include, for example, IGF1, CTNNB1, AXIN1, KAT2A, CD4, CXCL12, FZD9, CD44, ACTC1, JAG1, BMP1, FZD2, IL6ST, FZD7, LIFR, SMAD4, DVL1, CTNNA1, FGFR1, WNT1, PPARG, COL1A1, FGF1, GLL, DNMT3B, PSEN1, ALDH1A1, JUND, SDAD1, NCSTN, FZD6, TCF7, NOTCH1, APC, RB1, NUMB, CREBBP, GATA6, PSEN2, HDAC2, CCND1, CCNE1, EP300, Notch2, MME, GLI2, BTRC, STAT3, PPARD, Notch3, Notch4, GLI3, CDC42, CCNA2, ISL1, BMP2, PAX6, S100B, CD3D, FZD5, Nanog, CDH1, Sox1, DLL1, CCND2, SMO, COL2AI, LIFR, or COX2.

### Plant Cells.

A method disclosed herein can be used to genetically engineer or to reprogram plant cells. A method disclosed herein can be used to create plant cells with a particular genotype that alters the cell's ability to produce a specific molecule or that results in a specific phenotype. Some embodiments of the invention comprise modulating local pressure and oxygen conditions during transformation of plant cells.

A method disclosed herein can be applied to any type of plant cell or tissue. Plant cells or tissues used in the invention can include roots, leaves, monocotyledons such as cotton, soybean, Brassica, and peanut, dicotyledons such as asparagus, barley, maize, oat, rice, sugarcane, tall fescue, and wheat, hypocotyl tissue, callus tissue, nodal explants, shoot meristem, cell cultures, immature embryos, scutellar tissue, and immature inflorescence.

In addition to or in conjunction with the methods described herein, the invention can include the use of Agrobacterium tumor-inducing (Ti) plasmid genes, which can contain a transfer DNA region (T-DNA), for engineering a plant cell's DNA. Agrobacterium can be used in the invention to produce Ti plasmid genes, and Agrobacterium strains used in the invention can include Agrobacterium tumefaciens strain C58, nopaline strains, octopine strains such as LBA4404, and agropine strains such as EHA101, EHA105, and EHA 109.

The invention can also include the use of promoters such as nopaline synthase (NOS) promoter, octopine synthase (OCS) promoter, caulimovirus promoters such as cauliflower mosaic virus (CaMV) 19S and 35S promoters, enhanced CaMV 35S promoter (e35S), figwort mosaic virus (FMV) 35S promoter, and promoters from the ribulose bisphosphate carboxylase (Rubisco) family such as Rubisco small subunit and Rubisco activase promoters in engineered plant cells.

### Conditions Used in Methods Disclosed Herein.

The present invention can use a substrate to culture the cells during transfection. The cells can be applied to, for example, a culture dish coated with a substrate that can promote growth and enrichment of the cells. Cells that do not adhere to the substrate can be washed away with media. Once adhered, the cells can spread and begin dividing on the substrate.

The substrate can comprise, for example, 1, 2, 3, 4, or 5 layers. The distance between two substrates layers may range from about 0.1 to about 20 mm, about 1 to about 10 mm, or about 1 to about 5 mm and each layer can be about 0.1, about 1, about 2, about 3, about 4, about 5, about 6, about 7, about 8, about 9, about 10, about 12, about 15, about 17, or about 20 mm.

The cells can be plated on a material made of, for example, plastic, glass, gelatin, polyacrylamide, or any combination thereof. The dishes used to the plate the cells can be, for example, microscope slides, culture plates, culture dishes, Petri dishes, microscope coverslips, an enclosed environmental chamber, a sealed culture dish, or multi-well culture dishes.

The binding surface layer of the substrate can be the portion of the substrate that is in contact with the cells. In some instances, the binding surface layer is the only layer, adjacent to the base layer, or separated from the base layer by one or more middle layers.

The binding surface layer of the substrate can comprise, for example, cell monolayers, cell lysates, biological materials associated with the extracellular matrix (ECM), gelatin, or any combination thereof.

Biological materials associated with the ECM can include, for example, collagen type I, collagen type IV, laminin, fibronectin, elastin, reticulin, hygroscopic molecules, glycosaminoglycanse, roteoglycans, glycocalyx, bovine serum albumin, Poly-L-lysine, Poly-D-lysine, or Poly-L-ornithine. The gelatin can be from an animal source, for example, the gelatin can porcine or bovine.

The monolayer of cells used in the substrate can be, for example, mammalian cells, endothelial cells, vascular cells, venous cells, capillary cells, human umbilical vein endothelial cells (HUVEC), human lung microvascular endothelial cells (HLMVEC). The cell lines can be obtained from a primary source or from an immortalized cell line. The monolayer of cells can be irradiated by ultraviolet light or X-ray sources to cause senescence of cells. The monolayer can also contain a mixture of one or more different cell types. The different cell types may be co-cultured together. One non-limiting example of co-culture is a combination of primary human endothelial cells co-cultured with transgenic mouse embryonic fibroblasts mixed to form a monolayer.

The binding surface layer of the substrate can contain, for example, a mixture of intracellular components. One method that can be used to obtain a mixture of intracellular components is lysis of the cells and collection of the cytosolic components. The lysed cells can be primary or immortalized. The lysed cells can be from either mono- or co-cultures.

The binding surface layer of the substrate can contain biological materials associated with the extracellular matrix (ECM) or binding moieties. For example, gelatin can be mixed directly with cells, binding moieties, biological materials associated with the ECM, or any combination thereof, to make a binding surface layer for the substrate. For example, the binding surface layer can be comprised of a gelatin mixed with a collagen.

The substrate can have one or more middle layers. The middle layer of the substrate can be one or more monolayers of cells. The cells of the monolayer can be of varying origin. For example, the middle layer of the substrate can be made by growing a confluent monolayer of mouse embryonic fibroblasts on the base layer and then growing another layer of cells, for example, the binding surface layer, on top of the confluent mouse embryonic fibroblasts.

A feeder layer can be used in the substrate for growth or reprogramming of the cells. A feeder layer can sit adjacent to a base layer and can be separated from the binding surface layer of the substrate. The feeder layer can be a monolayer of feeder cells. The cells of the monolayer can be of varying origin. For example, the feeder layer can be made by growing a monolayer of human endothelial cells or mouse embryonic fibroblasts on a base layer.

Conjugation of layers of the substrate can be done by allowing cells to grow in a monolayer on top of the base layer or middle layer. Conjugation of layers can also be done by pre-treating the surface with a surface of either net positive, net negative, or net neutral charge. The conjugation procedure can be aided by chemical moieties, linkers, protein fragments, nucleotide fragments, or any combination thereof.

The media used for growing the cells can be supplemented or made with culture media that has been collected from cell cultures, blood plasma, or any combination thereof. The enrichment media can be, for example, Plating Culture Medium, Type R Long Term Growth Medium, Type DF Long Term Growth Medium, Type D Long Term Growth Medium, and MEF—Enrichment Medium, or any combination thereof. The enrichment medium can contain, for example, a primary nutrient source, animal serum, ions, elements, calcium, glutamate, magnesium, zinc, iron, potassium, sodium, amino acids, vitamins, glucose, growth factors, hormones, tissue extracts, proteins, small molecules, or any combination thereof. In some embodiments, the culture media used for transfection does not contain serum.

Non-limiting examples of amino acids include essential amino acids, phenylalanine, valine, threonine, tryptophan, isoleucine, methionine, leucine, lysine, and histidine, arginine, cysteine, glycine, glutamine, proline, serine, tyrosine, alanine, asparagine, aspartic acid, glutamic acid, or any combination thereof.

Non-limiting examples of growth factors include Epidermal Growth Factor (EGF), Nerve Growth Factor (NGF), Brain Derived Neurotrophic Factor (BDNF), Fibroblast Growth Factor (FGF), Stem Cell Factor (SCF), Insulin-like Growth Factor (IGF), Transforming Growth Factor-beta (TGF-β), or any combination thereof.

Non-limiting examples of hormones include peptide hormones, insulin, steroidal hormones, hydrocortisone, progesterone, testosterone, estrogen, dihydrotestosterone, or any combination thereof.

Non-limiting examples of tissue extracts include pituitary extract. Non-limiting examples of small molecule additives include sodium pyruvate, endothelin-1, transferrin, cholesterol, or any combination thereof.

The culturing conditions in a method of the invention can be adjusted to simulate oxygen and pressure levels found, for example, in pathological conditions. The oxygen level used during culturing conditions can be, for example, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% oxygen in the incubator. In some embodiments, the cells can be grown under hypoxic conditions during transfection.

The culturing condition in a method of the invention can be adjusted to simulate the pressure found, for example, in pathological conditions. The pressure used during culturing conditions can be about 0 PSI, about 0.1 PSI, about 0.15 PSI about 0.2 PSI, about 0.25 PSI, about 0.3 PSI, about 0.35 PSI, about 0.4 PSI, about 0.45 PSI, 0.5 PSI, about 0.55 PSI, about 0.6 PSI, about 0.65 PSI, about 0.7 PSI, about 0.75 PSI, about 0.8 PSI, about 0.85 PSI, about 0.9 PSI, about 0.95 PSI, about 1 PSI, about 1.1 PSI, about 1.2 PSI, about 1.3 PSI, about 1.4 PSI, about 1.5 PSI, about 1.6 PSI, about 1.7 PSI, about 1.8 PSIG, about 1.9 PSI, about 2 PSI, about 2.1 PSI, about 2.2 PSI, about 2.3 PSI, about 2.4 PSI, about 2.5 PSI, about 2.6 PSI, about 2.7 PSI, about 2.8 PSI, about 2.9 PSI, about 3 PSI, about 3.5 PSI, about 4 PSI, about 4.5 PSI, about 5 PSI, about 6 PSI, about 7 PSI, about 8 PSI, about 9 PSI, or about 10 PSI. A pressure used in a method disclosed herein can be an above atmospheric pressure value. A pressure used in a method disclosed herein can be positive pressure.

The culturing condition in a method of the invention can be adjusted to simulate the pressure found, for example, in pathological conditions. The pressure used during culturing conditions can be a PSI gauge (PSIG) reading of, for example, about 0.5 PSIG, about 0.6 PSIG, about 0.7 PSIG, about 0.8 PSIG, about 0.9 PSIG, about 1 PSIG, about 1.1 PSIG, about 1.2 PSIG, about 1.3 PSIG, about 1.4 PSIG, about 1.5 PSIG, about 1.6 PSIG, about 1.7 PSIG, about 1.8 PSIG, about 1.9 PSIG, about 2 PSIG, about 2.5 PSIG, about 3 PSIG, about 3.5 PSIG, about 4 PSIG, about 4.5 PSIG, about 5 PSIG, about 6 PSIG, about 7 PSIG, about 8 PSIG, about 9 PSIG, about 10 PSIG, about 15 PSIG, about 20 PSIG, about 25 PSIG, about 30 PSIG, about 35 PSIG, about 40 PSIG, about 45 PSIG, about 50 PSIG, or about 55 PSIG.

The pressure used during culturing conditions can be, for example, about 3.45 kPa, about 4.14 kPa, about 4.83 kPa, about 5.52 kPa, about 6.21 kPa, about 6.89 kPa, about 7.58 kPa, about 8.27 kPa, about 8.96 kPa, about 9.65 kPa, about 10.3 kPa, about 11 kPa, about 11.7 kPa, about 12.4 kPa, about 13.1 kPa, about 13.8 kPa, about 17.2 kPa, about 20.7 kPa, about 24.1 kPa, about 27.6 kPa, about 31 kPa, about 34.4 kPa, about 41.4 kPa, about 48.3 kPa, about 55.2 kPa, about 62.1 kPa, about 68.9 kPa, about 103 kPa, about 138 kPa, about 172 kPa, about 207 kPa, about 241 kPa, about 276 kPa, about 310 kPa, about 345 kPa, or about 379 kPa.

The pressure used in a method of the invention can be delivered continuously or via pulses of pressure produced by repeated depressurizations and pressurizations of an incubator used in the method. The pulses of pressure can be separated by, for example, about 1 minute, about 1.5 minutes, about 2 minutes, about 2.5 minutes, about 3 minutes, about 3.5 minutes, about 4 minutes, about 4.5 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 32 minutes, about 34 minutes, about 36 minutes, about 38 minutes, about 40 minutes, about 45 minutes, about 50 minutes, about 55 minutes, about 1 hour, about 1.5 hours, about 2 hours, about 2.5 hours, about 3 hours, about 3.5 hours, about 4 hours, about 4.5 hours, or about 5 hours.

The pH of the media used in a method of the invention can be, for example, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.55, about 4.6, about 4.7, about 4.8, about 4.9, about 5, about 5.5, about 6, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.5, about 9, about 9.5, about 10, about 10.5, or about 11 pH units.

The viscosity of the media can be adjusted by, for example, at least 0.001 Pascal-second (Pa·s), at least 0.001 Pa·s, at least 0.0009 Pa·s, at least 0.0008 Pa·s, at least 0.0007 Pa·s, at least 0.0006 Pa·s, at least 0.0005 Pa·s, at least 0.0004 Pa·s, at least 0.0003 Pa·s, at least 0.0002 Pa·s, at least 0.0001 Pa·s, at least 0.00005 Pa·s, or at least 0.00001 Pa·s depending on the cell types being cultured.

The oxygen solubility of the media can be, for example, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%.

In some embodiments, a culture media used for a method described herein can contain, for example, an L-alanine-L-glutamine dipeptide, B27 TM supplement, human bFGF, human EGF, human HGH, 1 mg/mL human insulin, 0.55 mg/mL human transferrin, 0.5 μg/mL sodium selenite, beta-mercaptoethanol, non-essential amina acids solution, and high glucose media.

In some embodiments, for PBMC or CD8+ cell culture, a culture media for a method described herein can contain, for example, PHA-P (10 μg/mL), IL-2 (100 U/mL), IL-4 (20 ng/mL), IL-15 (100 ng/mL), GM-CSF (20 ng/mL), and LPS (100 ng/mL).

The oxygen concentration used in a method disclosed herein can be used to mimic oxygen concentration found in, for example, solid tumors (about 1.1%), muscle (about 3.8%), prostate (about 3.9%), brain (about 4.4%), peripheral tissues (about 5.3%), venous blood (about 5.3%), lung (about 5.6%), bone marrow (about 6.4%), intestinal tissue (about 7.6%), kidney (about 9.5%), and arterial blood (about 13.2%).

The pressure conditions used in a method disclosed herein can be used to mimic the interstitial fluid pressure found in, for example, normal breast (about 0.02 PSI), normal skin (about 0.04 PSI), lymphoma (about 0.14 PSI), brain tumors (about 0.15 PSI), sarcoma (about 0.17 PSI), lung carcinoma (about 0.25 PSI), rectal carcinoma (about 0.33 PSI), breast carcinoma (about 0.37 PSI), head and neck carcinoma (about 0.41 PSI), metastatic melanoma (about 0.43 PSI), colorectal carcinoma liver metastases (about 0.43 PSI), cervical carcinoma (about 0.44 PSI), ovarian carcinoma (about 0.48 PSI), and renal cell carcinoma (about 0.72 PSI).

### Therapeutic Uses.

Subjects can be, for example, elderly adults, adults, adolescents, pre-adolescents, children, toddlers, infants. Subjects can be non-human animals, for example, a subject can be a mouse, rat, cow, horse, donkey, pig, sheep, dog, cat, or goat. A subject can be a patient.

A method disclosed herein can be used to identify a therapeutic, a biomarker, a genetic mutation, or a therapeutic target for, for example, stem cell differentiation or differentiation of various cell types.

Genomic, proteomic, and metabolic analysis can be conducted on the transfected cells to, for example, identify biomarkers that can be used for development of cancer therapies, drug development, cancer vaccines, cancer screening, diagnostics, personalized antibody development, hematopoietic stem cell transplantation, organ transplantation, or cardiovascular disease treatment. A method described herein can be used to induce phenotypic and genotypic changes in cells to determine the effect of cancer therapies. The cancer therapies can include, for example, chemotherapeutics or gene therapy.

Non-limiting examples of cancers that can be analyzed in a method disclosed herein include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyo sarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

Methods that can be used to determine the presence of, for example, biological markers or transfection of desired genes can include, for example, qPCR, RT-PCR, immunofluorescence, immunohistochemistry, western blotting, high-throughput sequencing, or mRNA sequencing.

### Computer Systems.

A method of the invention can be used to, for example, sequence, image, or characterize the transfected cells. Further methods can be found in PCT/US14/13048, the entirety of which is incorporated herein by reference.

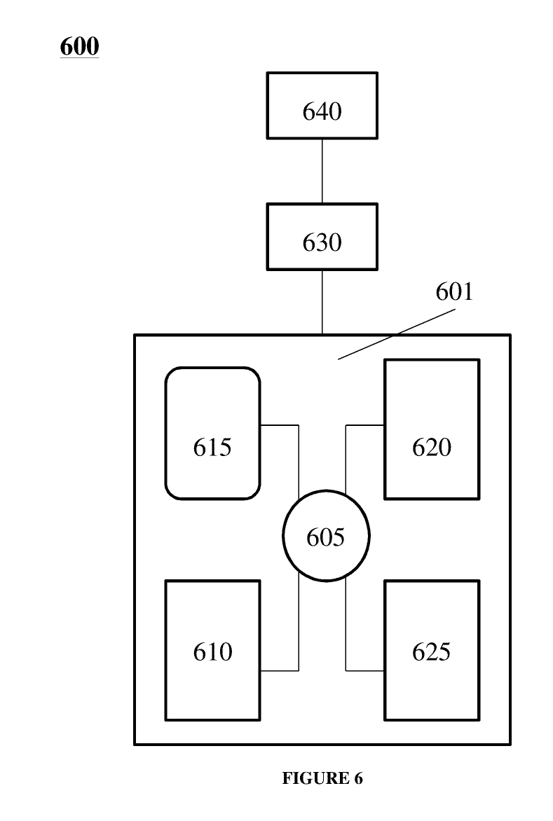

The invention provides a computer system that is configured to implement the methods of the disclosure. The system can include a computer server (“server”) that is programmed to implement the methods described herein. FIG. 6 depicts a system 600 adapted to enable a user to detect, analyze, and process images of cells and sequence cells. The system 600 includes a central computer server 601 that is programmed to implement exemplary methods described herein. The server 601 includes a central processing unit (CPU, also “processor”) 605 which can be a single core processor, a multi core processor, or plurality of processors for parallel processing. The server 601 also includes memory 610 (e.g. random access memory, read-only memory, flash memory); electronic storage unit 615 (e.g. hard disk); communications interface 620 (e.g. network adaptor) for communicating with one or more other systems; and peripheral devices 625 which may include cache, other memory, data storage, and/or electronic display adaptors. The memory 610, storage unit 615, interface 620, and peripheral devices 625 are in communication with the processor 605 through a communications bus (solid lines), such as a motherboard. The storage unit 615 can be a data storage unit for storing data. The server 601 is operatively coupled to a computer network (“network”) 630 with the aid of the communications interface 620. The network 630 can be the Internet, an intranet and/or an extranet, an intranet and/or extranet that is in communication with the Internet, a telecommunication or data network. The network 630 in some cases, with the aid of the server 601, can implement a peer-to-peer network, which may enable devices coupled to the server 601 to behave as a client or a server. The microscope and micromanipulator can be peripheral devices 625 or remote computer systems 640.

The storage unit 615 can store files, such as individual images, time lapse images, data about individual cells, cell colonies, or any aspect of data associated with the invention. The data storage unit 615 may be coupled with data relating to locations of cells in a virtual grid.

The server can communicate with one or more remote computer systems through the network 630. The one or more remote computer systems may be, for example, personal computers, laptops, tablets, telephones, Smart phones, or personal digital assistants.

In some situations the system 600 includes a single server 601. In other situations, the system includes multiple servers in communication with one another through an intranet, extranet and/or the Internet.

The server 601 can be adapted to store cell profile information, such as, for example, cell size, morphology, shape, migratory ability, proliferative capacity, kinetic properties, and/or other information of potential relevance. Such information can be stored on the storage unit 615 or the server 601 and such data can be transmitted through a network.

Methods as described herein can be implemented by way of machine (e.g., computer processor) computer readable medium (or software) stored on an electronic storage location of the server 601, such as, for example, on the memory 610, or electronic storage unit 615. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610. Alternatively, the code can be executed on a second computer system 640.

Aspects of the systems and methods provided herein, such as the server 601, can be embodied in programming. Various aspects of the technology may be thought of as “products” or “articles of manufacture” typically in the form of machine (or processor) executable code and/or associated data that is carried on or embodied in a type of machine readable medium (e.g., computer readable medium). Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

### EXAMPLES

### Example 1: Transfection Efficiency Using a Method Disclosed Herein

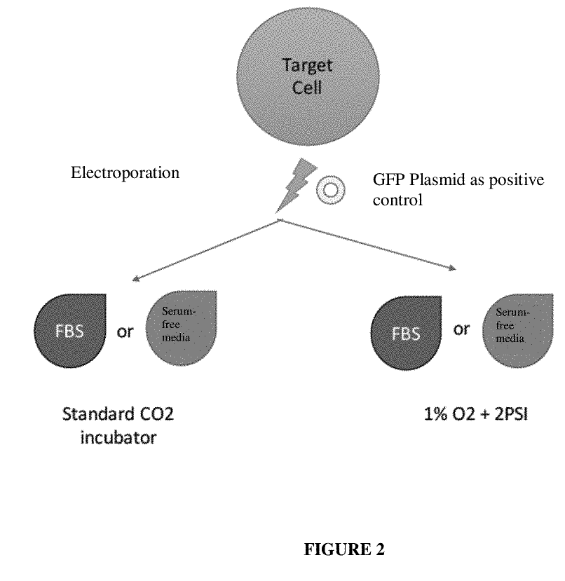

FIG. 2 depicts an experimental procedure for comparison of electroporation versus a method of the invention, which includes hypoxic and high pressure conditions. The cells were cultured in either a standard CO₂ incubator or under hypoxic and positive pressure conditions. The cells were further cultured in media and a substrate that contained FBS or serum-free media and a serum-free substrate.

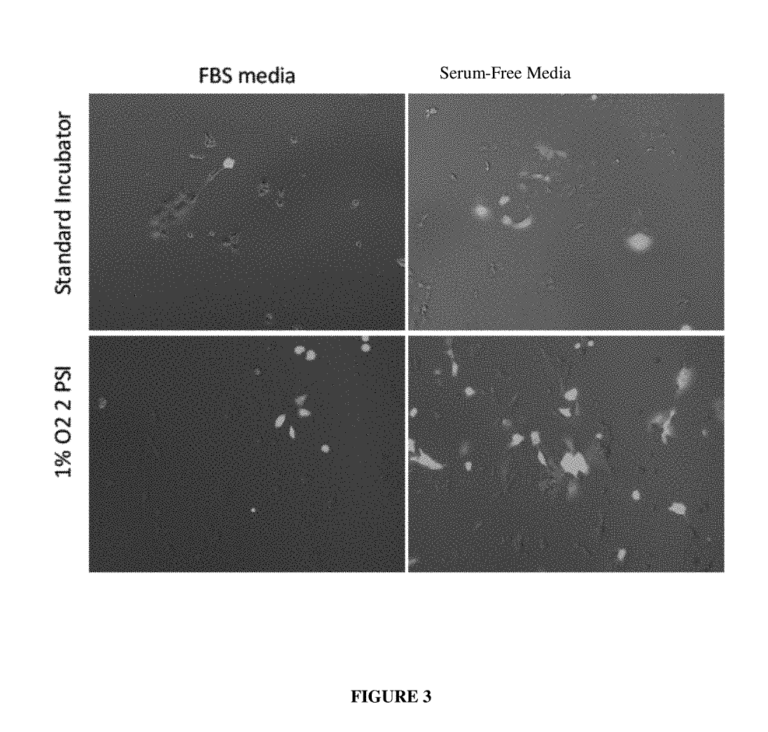

FIG. 3 depicts the results of the transfection of DU145 (human prostate cancer) cells. 5×10̂6 cells/mL were transfected with 0.5 μg GFP at 1260 V for 40 ms with 2 pulses. After transfection, the cells were evenly split across the tested conditions and cultured for 48 hours prior to imaging and assessment of transfection efficiency. FIG. 3 shows that transfection of the cells under hypoxic and high pressure conditions, along with culturing in serum-free media allowed for the greatest transfection efficiency as indicated by the brightest GFP staining in the bottom-right panel.

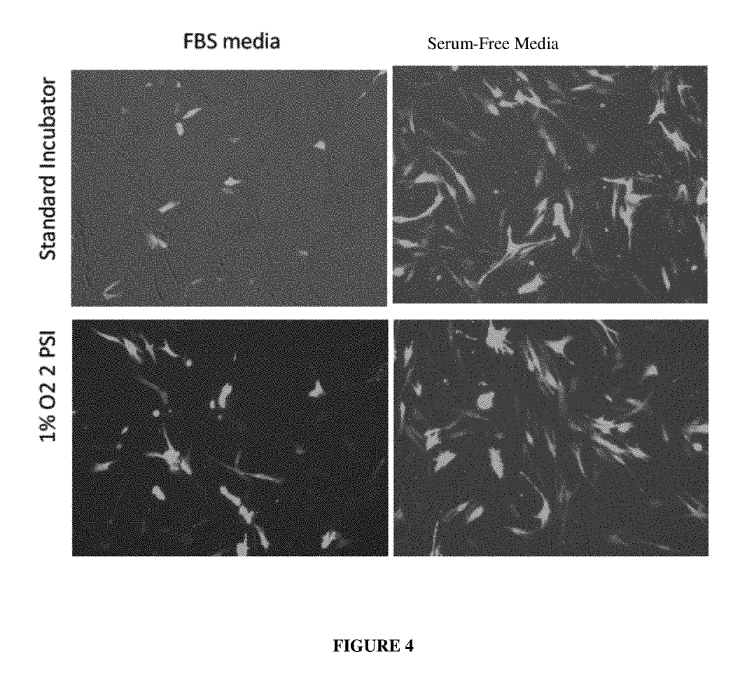

FIG. 4 depicts the results of the transfection of human dermal fibroblast cells. 5×10̂6 cells/mL were transfected with 0.5 μg GFP at 1000 V for 30 ms with 1 pulse. After transfection, the cells were evenly split across the tested conditions and cultured for 48 hours prior to imaging and assessment of transfection efficiency. FIG. 4 shows that transfection of the cells under hypoxic and high pressure conditions, along with culturing in serum-free media allowed for the greatest transfection efficiency as indicated by the brightest GFP staining in the bottom-right panel.

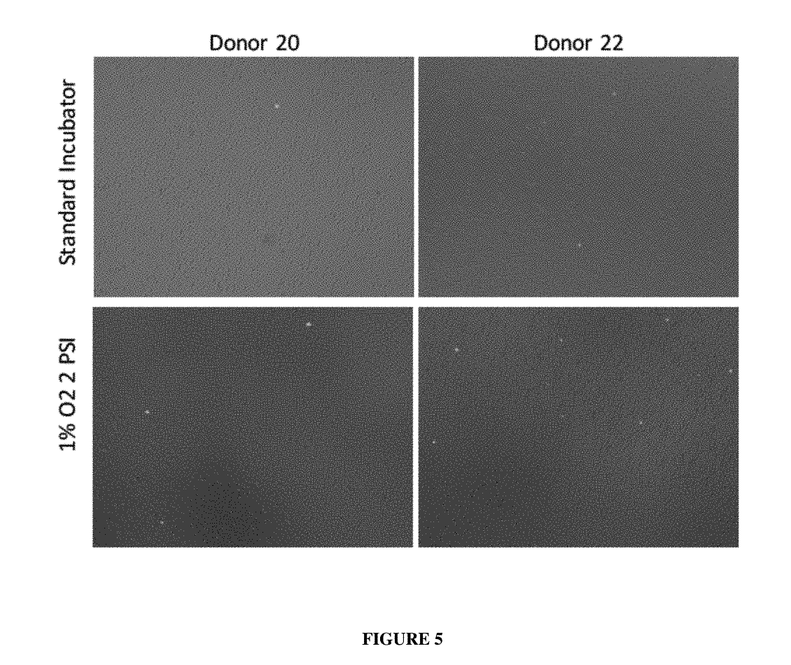

FIG. 5 depicts the results of the transfection of healthy donor peripheral blood mononuclear cells (PBMCs). 2×10̂7 cells/mL were transfected with 1 μg GFP at 1500 V for 10 ms with 1 pulse. After transfection, the cells were evenly split across the tested conditions and cultured for 48 hours prior to imaging and assessment of transfection efficiency. FIG. 5 shows that transfection of the cells under hypoxic and high pressure conditions, along with culturing in serum-free media allowed for the greatest transfection efficiency as indicated by the brightest GFP staining in the bottom panels.

TABLE 1 below provides a quantitative analysis of the change in fold expression of the GFP plasmid using different methods.

TABLE 1
Fold Expression change of GFP

| Transfection Condition | DU145 | PC3 | Panc10 | Dermal fibroblasts |
|---|---|---|---|---|
| Standard, FBS | 1 | 1 | 1 | 1 |
| Standard, Serum-free | 5.56 | 1.88 | 0.574 | 3.81 |
| media and substrate | | | | |
| 1% O₂ 2 PSI, FBS | 2.55 | 5.033 | 15.731 | 5.018 |
| 1% O₂ 2 PSI, Serum-free | 14.2 | 4.234 | 36.533 | 6.642 |
| media and substrate | | | | |

### Example 2: Transfection of Human Dermal Fibroblasts

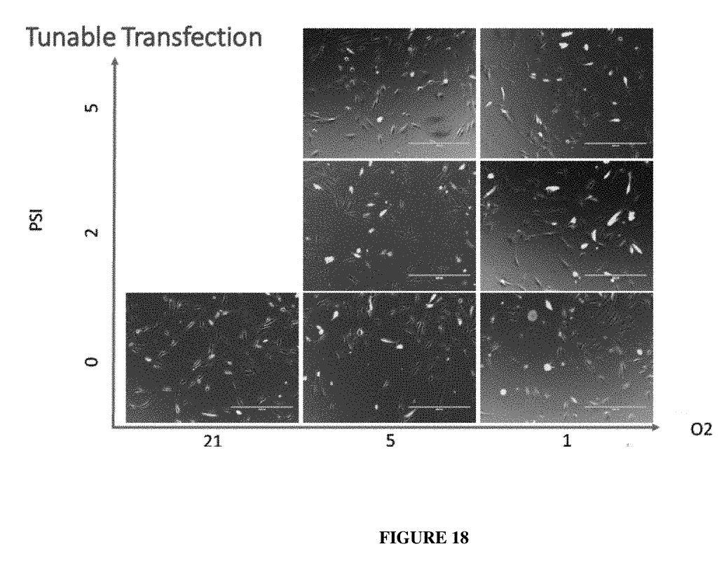

FIG. 18 depicts the transfection of human dermal fibroblasts under 21% O₂ and 0 PSI, 5% O₂ and 0, 2, or 5 PSI, and 1% O₂, and 0, 2, or 5 PSI. The cells were transfected with a GFP plasmid and imaged 48 hours post-transfection. The experiments were repeated in triplicate. FIG. 18 shows that transfection efficiency increased at lower oxygen and higher pressure conditions as indicated by brighter GFP expression.

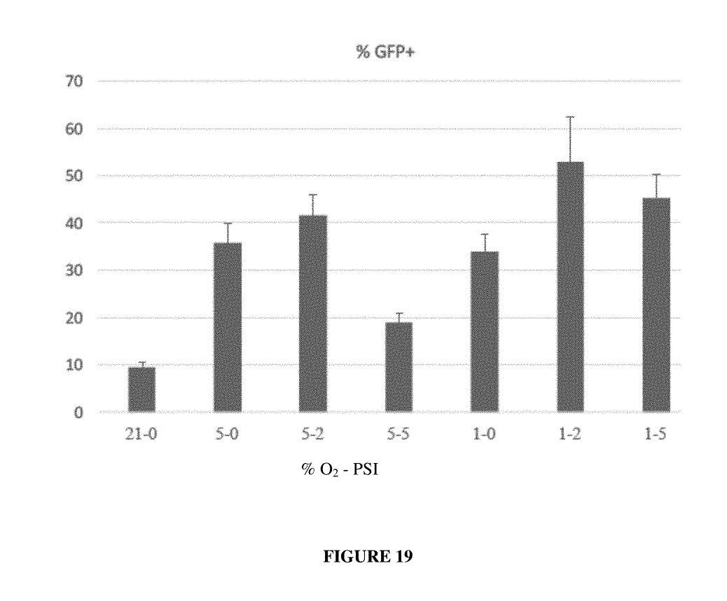

FIG. 19 provides a quantitative analysis of GFP transfection of human dermal fibroblasts grown under various hypoxic and high pressure conditions in FIG. 18. The results indicate that cells grown under the hypoxic and positive pressure conditions provided higher GFP transfection efficiency than cells grown under 21% O₂ and 0 PSI.

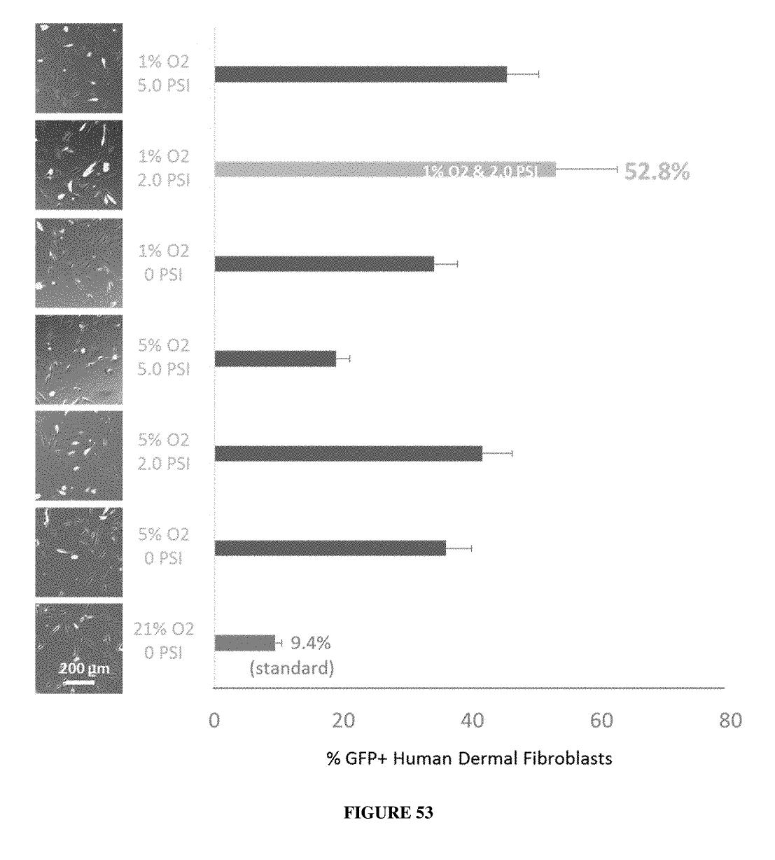

FIG. 53 shows the transfection of human dermal fibroblasts using electroporation of a GFP plasmid. The cells were cultured under several conditions of low oxygen and high pressure. The results indicated that 1% oxygen and 2 PSI pressure provided the greatest proportion of transfected cells (52.8% GFP+ cells versus 9.4% GFP+ under standard culture conditions).

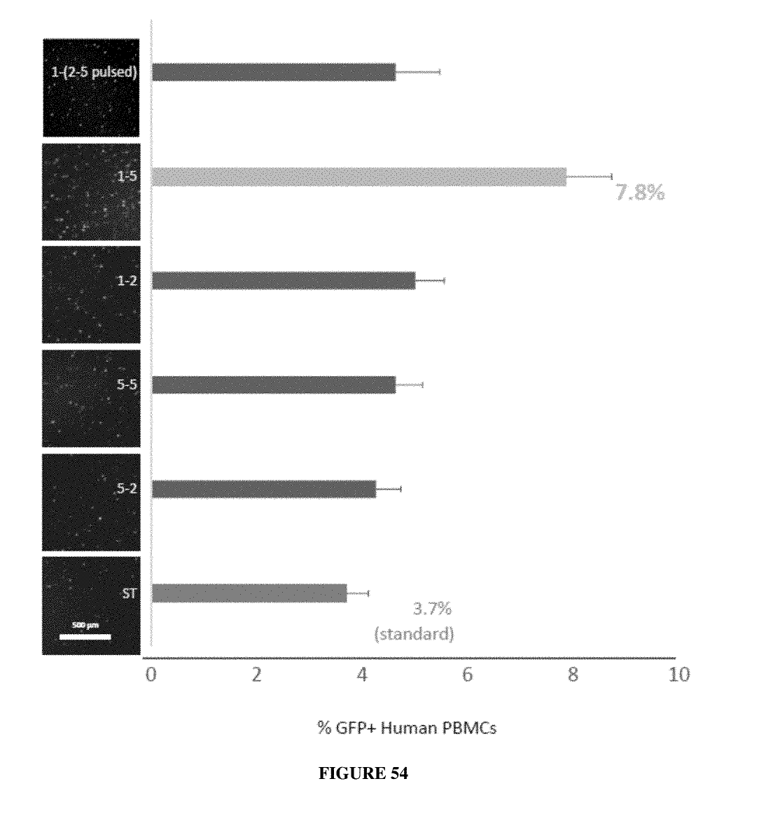

FIG. 54 shows the transfection of PBMCs using electroporation of a GFP plasmid. The cells were cultured under several conditions of low oxygen and high pressure. The results indicated that 1% oxygen and 5 PSI pressure provided the greatest proportion of transfected cells (7.8% GFP+ cells versus 3.7% GFP+ under standard culture conditions).

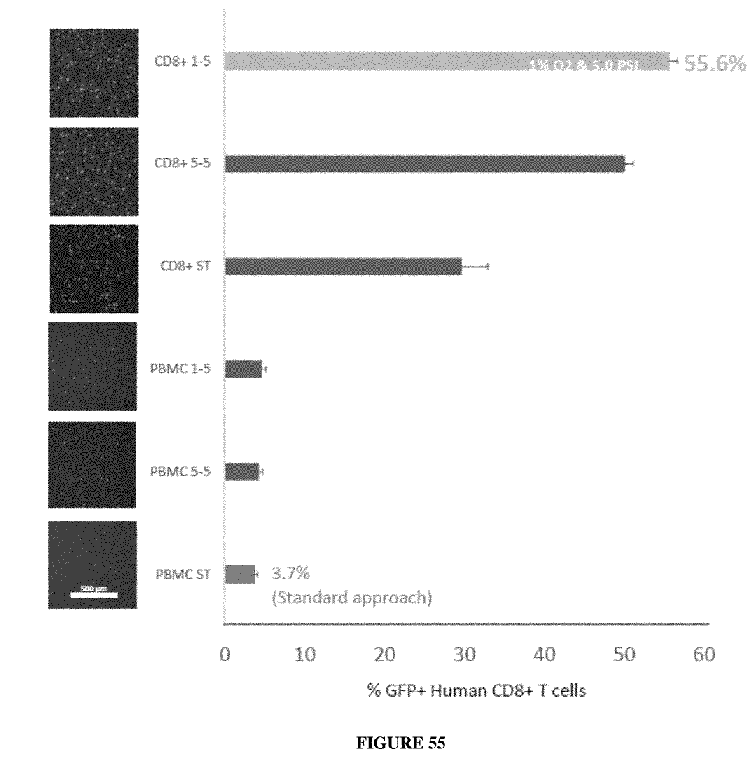

FIG. 55 shows the transfection of activated CD8+ T-cells using electroporation of a GFP plasmid. The cells were cultured under several conditions of low oxygen and high pressure. The results indicated that 1% oxygen and 5 PSI pressure provided the greatest proportion of transfected cells (55.6% GFP+ cells versus 3.7% GFP+ under standard culture conditions).

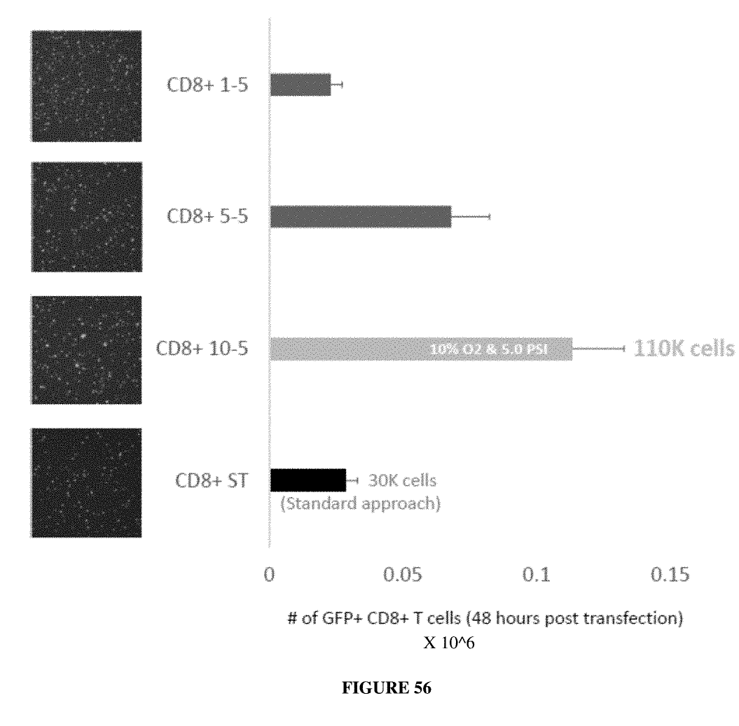

FIG. 56 shows the post-transfection effects of CD8+ T-cells using low oxygen and high pressure conditions. The results indicated that lower oxygen (10% O₂) and positive pressure (5 PSI) promotes cell proliferation post-transfection (1.1×10⁵ cells under higher oxygen and positive pressure versus 3×10⁴ cells under standard conditions 2 days after transfection).

### Example 3: Transfection of Immune Cells

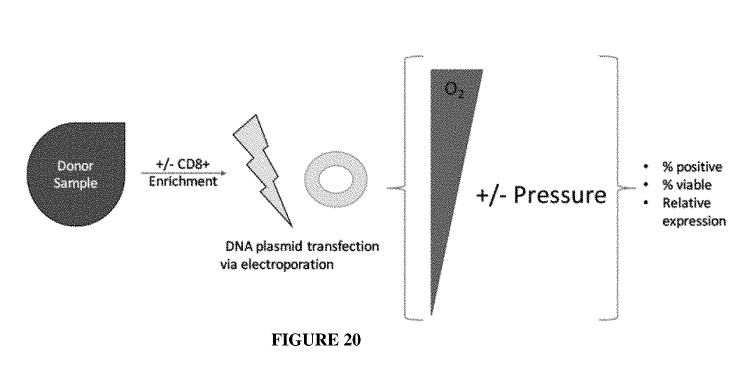

FIG. 20 depicts a sample workflow for transfection of immune cells using a method disclosed herein. FIG. 20 shows that after a sample is obtained from a donor, the sample can be enriched for CD8+ cells. A DNA plasmid is transfected using electroporation. The transfected cells are then subjected to decreasing oxygen levels and either 0 PSI or high pressure conditions. Then, the percent GFP-positive cells, percent viable cells, and relative expression of GFP in the cells are assessed.

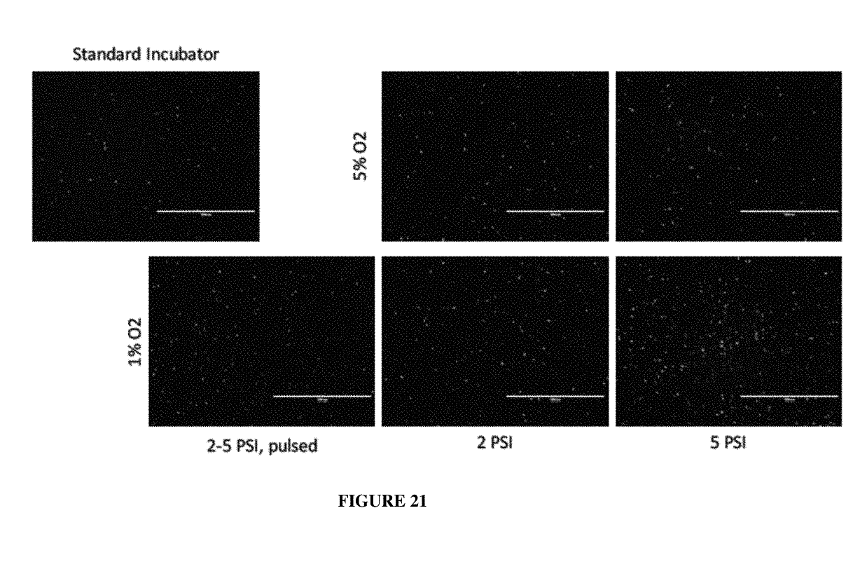

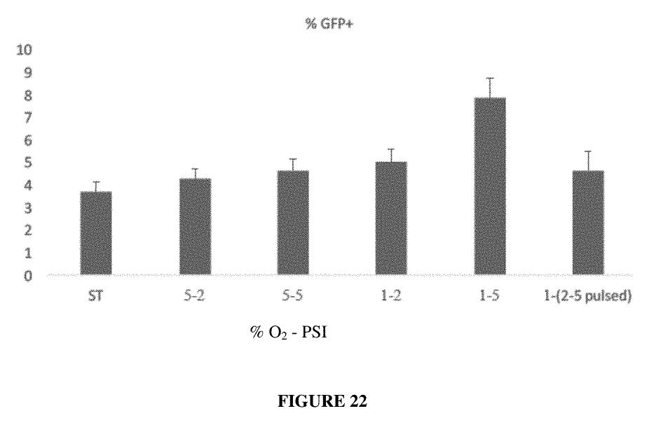

FIG. 21 shows the transfection of peripheral blood mononuclear cells (PBMC) with a GFP plasmid using electroporation of cells at passage zero. The 2-5 PSI pulsed pressure condition was performed at a frequency of 30 minute pulses. The experiments were repeated four times. FIG. 22 depicts a quantification of the results from FIG. 21 indicating that there was an almost 2.5-fold increase in GFP-positive cells using the hypoxic and positive pressure conditions compared to standard incubator conditions.

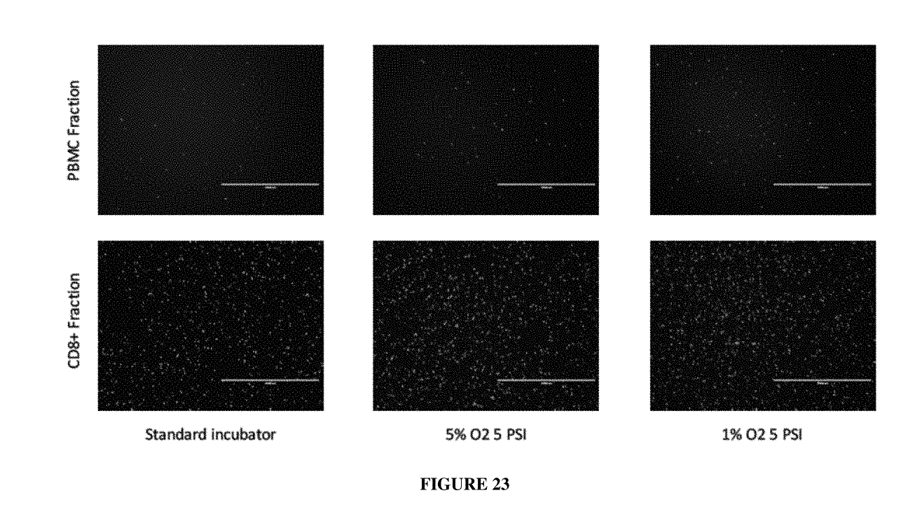

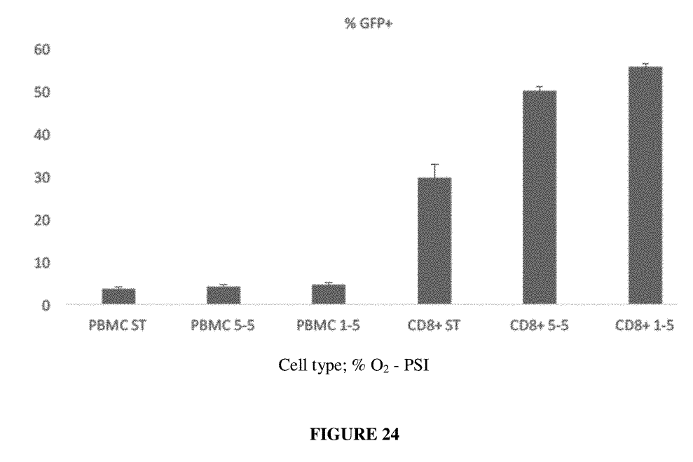

FIG. 23 shows a comparison between the transfection of CD8+ cells enriched from PBMC and PBMC with a GFP plasmid using electroporation of cells at passage zero. The experiment was performed in triplicate. FIG. 24 depicts a quantification of the results from FIG. 23 indicating that enriched CD8+ cells have higher transfection efficiency than the PBMC. “ST” in FIG. 24 denotes standard culture conditions.

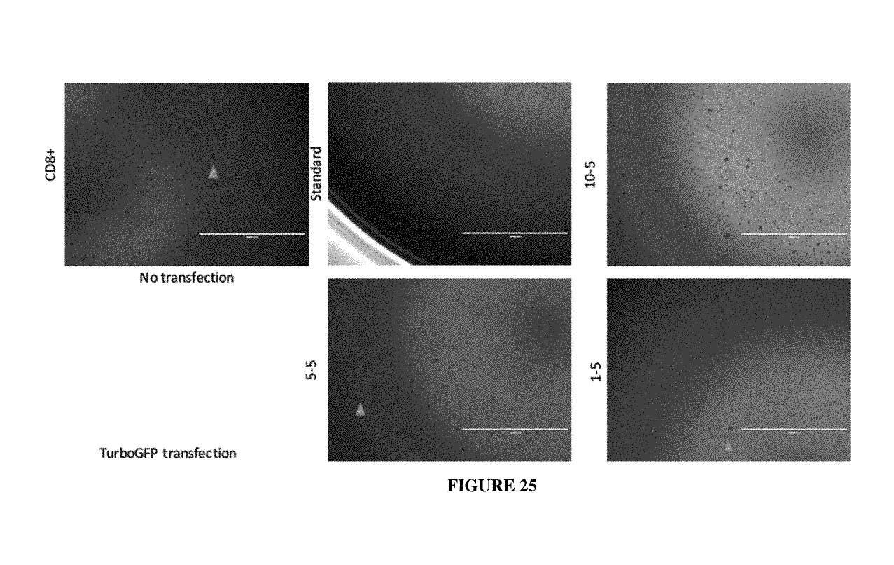

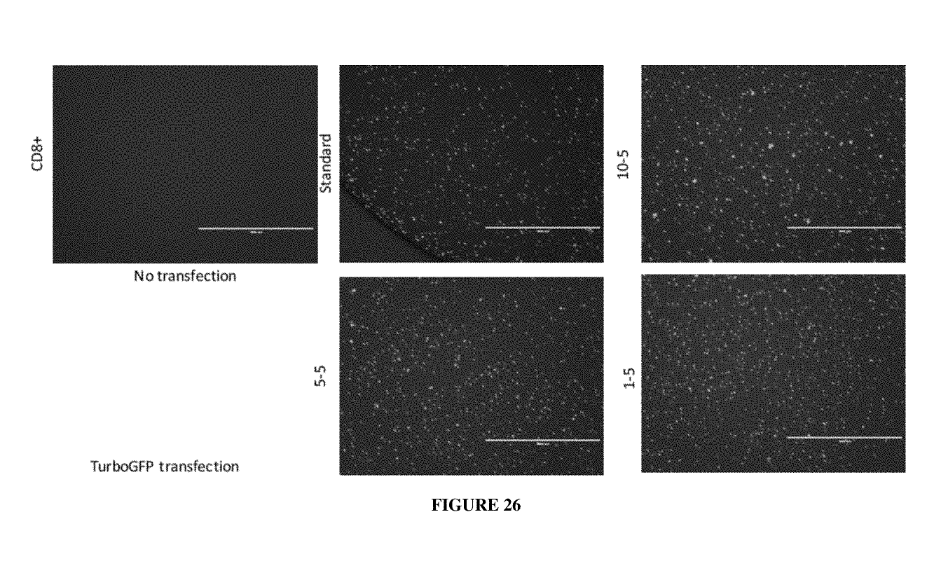

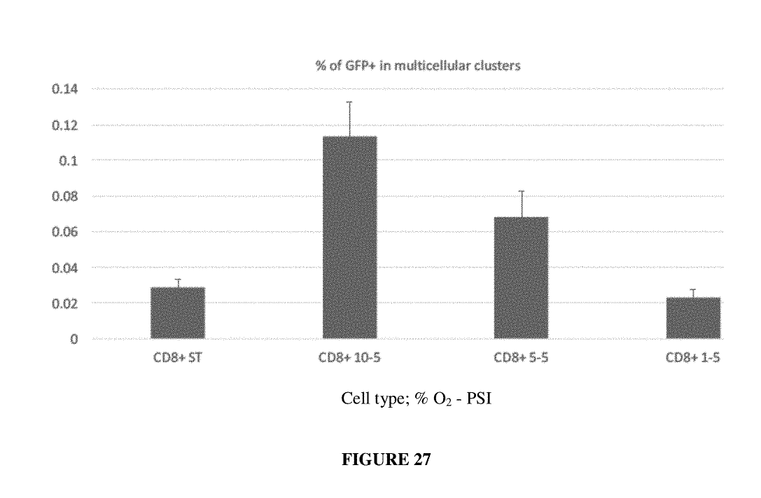

FIG. 25 shows that the GFP-transfected CD8+ cells cultured under hypoxic and positive pressure conditions developed more multicellular clusters than did cells grown at standard incubator conditions. In each panel, the first number indicates the oxygen level; the second number represents the pressure level in PSI. The top left panel is a control without transfection. FIG. 26 shows the percent GFP in the multicellular clusters in cells grown under hypoxic and positive pressure conditions compared to cells grown under standard incubator conditions. FIG. 27 is a quantification of the results of FIG. 26. “ST” in FIG. 27 denotes standard culture conditions.

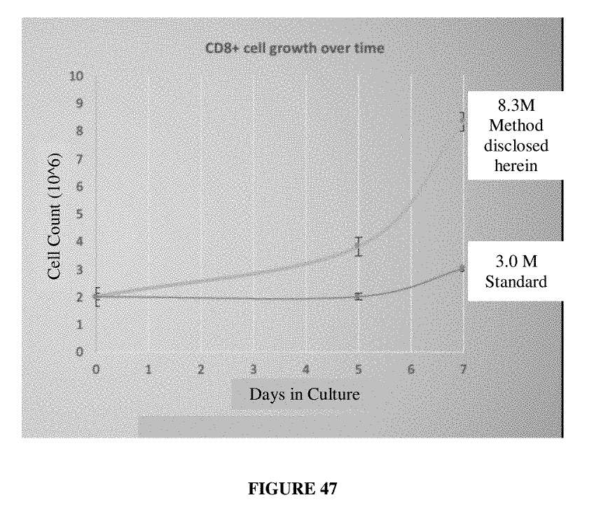

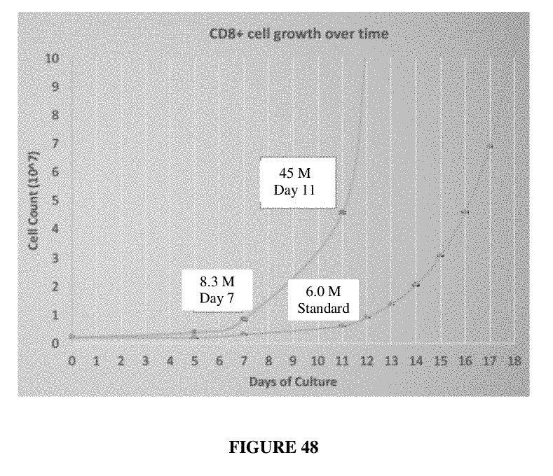

FIG. 47 shows that hypoxic and positive pressure conditions can lead to greater enrichment of CD8+ cells from fresh blood samples than culture under standard incubator conditions. For example, FIG. 47 shows that 8.3 million CD8+ cells were obtained after one week under hypoxic and positive pressure conditions versus 3 million CD8+ cells obtained under standard conditions. An expanded culture time up to 11 days is shown in FIG. 48 indicating that the culture under hypoxic and positive pressure conditions generates more CD8+ cells from whole blood than culture under standard conditions.

### Example 4: Immune Cell Genome Editing

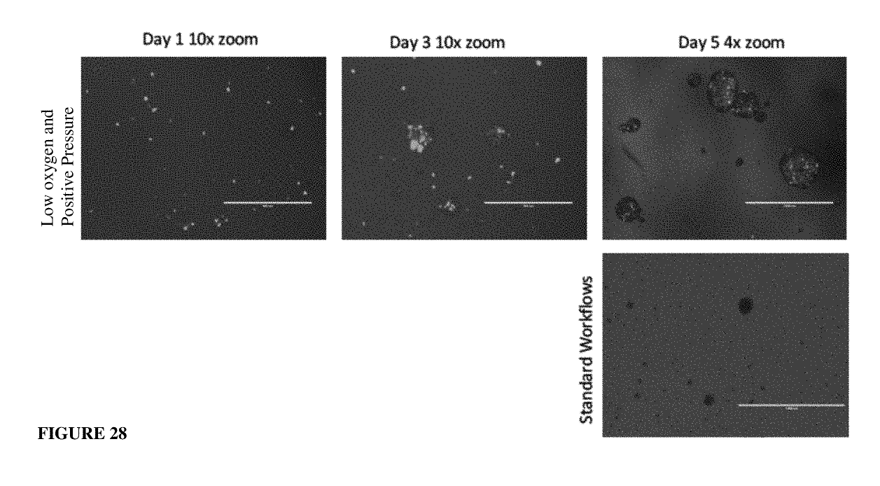

A method disclosed herein can be used to introduce the CRISPR/Cas9 system into immune cells, for example, CD8+ T-cells, as shown in FIGS. 28-30. PBMCs were freshly isolated from healthy donors, and post-cell counting PBMCs were enriched for CD8+ cells. The CD8+ cells were cultured at 2 million cells/mL in IL-2 containing media in a standard incubator, or with PBMC media under low oxygen and positive pressure conditions. Once the cells demonstrated doubling times of 36-48 hours, the cells were transfected at 20 million cells/ml with 1 μg/μl CTLA4 CRISPR Knockout and a GFP HDR DNA plasmid. The cells were expanded under low oxygen and positive pressure conditions or under conventional culture conditions. Images were taken 1, 3, and 5 days post-transfection prior to cell splitting and counting. Quantification was performed through cell counting across entire wells and assessed as GFP+ multicellular clusters/total multicellular clusters at 5 days post-transfection as well as total cell count at 5 days post-transfection.

FIG. 28 shows that when a CRISPR/Cas9 system was used to knockout CTLA4, and knock-in GFP using homology-directed repair, the transfection efficiency of the CRISPR/Cas9 system was higher in the cells grown under hypoxic and high pressure conditions than in standard incubator conditions. This is indicated by the bright GFP signal seen in the top panels of FIG. 28. The results also indicate that the GFP expression persisted through subsequent expansion of the CD8+ cells for at least five days. The experiment was repeated four times, and the cells were transfected at passage 1+ while in the exponential growth phase.

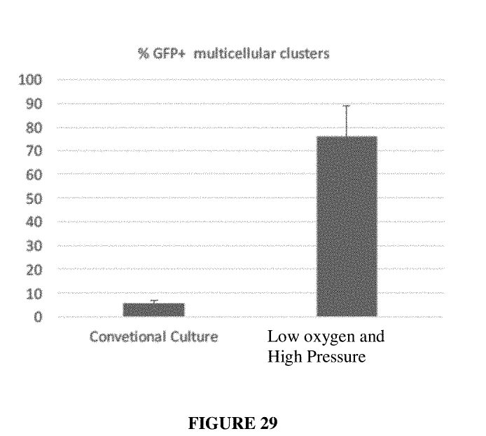

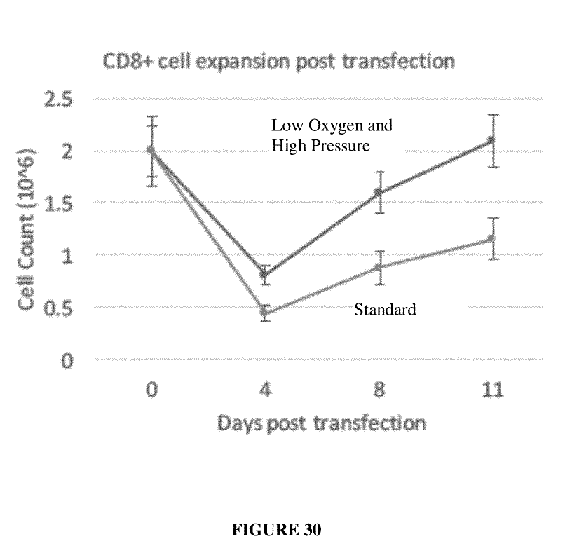

FIGS. 29 and 30 provide a quantification of the results of from FIG. 28. FIG. 29 shows that the cells grown under hypoxic and positive pressure conditions developed a higher percentage of GFP-positive multicellular clusters than the cells grown at standard culture conditions. FIG. 30 shows that the proliferation of the CD8+ cells grown under hypoxic and positive pressure conditions was enriched over the cells grown under standard incubator conditions. The error bars represent standard deviation.

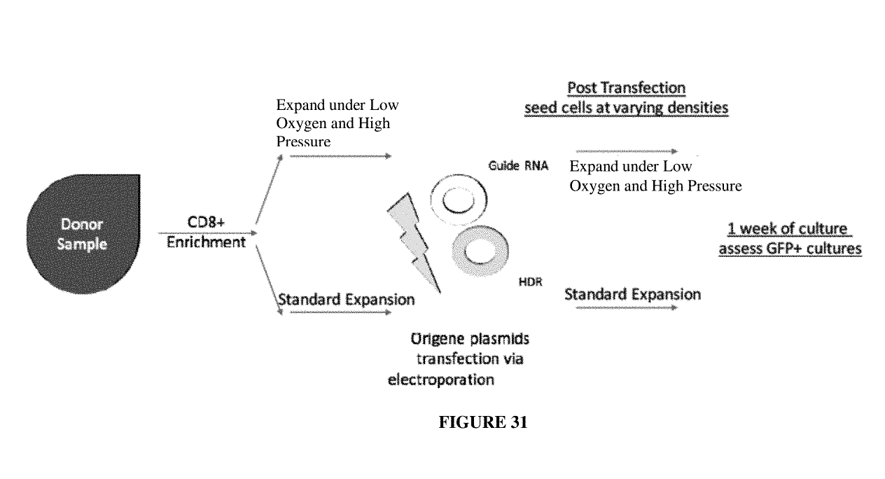

FIG. 31 depicts a limited dilution assay workflow to assess GFP-positive colonies using the CRISPR/Cas9 system. A sample is taken from a donor and then subjected to enrichment for CD8+ cells. Then, the cells are split to be cultured under either positive pressure and low oxygen conditions or under standard incubator conditions. The cells are then transfected using electroporation using a guide RNA and a homology-directed repair plasmid to effect the genomic GFP insertion. The cells are then expanded under either positive pressure and low oxygen conditions or under standard incubator conditions. After one week, the GFP-positive cells are assessed.

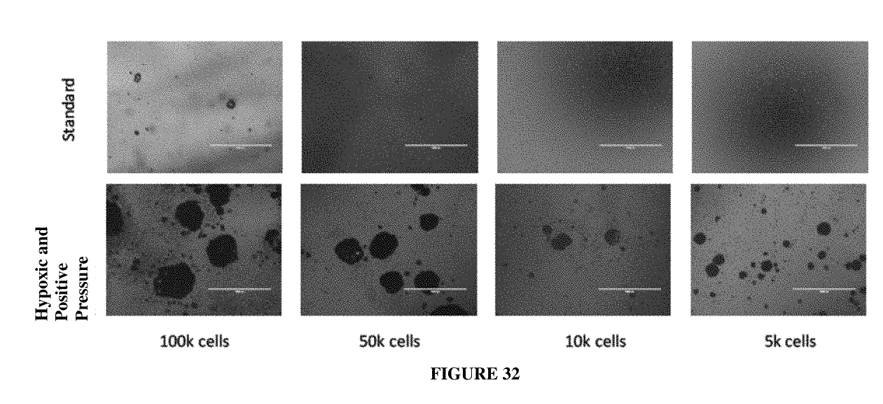

FIG. 32 shows that less input of T-cells still allowed for successful genome editing of the CD8-positive T-cells as indicated by the GFP signal visible in the cells even at the lowest concentration of 5000 cells. TABLE 2 below provides a quantification of the results of the FIG. 32. The results indicate that a use of hypoxic and positive pressure conditions to culture cells provided 45-times increased transfection efficiency over standard culture conditions. The p-value of the experiment was 1.68×10⁻¹⁸.

TABLE 2

GFP+ Cell

| | | Low Oxygen and Positive |
|---|---|---|
| Cell Count | Standard | Pressure |
| 5000 | 0/12 | 1/12 |
| 10000 | 0/12 | 12/12 |
| 50000 | 1/12 | 12/12 |
| 100000 | 4/12 | 12/12 |

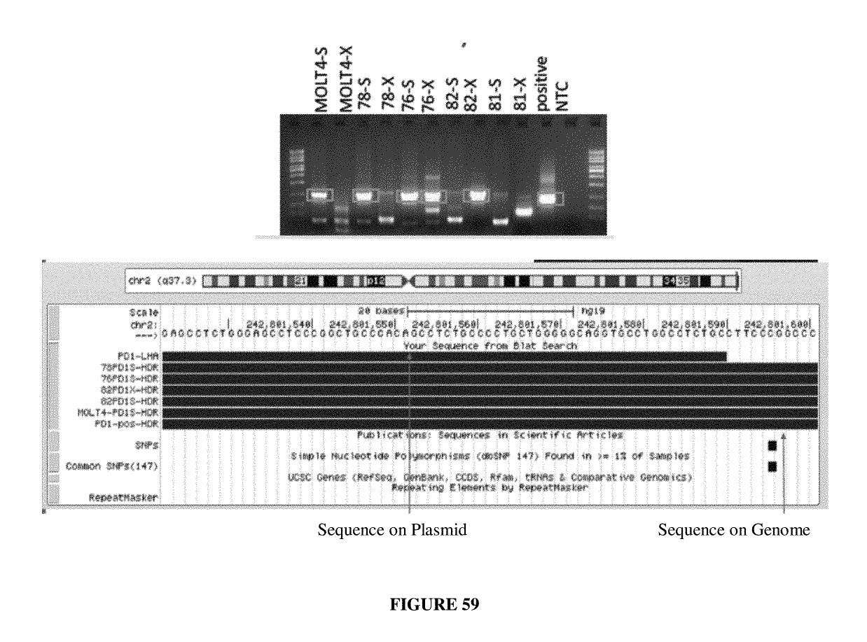

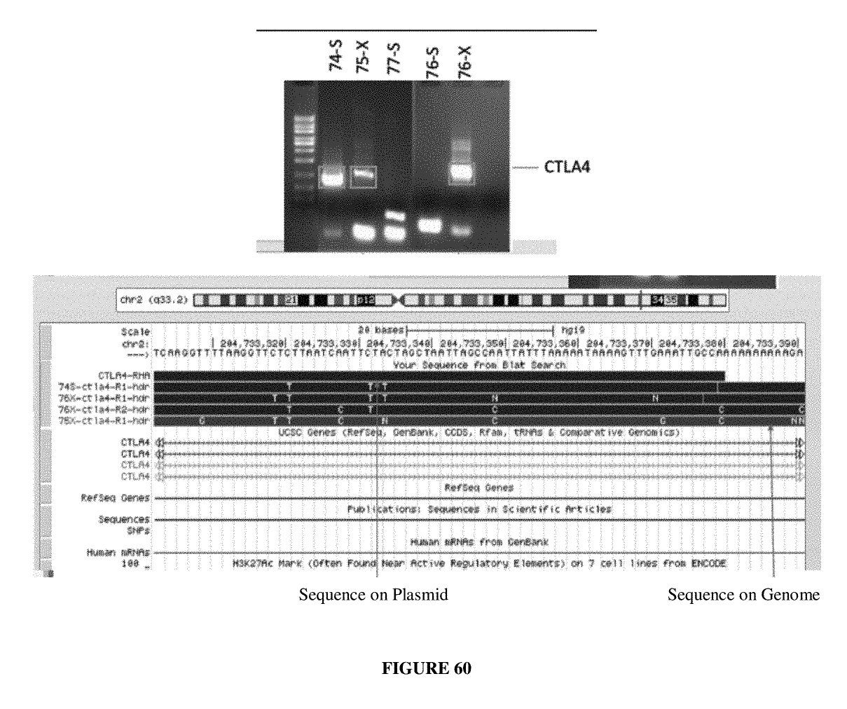

FIGS. 59-60 provide molecular confirmation of the genome editing experiments performed above. FIG. 59 provides data relating to the PD1 knockout, and FIG. 60 provides data relating to the CTLA4 knockout. Both figures show that the highlighted bands in the DNA gels (upper panels) were extracted and used as a template for sequencing. For FIG. 59, genome editing was confirmed for Donors 76, 78, 82, and MOLT4. The PD1-pos-HDR was a positive control from previous MOLT4 knockout cells. For FIG. 60, genome editing was confirmed for Donors 74, 75, and 76.

### Example 5: Reprogramming of Cells Using a Method of the Invention

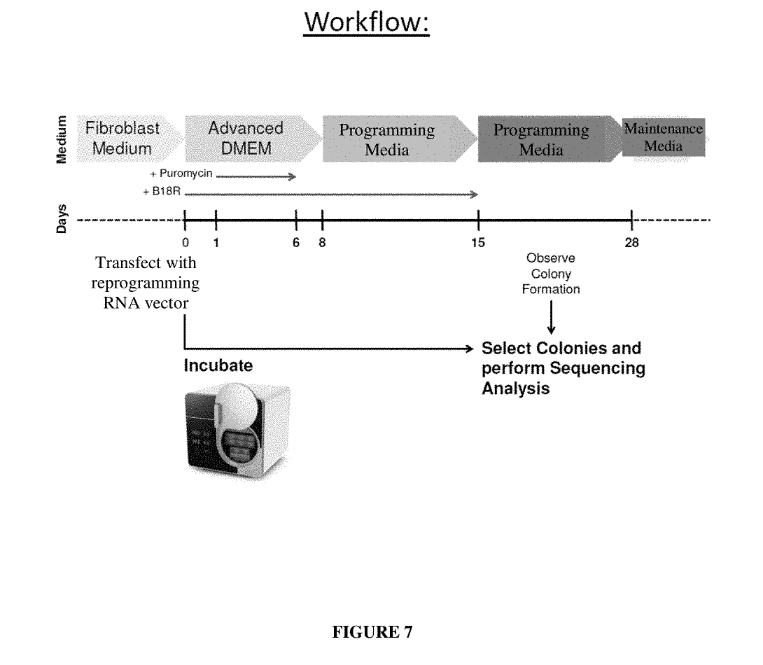

FIG. 7 depicts a workflow of a stem cell reprogramming experiment using a method of the invention. Prior to initiation of the reprogramming process, human fibroblasts were plated in fibroblast medium until the cells reached a desired confluency. The cells were cultured either under standard conditions or under hypoxic and positive pressure conditions. The fibroblasts were then transfected with a RNA vector encoding Oct4, KLF-4, SOX2, GLIS1, and c-MYC, and a puromycin resistance gene. After 5 days of puromycin selection post-transfection, the cells were cultured in reprogramming media for the remainder of the reprogramming induction phase until the induced pluripotent stem cell (iPSC) colonies emerged. Recombinant B18R Protein was also added during the first 2 weeks after transfection to inhibit the interferon response and increase cell viability. After about 20 days, the iPSC colonies were isolated and propagated in maintenance media. The maintenance media was a complete, serum-free media designed for the feeder-free maintenance and expansion of stem cells. The maintenance media contained recombinant human basic fibroblast growth factor and recombinant human transforming growth factor β. The reprogramming media was a complete, xeno-free defined reprogramming media designed for generating iPSCs under feeder-free conditions.

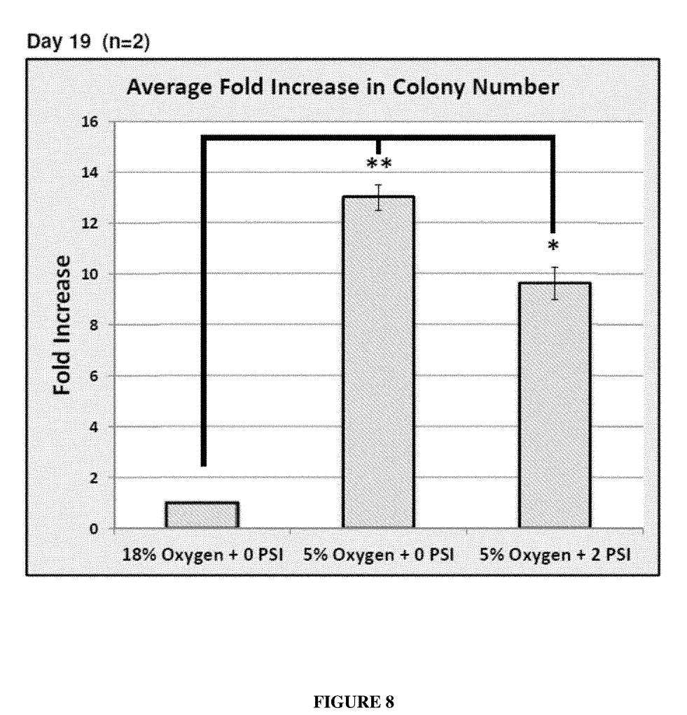

The colonies formed from standard or hypoxic and high pressure conditions were assessed for various markers. FIG. 8 shows that by day 19, the average fold increase in colony number was higher in cells grown under hypoxic and either standard or high pressure conditions as compared to standard conditions (18% oxygen and 0 PSI). For example, cells grown under 5% oxygen and 0 PSI had about a 13-fold increase in colony number as compared to cells grown in 18% oxygen and 0 PSI, which increase was statistically significant. Further, cells grown under 5% oxygen and 2 PSI had about a 10-fold increase in colony number as compared to cells grown in 18% oxygen and 0 PSI, which increase was statistically significant.

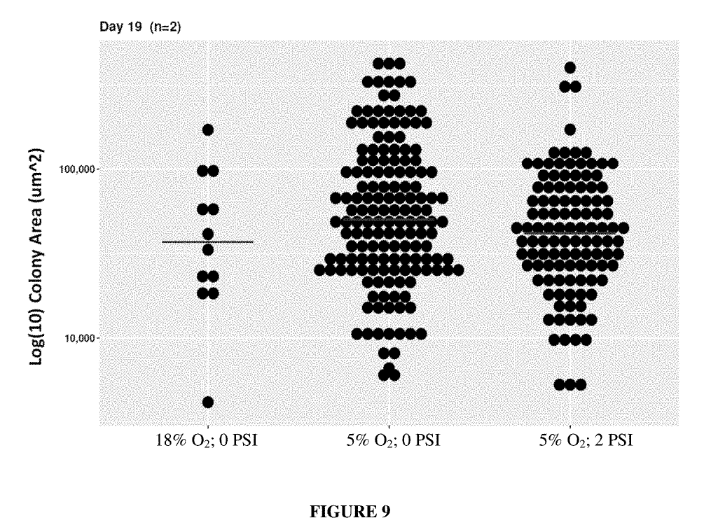

FIG. 9 depicts the frequency distribution of colony area between cells cultured in 18% oxygen; 0 PSI, 5% oxygen; 0 PSI, and 5% oxygen; 2 PSI conditions. The graph illustrates that the colony area (measured in μm²), was greater in cells grown under hypoxic and either standard or positive pressure conditions.

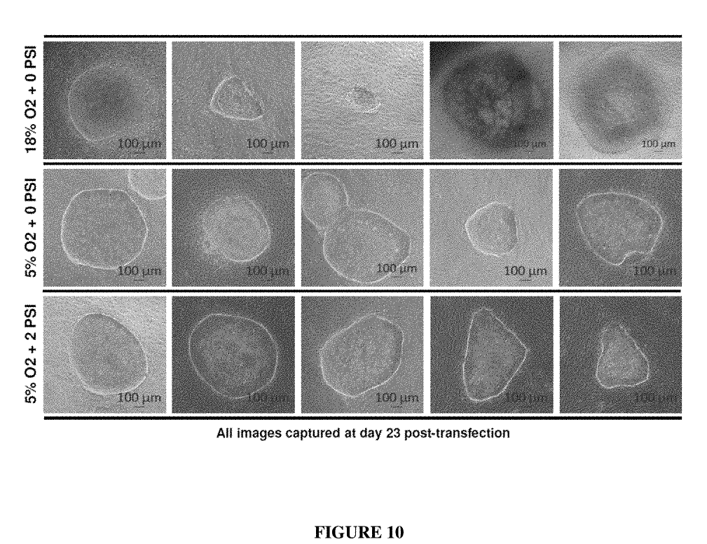

FIG. 10 shows microscopy images of the morphology of cells grown at 18% oxygen; 0 PSI, 5% oxygen; 0 PSI, and 5% oxygen; 2 PSI conditions. The images show that cells grown under hypoxic and either standard or high pressure conditions had a greater cell area than those cells grown under standard conditions.

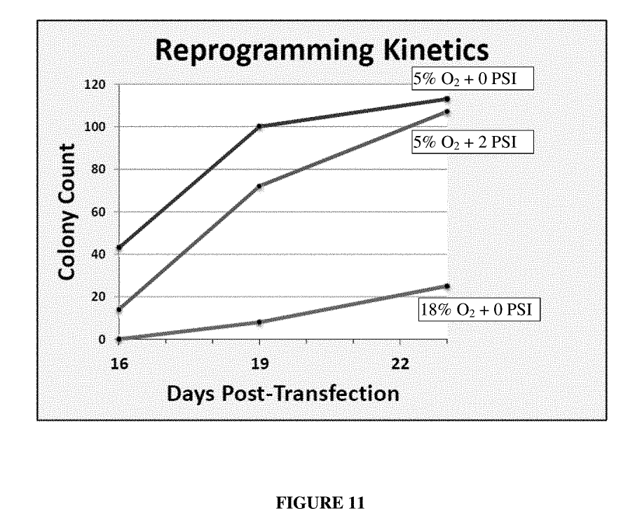

FIG. 11 shows the reprogramming kinetics of cells grown under 18% oxygen; 0 PSI (standard), 5% oxygen; 0 PSI, and 5% oxygen; 2 PSI conditions. The graph shows that cells grown under hypoxic and either standard or positive pressure conditions had a higher rate of reprogramming, as indicated by the increase in stem cell colony count per days post-transfection, as compared to cells grown under standard conditions.

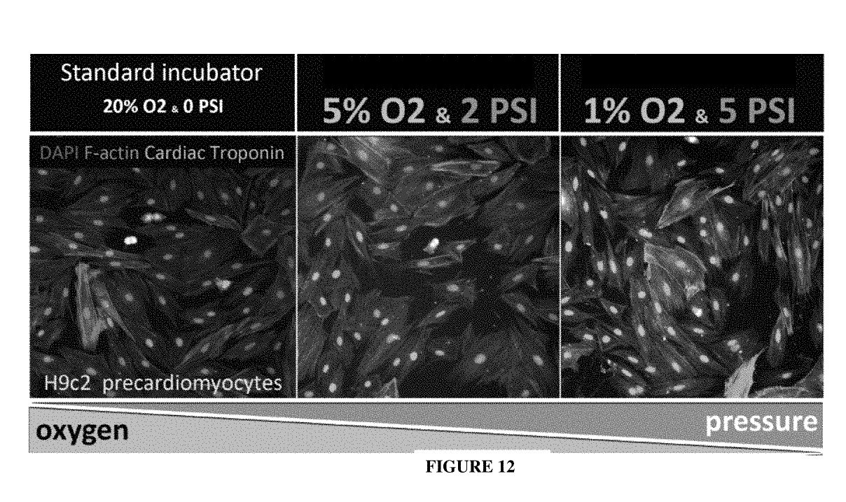

FIG. 12 shows the effect of hypoxia and positive pressure conditions on pre-cardiomyocyte differentiation and morphology. H9c2 pre-cardiomyocytes were cultured under 20% oxygen; 0 PSI (standard conditions), 5% oxygen; 2 PSI, and 1% oxygen; 5 PSI. The cells were then stained with DAPI to identify the nuclei of the cells, F-actin to visualize the individual cells, and cardiac troponin, a marker of cardiomyocytes. The results indicated that with decreasing oxygen levels and increasing pressure levels, the cells expressed increasing levels of cardiac troponin as shown by the more intense staining in the cytoplasm of the cells.

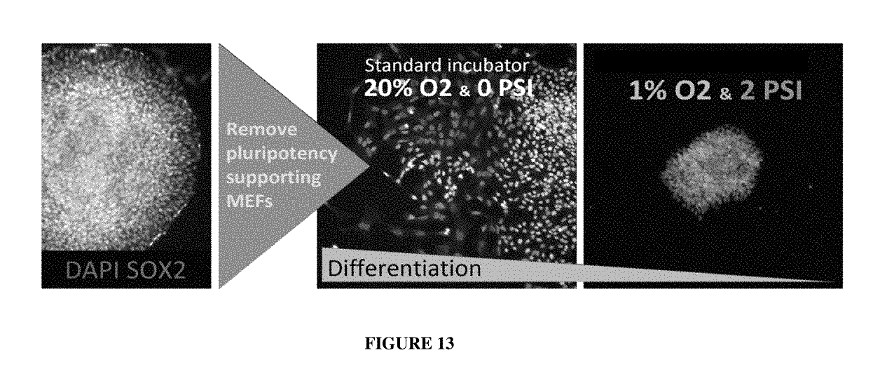

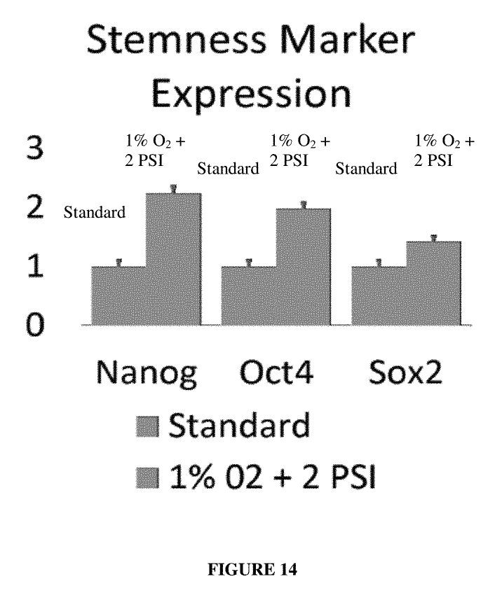

FIG. 13 shows staining of fibroblasts with DAPI to identify the nuclei of the cells and Sox2, a stem cell marker. After removal of pluripotency supporting mouse embryonic fibroblasts (MEFs), the cells grown under standard conditions (20% oxygen and 0 PSI) differentiated, while the cells grown under 1% oxygen and 2 PSI conditions maintained their dedifferentiated state. The differentiation was indicated by the Sox2 staining and the morphology of the cells after culture in the varying oxygen and pressure conditions. The cells were assessed for other stem cells markers by qPCR as shown in FIG. 14.

FIG. 14 shows that the cells grown under 1% oxygen and 2 PSI had higher levels of Nanog, Oct4, and Sox2 as compared to cells grown under standard conditions.

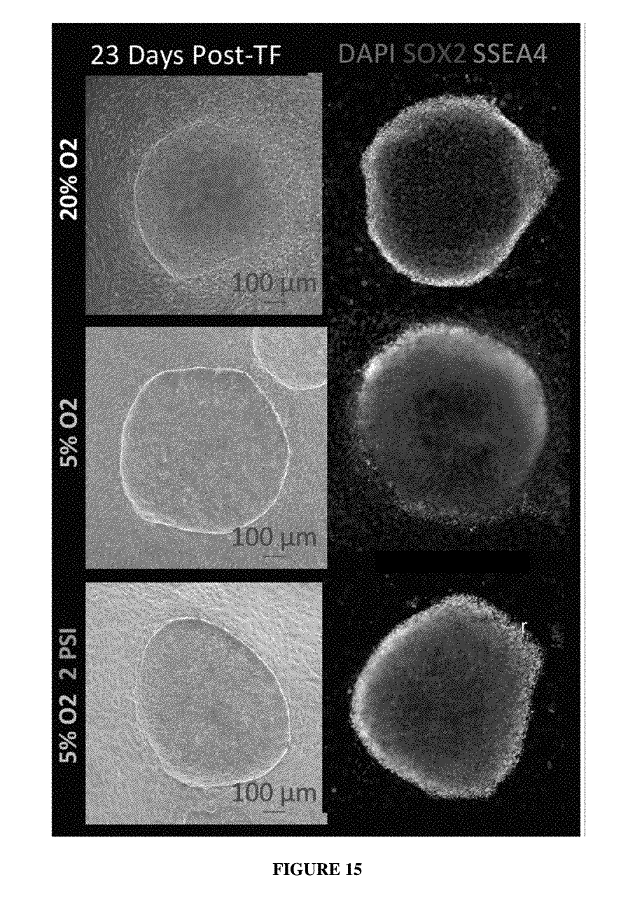

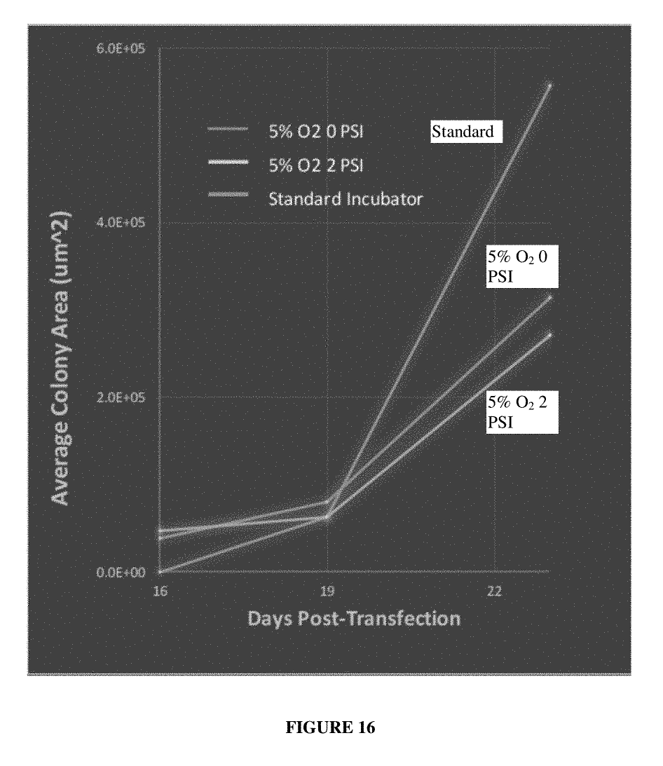

FIG. 15 shows staining of fibroblasts 23 days post-transfection of the reprogramming RNA vector as described above under 20% oxygen; 0 PSI (standard conditions); 5% oxygen; 0 PSI, and 5% oxygen; 2 PSI. The cells were stained with DAPI, Sox2, and SSEA4, the latter two of which are stem cells markers. Cells of the same size from each condition were analyzed for expression of SSEA4, a human embryonic stem cell marker. The results indicated that the cells grown under hypoxic and either standard or high pressure conditions showed greater staining for SSEA4, as indicated by the more intense staining around the periphery of the cells in FIG. 15. FIG. 16 shows the average colony area over the experimental period for the aforementioned conditions.

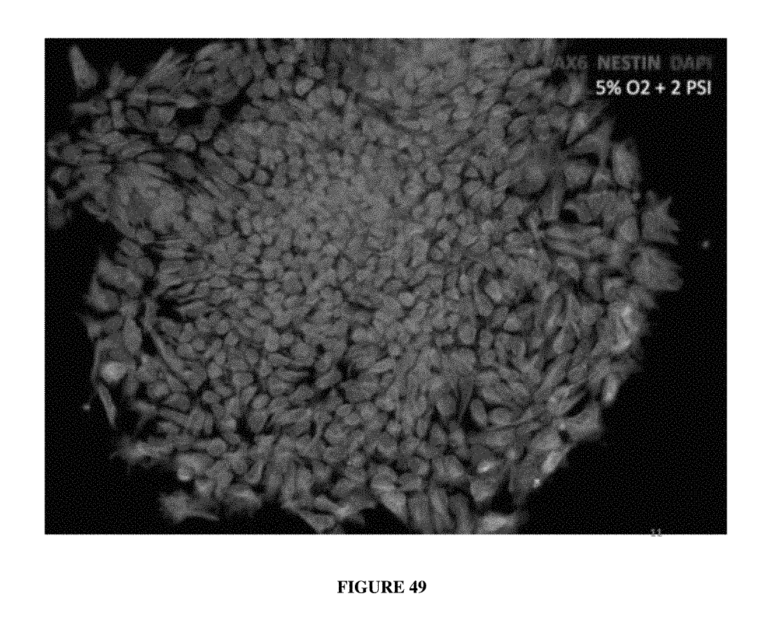

FIG. 49 shows induction of neural precursor markers, PAX6 and NESTIN, in iPSCs after two weeks in culture under 5% O₂ and 2 PSI in stem cell maintenance media.

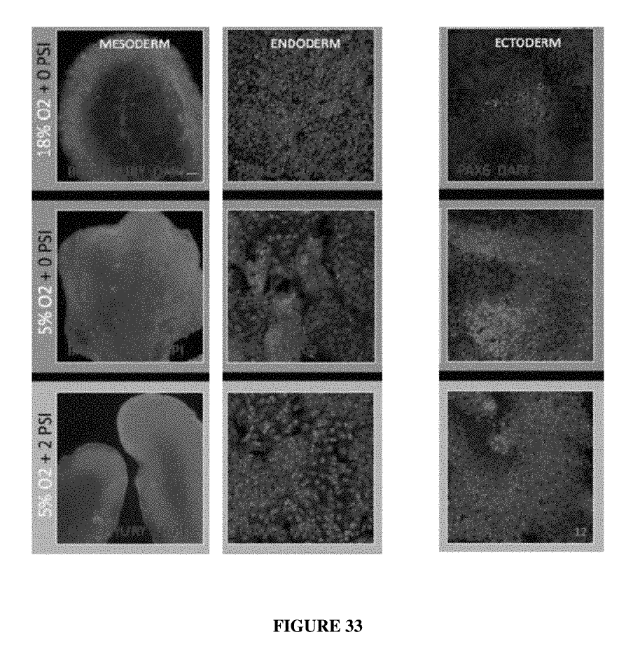

FIG. 33 shows that a combination of low oxygen and positive pressure enhances ectoderm commitment in defined medium, while causing changes in colony morphology to more mesoderm-like morphology. The directed differentiation of iPSCs to all three germ layers was performed using defined medium under the indicated cell culture conditions. Each experiment was performed in triplicate using independent iPSC reprogrammed cells lines at passage 5. The results indicate that the brachyury (mesoderm indicator) was more prominent along the edges of the cells under culture at hypoxic and high pressure conditions. FOXA2 (endoderm indicator) was more prominent in cells under culture at hypoxic and high pressure conditions. Finally, PAX6 (ectoderm indicator) was more highly expressed in the cells under grown under low oxygen and high pressure conditions.

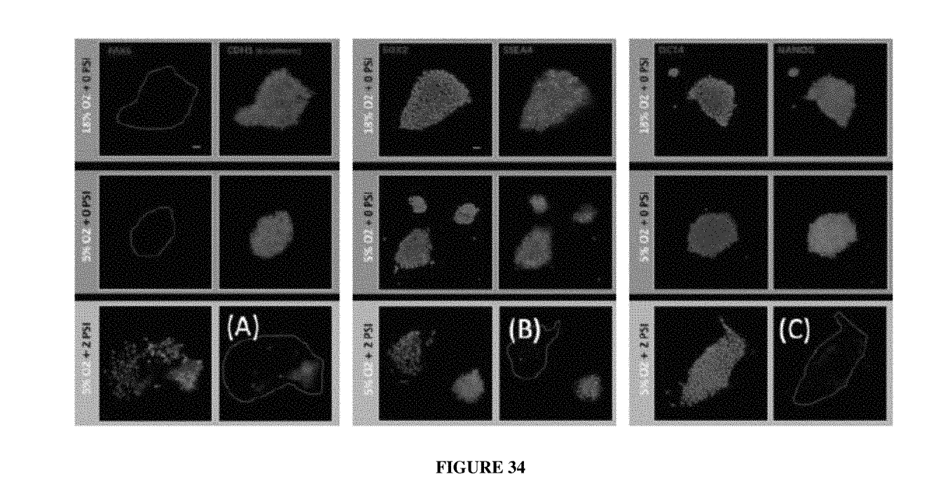

FIG. 34 shows that induction of PAX6 in iPSCs was accompanied by a loss of E-cadherin (indicated by CDH1 staining) under conditions of low oxygen and positive pressure (A; left panel is PAX6 staining; right panel is CDH1 staining). Additionally, SOX2, SSEA4, and NANOG staining decreased at low oxygen and positive pressure conditions, while OCT4 remained fairly constant throughout the three experimental conditions (B; left panel is SOX2 staining, right panel is SSEA4 staining. C; left panel is OCT4 staining, right panel is NANOG staining).

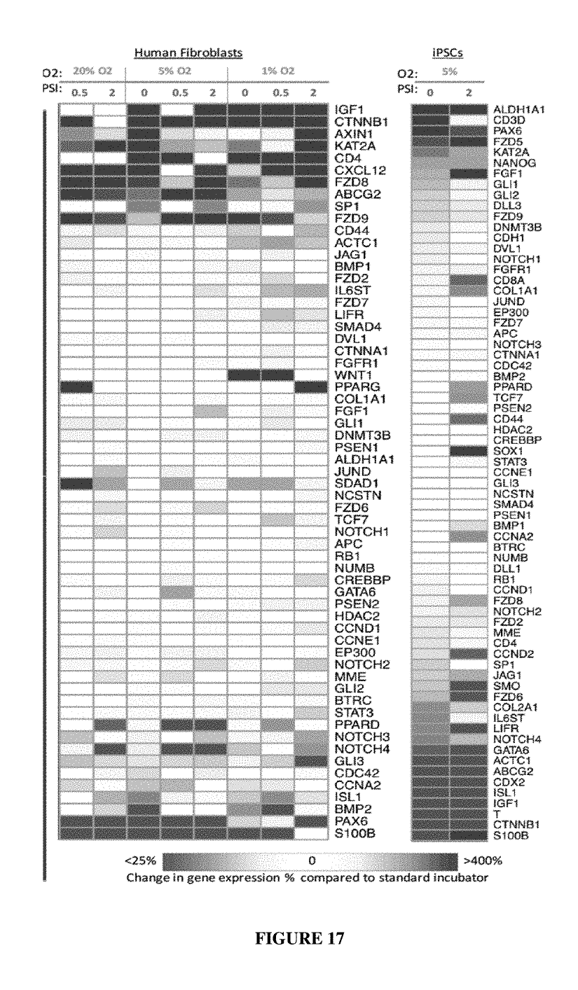

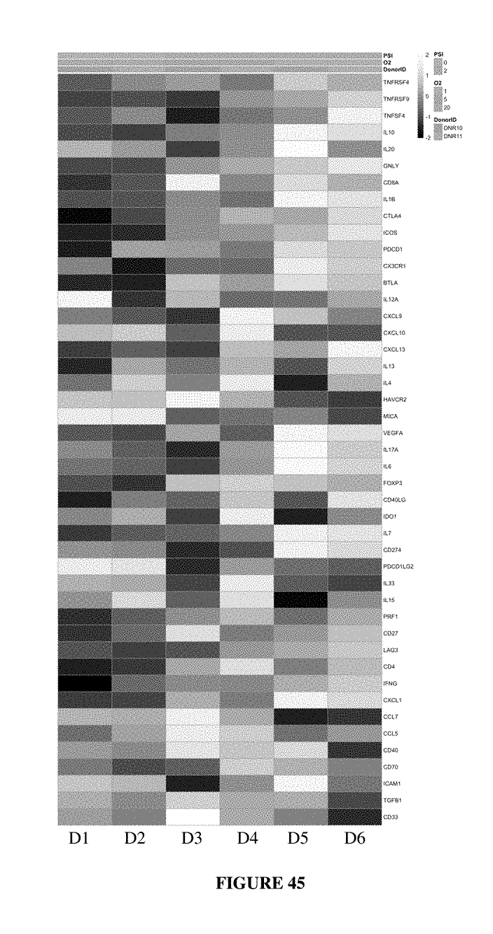

FIG. 17 shows the gene expression profiles as a function of oxygen concentration and pressure as compared to standard incubation conditions. The results indicated that oxygen concentration and pressure had an effect on the gene expression profile of several iPSC marker genes of interest. FIG. 45 shows the effect that various oxygen and pressure conditions had on the gene expression of immunotherapeutic targets in donor PBMCs. The cell culture conditions were created to mimic the vasculature and tumor microenvironments. The sample size for each condition was 12. The results of FIG. 45 are quantified (on a logarithmic scale) in TABLE 3 below.

TABLE 3

| | Donor 1 | Donor 2 | Donor 3 | Donor 4 | Donor 5 | Donor 6 |
|---|---|---|---|---|---|---|
| TNFRSF4 | 6.619909 | 6.866825 | 6.948752 | 6.77406 | 7.199577 | 7.044961 |
| TNFRSF9 | 7.11055 | 7.182824 | 7.046122 | 7.770995 | 7.907799 | 8.281298 |
| TNFSF4 | 6.959052 | 7.238442 | 6.69186 | 7.131686 | 7.270319 | 7.912743 |
| IL10 | 7.571088 | 7.472796 | 8.289168 | 8.460592 | 10.14985 | 9.663655 |
| IL20 | 3.923793 | 3.7464 | 3.066 | 3.650412 | 4.757204 | 3.658009 |
| GNLY | 11.58694 | 11.58746 | 11.92236 | 12.00283 | 12.14272 | 12.32933 |
| CD8A | 10.58403 | 10.74872 | 11.73939 | 11.03479 | 11.5482 | 11.26316 |
| IL1B | 12.26734 | 12.36899 | 13.85733 | 13.13851 | 17.2512 | 16.41079 |
| CTLA4 | 7.218212 | 7.548015 | 7.96451 | 8.191502 | 8.123371 | 8.438327 |
| ICOS | 8.459099 | 8.514474 | 9.260944 | 9.310994 | 9.568987 | 9.989088 |
| PDCD1 | 5.298568 | 5.887669 | 5.870134 | 5.689587 | 6.187069 | 6.070108 |
| CX3CR1 | 6.848331 | 6.521741 | 6.770097 | 6.755467 | 7.244198 | 7.098837 |
| BTLA | 6.550678 | 6.491376 | 7.731419 | 7.463986 | 8.060028 | 7.759103 |
| IL12A | 5.238696 | 4.780255 | 5.055161 | 4.893 | 4.910049 | 5.020782 |
| CXCL9 | 4.88878 | 4.39225 | 4.078183 | 6.442779 | 5.653539 | 4.950788 |
| CXCL10 | 6.477897 | 6.717857 | 5.181365 | 7.381816 | 4.957034 | 4.986273 |
| CXCL13 | 8.705631 | 8.945058 | 8.734548 | 9.69251 | 9.49176 | 10.32852 |
| IL13 | 3.67695 | 4.39225 | 4.078183 | 4.453252 | 3.883496 | 4.668556 |
| IL4 | 4.262466 | 4.885367 | 4.356257 | 5.157029 | 3.736401 | 4.668556 |
| HAVCR2 | 9.170135 | 9.146919 | 9.582496 | 9.055441 | 8.495221 | 8.383747 |
| MICA | 9.5119 | 9.485452 | 8.457327 | 8.581001 | 8.713503 | 8.310856 |
| VEGFA | 10.82009 | 10.66857 | 11.57516 | 10.85657 | 12.58646 | 12.20046 |
| IL17A | 4.394877 | 4.019712 | 3.658276 | 4.555729 | 5.5696 | 4.950788 |
| IL6 | 10.67563 | 10.36244 | 9.83626 | 11.30245 | 13.4968 | 12.44336 |
| FOXP3 | 7.649995 | 7.533283 | 8.282547 | 8.390411 | 8.306643 | 8.195861 |
| CD40LG | 6.142305 | 6.637095 | 6.475081 | 7.027289 | 6.392924 | 7.242464 |
| IDO1 | 10.16081 | 10.41552 | 9.648315 | 11.00966 | 9.463818 | 10.17725 |
| IL7 | 5.298568 | 5.509989 | 5.591429 | 5.876287 | 6.829225 | 6.680652 |
| CD274 | 9.902727 | 9.830315 | 8.97929 | 9.246269 | 10.94272 | 10.59646 |
| PDCD1LG2 | 9.004413 | 8.817339 | 6.071134 | 7.61544 | 6.963616 | 6.700756 |
| IL33 | 7.31835 | 7.105141 | 3.066 | 10.27267 | 3.883496 | 3.066 |
| IL15 | 7.450272 | 7.687581 | 7.290701 | 7.714615 | 7.036545 | 7.434076 |
| PRF1 | 9.608044 | 9.799925 | 10.05587 | 10.23814 | 9.88916 | 10.17636 |
| CD27 | 7.499824 | 7.739823 | 8.341071 | 7.842887 | 7.983301 | 8.152978 |
| LAG3 | 7.703818 | 7.605478 | 7.701923 | 8.135379 | 8.251017 | 8.432363 |
| CD4 | 9.963468 | 10.03466 | 10.39339 | 10.6011 | 10.25358 | 10.45002 |
| IFNG | 3.066 | 4.019712 | 4.356257 | 4.339943 | 4.701601 | 5.020782 |
| CXCL1 | 13.46389 | 13.51905 | 14.50338 | 14.0498 | 15.36751 | 14.96332 |
| CCL7 | 10.178 | 10.03205 | 11.89334 | 9.941749 | 6.974265 | 7.373004 |
| CCL5 | 11.94975 | 12.08067 | 12.31532 | 12.19939 | 11.9531 | 12.05798 |
| CD40 | 8.757388 | 8.690513 | 9.051137 | 8.895786 | 8.983975 | 8.414324 |
| CD70 | 5.700041 | 5.446335 | 5.546533 | 6.159697 | 5.988991 | 5.736881 |
| ICAM1 | 10.56529 | 10.50489 | 9.923909 | 10.34862 | 10.86652 | 10.23831 |
| TGFB1 | 11.26375 | 11.17533 | 11.36419 | 11.25554 | 11.26814 | 11.0061 |
| CD33 | 7.582629 | 7.291983 | 9.093345 | 7.724167 | 7.278922 | 6.414203 |

TABLE 4 below shows the effect that high atmospheric pressure had on iPSCs grown under hypoxic conditions as assessed by digital PCR. The results indicate that that was a change in gene expression of various neuronal, bone, and cardiomyocyte factors. TABLE 4 shows relative gene expression changes with 1 being no change, and values above 1 indicating greater expression, and values below 1 indicating lower expression.

TABLE 4

| | iPSC | iPSC |
|---|---|---|
| | 5% O₂ + 0 PSI | 5% O₂ + 2 PSI |
| PAX6 | 13.167 | 116.833 |
| FABP7 | 3.083 | 7.488 |
| LEFTY2 | 3.047 | 1.526 |
| COL2A1 | 2.997 | 21.564 |
| FOXA2 | 2.761 | 1.674 |
| BMP2 | 2.682 | 2.071 |
| OTX2 | 2.179 | 2.513 |
| NCAM1 | 2.038 | 3.667 |
| CDH2 | 2.025 | 7.926 |
| GATA2 | 2.000 | 1.000 |
| FGFR1 | 1.994 | 1.683 |
| COL9A1 | 1.791 | 1.764 |
| ACTC1 | 1.784 | 7.826 |
| RUNX1 | 1.667 | 7.667 |
| COMMD3 | 1.656 | 4.008 |
| PTEN | 1.574 | 1.902 |
| COL1A1 | 1.568 | 2.147 |
| NES | 1.535 | 3.579 |
| ALDH2 | 1.444 | 2.235 |
| SOX2 | 1.438 | 1.472 |
| CDC42 | 1.387 | 1.239 |
| EP300 | 1.382 | 1.412 |
| FGF2 | 1.343 | 0.948 |
| REST | 1.323 | 1.651 |
| HSPA9 | 1.276 | 1.060 |
| GPI | 1.274 | 1.253 |
| GABRB3 | 1.264 | 0.821 |
| CCNA2 | 1.259 | 1.173 |
| MYBL2 | 1.258 | 1.165 |
| CDK1 | 1.229 | 1.233 |
| LEFTY1 | 1.225 | 0.360 |
| TCF3 | 1.218 | 1.439 |
| ALPL | 1.209 | 1.119 |
| DPPA3 | 1.208 | 0.531 |
| SFRP2 | 1.197 | 1.921 |
| GRB7 | 1.189 | 0.533 |
| PSMB4 | 1.167 | 0.997 |
| NAT1 | 1.151 | 0.792 |
| APC | 1.145 | 1.361 |
| GDF3 | 1.138 | 0.443 |
| REEP5 | 1.133 | 0.996 |
| LIN28A | 1.125 | 1.065 |
| BRIX1 | 1.111 | 0.957 |
| PSMB2 | 1.104 | 1.016 |
| NUMB | 1.090 | 1.090 |
| HPRT1 | 1.089 | 0.774 |
| AFP | 1.082 | 1.135 |
| CCNE1 | 1.073 | 0.977 |
| PODXL | 1.068 | 0.737 |
| GJA1 | 1.067 | 1.177 |
| PARD6A | 1.060 | 0.766 |
| KAT7 | 1.052 | 1.037 |
| KAT8 | 1.019 | 0.843 |
| POU5F1 | 0.982 | 0.548 |
| RAB7A | 0.973 | 1.062 |
| CDH1 | 0.954 | 0.365 |
| KAT2A | 0.946 | 0.996 |
| DNMT3B | 0.935 | 0.589 |
| CD9 | 0.894 | 0.398 |
| FOXD3 | 0.880 | 0.343 |
| DPPA2 | 0.876 | 0.425 |
| MYCN | 0.867 | 1.018 |
| KLF4 | 0.806 | 1.032 |
| MYC | 0.759 | 0.822 |
| TDGF1 | 0.757 | 0.291 |
| TBX3 | 0.750 | 1.250 |
| HDAC2 | 0.739 | 0.773 |
| ALDH1A1 | 0.708 | 1.542 |
| RUNX2 | 0.684 | 0.947 |
| EMX2 | 0.667 | 44.000 |
| ZFP42 | 0.606 | 0.464 |
| NODAL | 0.583 | 0.313 |
| GAL | 0.485 | 0.175 |
| NANOG | 0.485 | 0.182 |
| NR5A2 | 0.218 | 0.113 |
| FGF5 | | 1.500 |

### Example 6: Change in Gene Expression in Cancer Cells

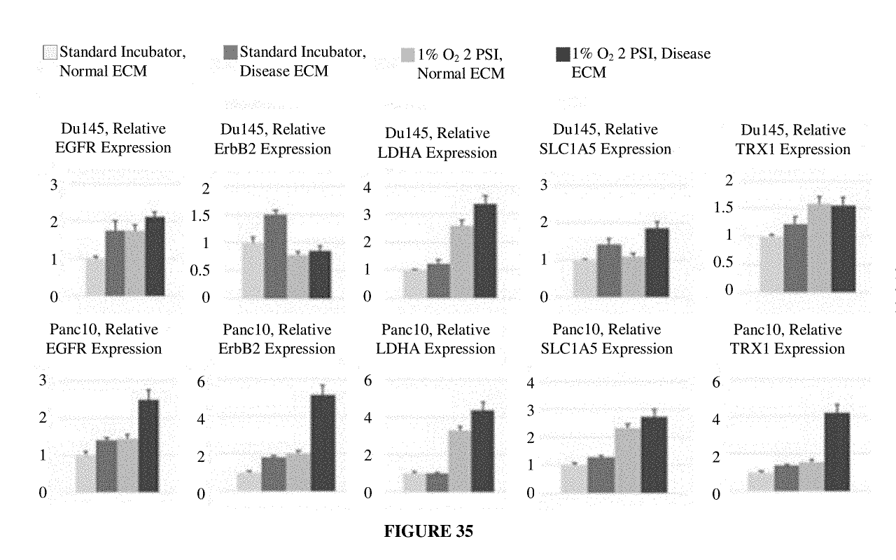

FIG. 35 show that different combinations of tumor (disease) extracellular matrix (ECM), low oxygen, and high pressure can alter the gene expression of EGFR and other metabolic regulators in DU145 (prostate cancer) and Panc10 (pancreatic cell lines). The markers measured included EGFR, ErbB2, LDHA, SLC1A5, and TRX1. The results indicate that, generally, gene expression increased with low oxygen, high pressure, and tumor ECM conditions. However, for ErbB2 in DU145 cells, the ErbB2 expression decreased compared to standard incubator conditions when the cells were cultured under hypoxic and high pressure conditions.

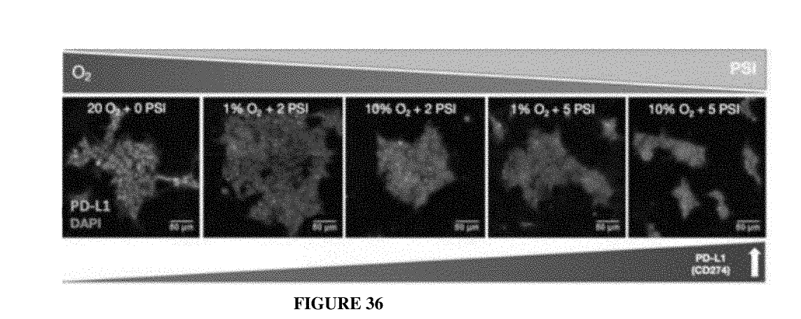

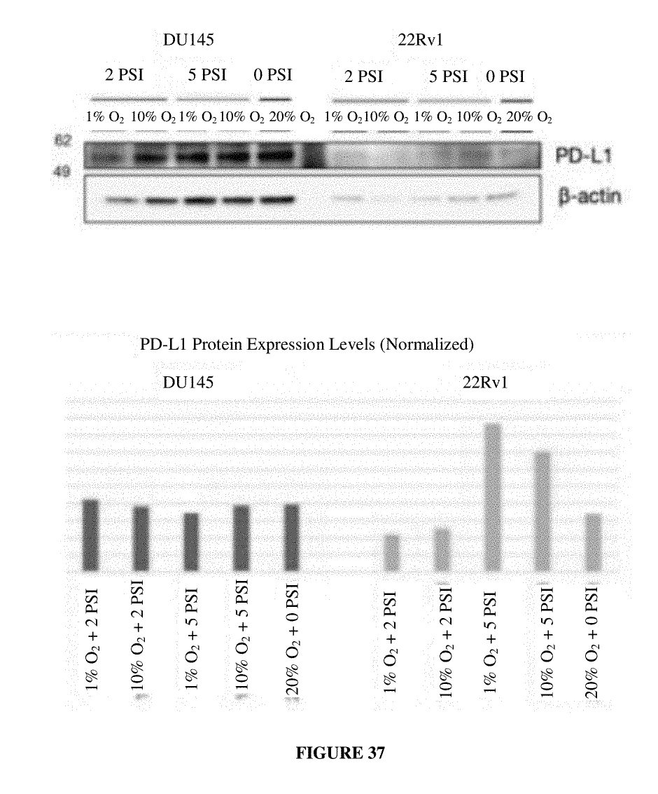

FIG. 36 shows that PDL1 expression increased in ARV7-positive, 22RV1 prostate cancer cells during low oxygen and positive pressure culturing conditions. The top panel of FIG. 37 provides a western blot showing increased PDL1 protein expression under various conditions of high pressure and hypoxia in both DU145 and 22Rv1 prostate cancer cells. The bottom panel of FIG. 37 provides a quantification of the western blot results normalized to actin. The results indicated that the change in PDL1 expression was more pronounced in the 22Rv1 prostate cancer cells, rather than the DU145 prostate cancer cells.

TABLES 5-10 below show the effect of varying oxygen and pressure on the gene expression profiles of various cancer targets when compared to traditional culturing approaches.

TABLE 5
LnCAP (2 PSI)

| 1% O₂ | 10% O₂ | 20% O₂ |
|---|---|---|
| VEGFA (higher) | EGFR (higher) | PRPF40A (lower) |
| GPI (higher) | GPI (higher) | SSSCA1 (lower) |
| HK2 (higher) | HSP90B1 (lower) | VEGFA (higher) |
| HSP90B1 (lower) | VEGFA (higher) | EGFR (higher) |
| EPAS1 (lower) | GPX1 (lower) | NFκB1 (lower) |
| EGFR (higher) | SPG7 (lower) | PARP2 (lower) |

TABLE 6
LnCAP (5 PSI)

| 1% O₂ | 10% O₂ | 20% O₂ |
|---|---|---|
| GPI (higher) | EGFR (higher) | NFκB1 (lower) |
| HK2 (higher) | HDAC3 (higher) | HDAC3 (higher) |
| ITGAV (lower) | PK3C2A (higher) | LAMA3 (lower) |
| EGFR (higher) | PLOD3 (higher) | PARP2 (lower) |
| VEGFA (higher) | GP1 (higher) | HSP90B1 (lower) |
| HDAC3 (higher) | CDK5 (lower) | SSSCA1 (higher) |

TABLE 7
PC-3 (2 PSI)

| 1% O₂ | 10% O₂ | 20% O₂ |
|---|---|---|
| ATF2 (higher) | ITGAV (lower) | SSSCA1 (lower) |
| HDAC3 (higher) | ATF2 (lower) | PEA15 (higher) |
| GP1 (higher) | PEA15 (lower) | CDK2 (higher) |
| PEA15 (lower) | IGF1R (lower) | ATF2 (higher) |
| HK2 (higher) | PLK4 (higher) | NFκB1 (lower) |
| HPRT1 (higher) | RHOB (lower) | ABCC1 (lower) |

TABLE 8
PC-3 (5 PSI)

| 1% O₂ | 10% O₂ | 20% O₂ |
|---|---|---|
| GP1 (higher) | PI3KC2a (lower) | P992CB (higher) |
| HK2 (higher) | CDC25A (lower) | CDK7 (lower) |
| PARP2 (lower) | PLK4 (lower) | HDAC3 (lower) |
| PLK1 (lower) | PRPF40A (lower) | ERBB2 (higher) |
| SSSCA1 (lower) | MAN2B1 (higher) | PEA15 (higher) |
| AURKA (lower) | HSP90B1 (higher) | HK2 (lower) |

TABLE 9
PANC-10.05 (2 PSI)

| 1% O₂ | 10% O₂ | 20% O₂ |
|---|---|---|
| GP1 (higher) | GP1 (higher) | CTSB (lower) |
| ITGAV (lower) | SPG7 (lower) | ITGAL (lower) |
| VEGFA (higher) | PARP2 (lower) | ITGAV (lower) |
| CDK7 (lower) | CDC25A (lower) | CDK2 (higher) |
| MAN2B1 (higher) | MMP1 (higher) | GPI (higher) |
| HRAS (lower) | COL12A1 (lower) | ENO1 (lower) |

TABLE 10
PANC-10.05 (5 PSI)

| 1% O₂ | 10% O₂ | 20% O₂ |
|---|---|---|
| GP1 (higher) | GP1 (higher) | RHOB (lower) |
| VEGFA (higher) | CDK5 (higher) | CTSB (lower) |
| ADM (higher) | CDC25A (lower) | BIRC5 (lower) |
| DR1 (lower) | PPP2CB (higher) | ITGAL (lower) |
| HK2 (higher) | TXN_ALT (higher) | PIK3C3 (lower) |
| NAA10 (lower) | TXNRD1 (higher) | ITGAV (lower) |

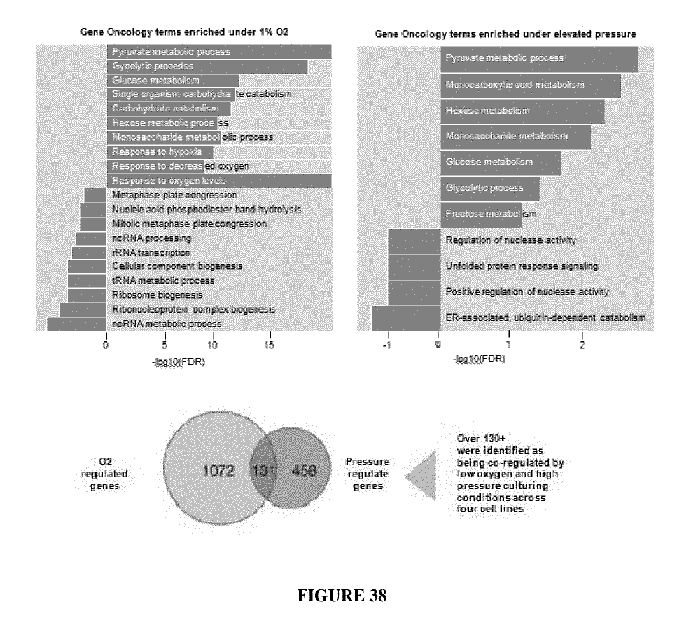

FIG. 38 shows identification of pressure and oxygen sensitive gene expression signatures in various cell lines. The results indicated that there was an enrichment of metabolic processes involved in cell survival. The results in the top panel of FIG. 38 were corrected for any false discovery rate. The top panel of FIG. 38 provides various gene ontology terms that were enriched under low oxygen or positive pressure conditions. The bars to the right of the 0 on the x-axis indicated upregulated genes, and the bars to the left of the 0 on the x-axis indicated downregulated genes. The bottom panel of FIG. 38 shows that over 130 genes were shown to be co-regulated by low oxygen and positive pressure conditions across various cell lines.

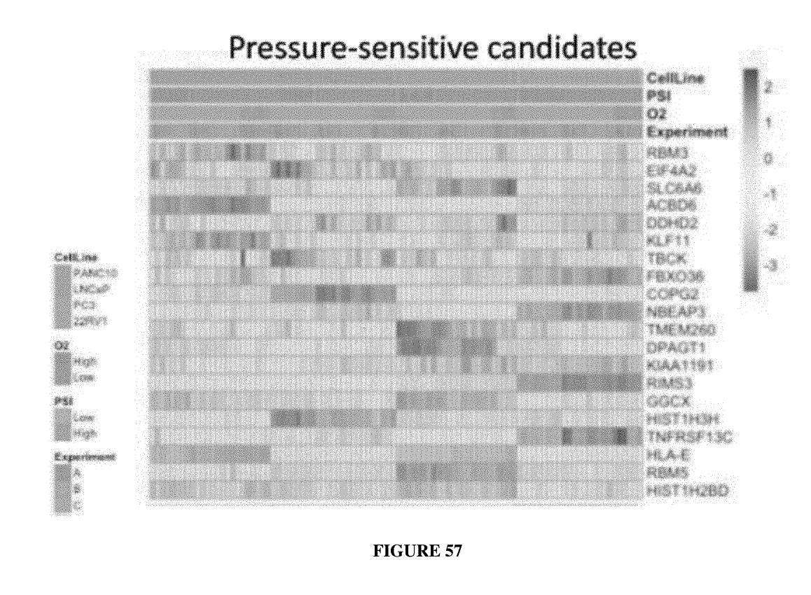

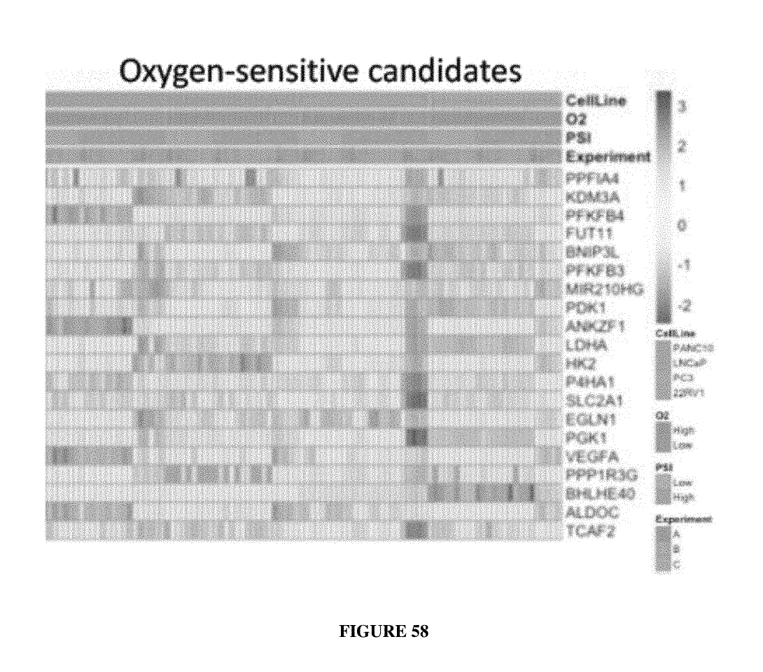

FIGS. 57 and 58 show the effect of various experimental conditions on various cells lines. FIG. 57 shows pressure-sensitive genes, while FIG. 58 depicts oxygen-sensitive genes. The cells lines tested for both figures was (from left to right) PANC10, LNCaP, PC3, and 22Rv1. Within each group of cell lines, the cells were exposed to high (>18%; left-most columns of cells) and low (<5%; right-most columns of cells) oxygen, low pressure (0 PSI; left-most columns of cells) and positive pressure (2 PSI; right-most columns of cells). TABLES 11-16 below provide the quantitative data for the heatmaps of FIGS. 57-58.

TABLE 11
Oxygen-Sensitive Candidates

| | O2 | PSI | Expt | CellLine | FUT11 | PFKFB4 | PPFIA4 | ANKZF1 | PFKFB3 | MIR210HG | BNIP3L | LDHA |
|---|---|---|---|---|---|---|---|---|---|---|---|---|
| LNCaP_25 | 20 | 0 | A | LNCaP | 148.8648 | 142.0982 | 8.458226 | 419.528 | 316.3376 | 10.14987 | 1283.959 | 11777.23 |
| 22RV1_3 | 20 | 0 | A | 22RV1 | 221.0045 | 97.77776 | 6.697107 | 420.5783 | 297.3515 | 18.7519 | 488.8888 | 10510.44 |
| PC3_3 | 20 | 0 | A | PC3 | 982.6681 | 450.2195 | 9.330973 | 306.7557 | 521.9513 | 20.4115 | 713.2363 | 19104.58 |
| DU145_3 | 20 | 0 | A | DU145 | 374.04 | 107.181 | 41.56 | 192.4884 | 1296.016 | 28.43579 | 3087.47 | 17411.45 |
| PANC10_12 | 20 | 0 | B | PANC10 | 261.2557 | 36.13111 | 2.779316 | 141.7451 | 390.4939 | 16.6759 | 922.733 | 18838.21 |
| LNCaP_1 | 20 | 0 | B | LNCaP | 157.7928 | 136.224 | 1.1352 | 407.5369 | 294.0168 | 21.5688 | 1683.502 | 11943.44 |
| 22RV1_12 | 20 | 0 | B | 22RV1 | 222.767 | 104.2549 | 6.237475 | 440.1875 | 308.3095 | 24.9499 | 619.2922 | 9452.448 |
| DU145_12 | 20 | 0 | B | DU145 | 446.3176 | 143.8638 | 32.85079 | 190.308 | 1184.894 | 52.10815 | 3061.92 | 23495.11 |
| PANC10_21 | 20 | 0 | C | PANC10 | 338.7363 | 30.03138 | 1.766552 | 150.1569 | 314.0046 | 18.10716 | 1237.028 | 22815.9 |
| LNCaP_10 | 20 | 0 | C | LNCaP | 159.8962 | 153.9 | 8.994158 | 376.7553 | 293.8092 | 17.98832 | 1323.141 | 11189.73 |
| 22RV1_21 | 20 | 0 | C | 22RV1 | 195.1418 | 84.6398 | 2.351106 | 391.4591 | 246.8661 | 28.21327 | 739.4227 | 8315.861 |
| PC3_25 | 20 | 0 | C | PC3 | 670.9391 | 423.8815 | 8.262797 | 356.1266 | 355.3003 | 13.22048 | 796.5337 | 17632.81 |
| DU145_21 | 20 | 0 | C | DU145 | 371.5608 | 119.1418 | 33.31931 | 209.0029 | 1529.659 | 36.34834 | 2347.497 | 18875.89 |
| PANC10_8 | 20 | 0 | A | PANC10 | 271.5097 | 21.81774 | 4.242339 | 199.996 | 283.6307 | 12.12097 | 994.5255 | 21212.91 |
| LNCaP_26 | 20 | 0 | A | LNCaP | 193.4126 | 109.4788 | 3.649293 | 385.0005 | 328.4364 | 18.24647 | 1591.092 | 13212.27 |
| 22RV1_6 | 20 | 0 | A | 22RV1 | 236.1916 | 112.9612 | 4.107679 | 465.1947 | 291.6452 | 15.4038 | 626.4211 | 9411.72 |
| PC3_6 | 20 | 0 | A | PC3 | 814.0391 | 385.4435 | 16.82201 | 409.5794 | 430.7898 | 22.67315 | 815.5019 | 21881.78 |
| DU145_6 | 20 | 0 | A | DU145 | 390.234 | 120.8689 | 34.53398 | 188.7858 | 1281.211 | 35.68512 | 3075.827 | 18655.26 |
| PANC10_15 | 20 | 0 | B | PANC10 | 266.3327 | 11.38174 | 1.138174 | 161.6207 | 357.3867 | 15.93444 | 1108.582 | 20638.51 |
| LNCaP_4 | 20 | 0 | B | LNCaP | 218.3846 | 168.5067 | 4.04416 | 413.8524 | 376.1069 | 22.91691 | 1480.163 | 12063.73 |
| 22RV1_15 | 20 | 0 | B | 22RV1 | 224.198 | 107.2251 | 3.899096 | 416.2285 | 296.3313 | 16.57116 | 611.1833 | 9461.156 |
| PC3_15 | 20 | 0 | B | PC3 | 1154.691 | 535.3124 | 26.03343 | 434.9752 | 612.328 | 71.04957 | 896.5263 | 22944.13 |
| DU145_15 | 20 | 0 | B | DU145 | 500.0377 | 221.7909 | 62.50471 | 264.1328 | 1630.163 | 87.70823 | 2433.651 | 26165.28 |
| PANC10_24 | 20 | 0 | C | PANC10 | 279.1962 | 23.19056 | 4.547169 | 155.9679 | 344.6754 | 12.27736 | 968.0923 | 18497.43 |
| LNCaP_13 | 20 | 0 | C | LNCaP | 191.8114 | 184.7509 | 5.883786 | 398.9207 | 303.6034 | 25.88866 | 1423.876 | 11905.25 |
| 22RV1_24 | 20 | 0 | C | 22RV1 | 222.8496 | 119.8661 | 5.064763 | 446.5433 | 254.0823 | 27.8562 | 882.9571 | 8997.552 |
| PC3_26 | 20 | 0 | C | PC3 | 752.2415 | 421.9328 | 6.159603 | 333.3885 | 432.7121 | 12.31921 | 878.5134 | 18287.86 |
| DU145_24 | 20 | 0 | C | DU145 | 315.9268 | 100.4485 | 42.12358 | 241.4005 | 1213.483 | 42.12358 | 1699.524 | 15059.18 |
| PANC10_9 | 20 | 0 | A | PANC10 | 308.4363 | 25.97358 | 0 | 220.7754 | 269.4759 | 12.98679 | 1042.19 | 19941.22 |
| LNCaP_27 | 20 | 0 | A | LNCaP | 187.4626 | 94.73918 | 0 | 417.2555 | 264.0603 | 20.15727 | 1408.993 | 12487.43 |
| 22RV1_9 | 20 | 0 | A | 22RV1 | 221.6903 | 108.7925 | 0 | 472.1183 | 279.1656 | 12.31613 | 665.071 | 9027.723 |
| PC3_9 | 20 | 0 | A | PC3 | 1047.944 | 516.1348 | 17.91357 | 383.4624 | 615.779 | 34.14775 | 1031.15 | 25294.52 |
| DU145_9 | 20 | 0 | A | DU145 | 525.8409 | 352.0274 | 81.40633 | 323.4251 | 1627.027 | 82.50642 | 3651.184 | 22824.57 |
| PANC10_18 | 20 | 0 | B | PANC10 | 294.2244 | 34.51794 | 9.862269 | 198.8891 | 295.8681 | 32.87423 | 1290.313 | 22765.4 |
| LNCaP_7 | 20 | 0 | B | LNCaP | 208.0811 | 123.3848 | 4.182534 | 367.0174 | 324.1464 | 13.59324 | 1846.589 | 13925.75 |
| 22RV1_18 | 20 | 0 | B | 22RV1 | 251.6665 | 100.153 | 3.852038 | 376.2157 | 290.1869 | 19.26019 | 735.7393 | 8831.439 |
| PC3_18 | 20 | 0 | B | PC3 | 876.1958 | 531.8069 | 48.03854 | 466.1768 | 508.1259 | 40.59595 | 883.6384 | 21119.36 |
| DU145_18 | 20 | 0 | B | DU145 | 358.0772 | 119.3591 | 30.744 | 233.2927 | 888.8633 | 55.15835 | 2062.561 | 18450.92 |
| PANC10_27 | 20 | 0 | C | PANC10 | 267.4496 | 25.71631 | 0 | 161.1555 | 330.8832 | 22.28747 | 865.7824 | 20482.18 |
| LNCaP_16 | 20 | 0 | C | LNCaP | 166.4081 | 147.7803 | 12.41851 | 409.811 | 294.3188 | 33.52999 | 1435.58 | 11798.83 |
| 22RV1_27 | 20 | 0 | C | 22RV1 | 220.7981 | 118.5017 | 4.051341 | 430.455 | 238.0163 | 16.20536 | 751.5237 | 8691.139 |
| PC3_27 | 20 | 0 | C | PC3 | 699.4571 | 410.7506 | 11.15457 | 398.9399 | 359.5708 | 14.43532 | 812.3151 | 17792.19 |
| DU145_27 | 20 | 0 | C | DU145 | 996.1885 | 0 | 110.6876 | 0 | 1217.564 | 221.3752 | 1771.002 | 19591.71 |
| PC3_PSI_1 | 20 | 0 | A | PC3 | 1664.168 | 254.2092 | 11.80753 | 548.0084 | 587.5984 | 77.7908 | 2127.44 | 18376.69 |
| PC3_PSI_2 | 20 | 0 | B | PC3 | 1500.024 | 210.9101 | 5.913367 | 488.1813 | 424.4483 | 96.585 | 2270.733 | 21171.83 |
| PC3_PSI_3 | 20 | 0 | C | PC3 | 1481.33 | 225.2641 | 12.73578 | 491.1234 | 602.5615 | 61.29093 | 2450.841 | 18856.11 |
| PANC10_4 | 10 | 2 | A | PANC10 | 388.3242 | 57.908 | 13.62541 | 170.3176 | 530.2556 | 40.87623 | 1196.765 | 12772.69 |
| PC3_2 | 10 | 2 | A | PC3 | 1127.084 | 485.2885 | 36.79082 | 395.9393 | 637.1236 | 50.80638 | 873.0521 | 21860.76 |
| DU145_2 | 10 | 2 | A | DU145 | 414.3253 | 80.87098 | 69.79276 | 168.3889 | 1087.881 | 86.41008 | 4175.38 | 18361.04 |
| PANC10_11 | 10 | 5 | B | PANC10 | 246.0487 | 33.96941 | 4.590461 | 169.8471 | 377.3359 | 6.426645 | 943.7988 | 18659.31 |
| LNCaP_2 | 10 | 5 | B | LNCaP | 247.6243 | 405.2033 | 14.06956 | 419.2729 | 478.3651 | 66.12694 | 2207.514 | 15593.29 |
| 22RV1_11 | 10 | 5 | B | 22RV1 | 211.1379 | 131.6928 | 6.441494 | 396.5097 | 334.242 | 19.32448 | 704.9857 | 10325.71 |
| DU145_11 | 10 | 5 | B | DU145 | 424.3177 | 102.2594 | 22.33251 | 202.168 | 983.8058 | 75.22529 | 3838.841 | 24570.46 |
| PANC10_19 | 20 | 2 | C | PANC10 | 234.8746 | 38.70092 | 6.672573 | 146.7966 | 334.9632 | 33.36287 | 914.1425 | 20149.84 |
| PANC10_20 | 20 | 5 | C | PANC10 | 253.3356 | 45.50203 | 5.53403 | 153.1082 | 348.029 | 33.20418 | 1054.54 | 21815.15 |
| LNCaP_11 | 20 | 2 | C | LNCaP | 161.5547 | 163.0369 | 4.446461 | 339.4132 | 250.484 | 34.08953 | 1551.815 | 10816.76 |
| LNCaP_12 | 20 | 5 | C | LNCaP | 160.2608 | 163.7447 | 0 | 369.2966 | 280.4564 | 27.87144 | 1477.186 | 10847.22 |
| 22RV1_19 | 20 | 2 | C | 22RV1 | 197.8738 | 119.4952 | 6.424474 | 409.8815 | 267.2581 | 23.12811 | 1029.201 | 8685.889 |
| PC3_19 | 20 | 5 | C | PC3 | 670.3506 | 441.7176 | 16.96196 | 342.0661 | 474.228 | 29.68342 | 804.9861 | 18424.92 |
| PC3_22 | 20 | 2 | C | PC3 | 616.4342 | 412.4344 | 10.13664 | 311.7016 | 456.1486 | 35.47823 | 866.0488 | 20432.29 |
| DU145_19 | 20 | 2 | C | DU145 | 283.305 | 110.0012 | 31.13242 | 176.4171 | 1270.203 | 53.96286 | 2028.796 | 17877.27 |
| DU145_20 | 20 | 5 | C | DU145 | 272.9211 | 81.08527 | 29.66534 | 288.7427 | 812.8303 | 55.3753 | 2099.317 | 18893.86 |
| LNCaP_23 | 10 | 2 | A | LNCaP | 210.4404 | 134.5439 | 3.449843 | 322.5603 | 260.4631 | 36.22335 | 1540.355 | 12221.07 |
| 22RV1_5 | 10 | 2 | A | 22RV1 | 231.5576 | 109.639 | 8.771119 | 511.3563 | 292.0783 | 16.66513 | 735.0198 | 9873.649 |
| PC3_5 | 10 | 2 | A | PC3 | 932.2809 | 406.043 | 18.49152 | 375.2238 | 530.8608 | 29.27824 | 976.1983 | 24877.26 |
| DU145_5 | 10 | 2 | A | DU145 | 531.7783 | 186.5714 | 121.7204 | 222.4889 | 1420.736 | 130.6997 | 4202.346 | 19979.1 |
| PANC10_14 | 10 | 5 | B | PANC10 | 244.5717 | 46.2249 | 8.404527 | 179.0164 | 401.7364 | 42.02264 | 1121.164 | 21281.94 |
| LNCaP_5 | 10 | 5 | B | LNCaP | 236.4201 | 149.1802 | 3.489596 | 343.7252 | 305.3396 | 28.78916 | 2145.229 | 14346.6 |
| 22RV1_14 | 10 | 5 | B | 22RV1 | 212.7266 | 123.0226 | 4.271619 | 423.7446 | 325.4974 | 22.21242 | 609.1329 | 10182.69 |
| PC3_14 | 10 | 5 | B | PC3 | 1372.075 | 660.5132 | 42.33261 | 462.546 | 750.1588 | 115.1696 | 1169.127 | 29175.88 |
| DU145_14 | 10 | 5 | B | DU145 | 718.0632 | 286.0285 | 113.6933 | 319.5381 | 1571.362 | 183.1061 | 3811.719 | 26951.31 |
| PANC10_22 | 20 | 2 | C | PANC10 | 241.9556 | 31.37496 | 1.898129 | 153.1902 | 385.2086 | 11.50043 | 835.9584 | 16977.98 |
| PANC10_23 | 20 | 5 | C | PANC10 | 224.4627 | 34.78357 | 1.086987 | 107.6117 | 385.3367 | 7.608906 | 980.4619 | 18153.76 |
| LNCaP_14 | 20 | 2 | C | LNCaP | 185.8022 | 182.617 | 0 | 425.7525 | 285.6046 | 35.03699 | 1472.615 | 12031.49 |
| LNCaP_15 | 20 | 5 | C | LNCaP | 210.3339 | 161.9813 | 3.626446 | 388.0297 | 233.3014 | 20.54986 | 1646.407 | 13451.7 |
| 22RV1_22 | 20 | 2 | C | 22RV1 | 185.5875 | 131.6668 | 7.523819 | 500.334 | 259.5718 | 35.11116 | 852.6995 | 8938.298 |
| 22RV1_23 | 20 | 5 | C | 22RV1 | 195.4212 | 109.9976 | 5.850934 | 439.9902 | 320.6312 | 19.89317 | 788.7058 | 9155.541 |
| PC3_20 | 20 | 5 | C | PC3 | 961.2017 | 488.5887 | 13.31304 | 398.7256 | 536.5156 | 34.61391 | 896.6334 | 22240.1 |
| PC3_23 | 20 | 2 | C | PC3 | 975.2113 | 431.7469 | 17.56259 | 385.4012 | 456.1394 | 34.63732 | 887.8863 | 22027.38 |
| DU145_22 | 20 | 2 | C | DU145 | 352.0325 | 123.6235 | 72.99672 | 243.7148 | 1377.519 | 65.93252 | 2081.584 | 15409.37 |
| DU145_23 | 20 | 5 | C | DU145 | 321.1689 | 97.3239 | 30.08193 | 139.7925 | 1235.129 | 32.73622 | 2099.542 | 16100.91 |
| PANC10_6 | 10 | 2 | A | PANC10 | 275.0814 | 24.77833 | 4.619688 | 152.4497 | 294.4001 | 27.71813 | 1201.959 | 24722.47 |
| LNCaP_24 | 10 | 2 | A | LNCaP | 191.2567 | 137.5476 | 7.859865 | 328.8044 | 331.4243 | 32.74944 | 1923.047 | 15162.99 |
| 22RV1_8 | 10 | 2 | A | 22RV1 | 210.2406 | 129.0285 | 4.553949 | 465.2618 | 245.1542 | 15.93882 | 881.9481 | 10532.52 |
| PC3_8 | 10 | 2 | A | PC3 | 1268.069 | 676.1431 | 25.66617 | 421.0856 | 741.9127 | 52.13441 | 1305.766 | 32049.02 |
| DU145_8 | 10 | 2 | A | DU145 | 484.3028 | 162.0973 | 97.45725 | 226.7373 | 1511.582 | 72.5957 | 3500.505 | 20059.29 |
| PANC10_17 | 10 | 5 | B | PANC10 | 322.1212 | 23.81882 | 3.402689 | 168.4331 | 326.6581 | 23.25171 | 1291.32 | 21974 |
| LNCaP_8 | 10 | 5 | B | LNCaP | 177.4626 | 216.7482 | 10.83741 | 356.2799 | 376.6001 | 40.64029 | 1574.134 | 14511.29 |
| 22RV1_17 | 10 | 5 | B | 22RV1 | 239.3598 | 120.3411 | 5.28972 | 435.0795 | 323.9953 | 15.86916 | 756.4299 | 10143.04 |
| PC3_17 | 10 | 5 | B | PC3 | 972.7494 | 524.2362 | 29.85234 | 401.9144 | 527.1486 | 72.81059 | 1108.905 | 25722.52 |
| DU145_17 | 10 | 5 | B | DU145 | 321.8763 | 162.586 | 56.02626 | 221.9079 | 1329.25 | 71.40601 | 2352.004 | 19793.75 |
| PANC10_26 | 20 | 5 | C | PANC10 | 210.1558 | 32.4252 | 2.634548 | 134.3619 | 362.9596 | 7.29567 | 992.0085 | 20848.8 |
| LNCaP_17 | 20 | 2 | C | LNCaP | 144.6707 | 201.7776 | 6.345206 | 418.7836 | 253.8082 | 31.72603 | 1463.205 | 10600.3 |
| LNCaP_18 | 20 | 5 | C | LNCaP | 200.3051 | 120.9572 | 0.967658 | 311.5857 | 244.8174 | 20.32081 | 1815.326 | 11864.45 |
| 22RV1_25 | 20 | 2 | C | 22RV1 | 208.1508 | 121.137 | 5.118463 | 380.4724 | 226.9185 | 23.88616 | 759.2387 | 9999.771 |
| 22RV1_26 | 20 | 5 | C | 22RV1 | 192.6104 | 118.7764 | 1.605087 | 393.2463 | 287.3105 | 16.05087 | 828.2248 | 10298.24 |
| PC3_21 | 20 | 5 | C | PC3 | 675.8017 | 388.9932 | 10.86022 | 359.8681 | 427.004 | 23.69502 | 776.9992 | 21501.26 |
| PC3_24 | 20 | 2 | C | PC3 | 710.7665 | 408.105 | 10.25144 | 335.8567 | 405.6641 | 20.01472 | 809.8638 | 21619.31 |
| DU145_25 | 20 | 2 | C | DU145 | 327.3233 | 93.92135 | 39.62393 | 168.0582 | 1223.67 | 43.63578 | 1988.066 | 17555.27 |
| DU145_26 | 20 | 5 | C | DU145 | 0 | 0 | 0 | 241.1813 | 482.3626 | 0 | 1688.269 | 6994.258 |
| PC3_PSI_4 | 20 | 0.5 | A | PC3 | 1470.105 | 299.8807 | 7.468294 | 589.4208 | 498.0778 | 88.47056 | 2114.676 | 20392.47 |
| PC3_PSI_7 | 20 | 2 | A | PC3 | 913.9944 | 249.5563 | 6.27251 | 402.3367 | 398.7524 | 49.28401 | 1836.053 | 21830.13 |
| PC3_PSI_10 | 20 | 5 | A | PC3 | 1082.442 | 231.3616 | 11.47627 | 386.0616 | 405.8008 | 42.23267 | 1590.611 | 23326.66 |
| PC3_PSI_5 | 20 | 0.5 | B | PC3 | 1393.246 | 294.2318 | 10.64655 | 573.4617 | 486.8376 | 80.33304 | 2149.635 | 19581.9 |
| PC3_PSI_8 | 20 | 2 | B | PC3 | 929.2346 | 237.8557 | 8.875211 | 387.8467 | 434.8853 | 56.80135 | 1786.58 | 20405 |
| PC3_PSI_11 | 20 | 5 | B | PC3 | 955.0823 | 239.8718 | 12.81452 | 364.4129 | 430.8883 | 49.25581 | 1538.143 | 23672.02 |
| PC3_PSI_6 | 20 | 0.5 | C | PC3 | 1275.758 | 348.6979 | 1.400393 | 639.9797 | 494.3388 | 113.4318 | 1960.55 | 20218.88 |
| PC3_PSI_9 | 20 | 2 | C | PC3 | 867.4324 | 285.8406 | 11.47535 | 366.6897 | 484.0513 | 45.90142 | 1520.484 | 22023.81 |
| PC3_PSI_12 | 20 | 5 | C | PC3 | 1123.57 | 261.2875 | 9.009913 | 433.8106 | 426.8029 | 52.05727 | 1841.359 | 19512.13 |
| PANC10_1 | 1 | 2 | A | PANC10 | 731.0598 | 187.9868 | 26.10928 | 313.3113 | 1096.59 | 219.3179 | 1921.643 | 20991.86 |
| LNCaP_19 | 1 | 2 | A | LNCaP | 702.2483 | 1298.139 | 193.7739 | 971.7834 | 1169.928 | 273.906 | 5826.33 | 49116.59 |
| 22RV1_1 | 1 | 2 | A | 22RV1 | 214.5837 | 163.0836 | 11.44447 | 504.9871 | 266.0838 | 58.65289 | 826.8627 | 10547.51 |
| PC3_1 | 1 | 2 | A | PC3 | 2573.914 | 1346.416 | 88.59052 | 662.4336 | 1702.375 | 122.9094 | 1581.86 | 31345.88 |
| DU145_1 | 1 | 2 | A | DU145 | 1051.173 | 659.8602 | 338.699 | 599.574 | 3639.096 | 358.429 | 6371.707 | 26577.46 |
| PANC10_10 | 1 | 5 | B | PANC10 | 804.5866 | 418.4743 | 147.303 | 571.3569 | 1063.483 | 446.3726 | 2510.846 | 31510.56 |
| LNCaP_3 | 1 | 5 | B | LNCaP | 604.2898 | 627.438 | 92.59279 | 855.265 | 741.9607 | 159.6007 | 4498.061 | 44259.36 |
| 22RV1_10 | 1 | 5 | B | 22RV1 | 580.0314 | 467.6222 | 151.7524 | 784.6161 | 753.1415 | 229.3147 | 2421.294 | 24631.1 |
| DU145_10 | 1 | 5 | B | DU145 | 1203.968 | 624.6599 | 214.7803 | 558.7712 | 2454.143 | 245.5855 | 6173.865 | 31692.51 |
| PANC10_2 | 1 | 2 | A | PANC10 | 1183.027 | 307.4562 | 149.4509 | 695.9279 | 1512.121 | 346.7059 | 5590.067 | 78193.51 |
| 22RV1_4 | 1 | 2 | A | 22RV1 | 532.976 | 494.5483 | 162.0648 | 826.1964 | 652.4362 | 311.5988 | 2659.868 | 25173.51 |
| PC3_4 | 1 | 2 | A | PC3 | 4049.774 | 3173.175 | 378.6605 | 1172.901 | 2545.545 | 256.5425 | 3821.631 | 65465.66 |
| DU145_4 | 1 | 2 | A | DU145 | 1195.26 | 788.3113 | 406.9489 | 642.1021 | 3689.346 | 383.7992 | 8898.048 | 36787.45 |
| PANC10_13 | 1 | 5 | B | PANC10 | 772.2797 | 374.3285 | 86.31362 | 516.9731 | 1433.715 | 536.9616 | 3733.291 | 68947.32 |
| LNCaP_6 | 1 | 5 | B | LNCaP | 526.9173 | 551.0878 | 56.80072 | 831.4659 | 807.2953 | 123.2696 | 4291.476 | 43798.19 |
| 22RV1_13 | 1 | 5 | B | 22RV1 | 526.6807 | 412.5491 | 108.8962 | 703.637 | 692.1191 | 216.7453 | 2371.634 | 24520.49 |
| PC3_13 | 1 | 5 | B | PC3 | 4524.925 | 3367.721 | 270.9948 | 1122.156 | 2841.377 | 296.029 | 4216.379 | 64623.19 |
| DU145_13 | 1 | 5 | B | DU145 | 1366.53 | 885.2882 | 312.0682 | 808.0923 | 2534.322 | 594.572 | 6794.874 | 49986.75 |
| PANC10_3 | 1 | 2 | A | PANC10 | 867.7696 | 348.604 | 80.79235 | 647.8349 | 1524.581 | 323.1694 | 3840.629 | 65821.82 |
| LNCaP_21 | 1 | 2 | A | LNCaP | 818.9263 | 785.0397 | 141.1942 | 1016.598 | 1157.792 | 200.4958 | 5475.511 | 50954.16 |
| 22RV1_7 | 1 | 2 | A | 22RV1 | 719.2968 | 771.1298 | 254.6183 | 940.2691 | 1096.678 | 405.5706 | 4000.236 | 34090.67 |
| PC3_7 | 1 | 2 | A | PC3 | 4536.24 | 3476.616 | 242.0746 | 1253.534 | 2829.208 | 184.5271 | 4467.433 | 75690.53 |
| DU145_7 | 1 | 2 | A | DU145 | 1027.857 | 677.8808 | 337.3639 | 457.1754 | 3470.329 | 259.5916 | 6680.016 | 32923.99 |
| PANC10_16 | 1 | 5 | B | PANC10 | 758.2926 | 364.7887 | 74.44668 | 503.0468 | 1655.907 | 450.9342 | 3356.482 | 61379.16 |
| LNCaP_9 | 1 | 5 | B | LNCaP | 570.9571 | 438.3332 | 47.20511 | 745.1664 | 690.0938 | 122.5085 | 4372.093 | 45783.34 |
| 22RV1_16 | 1 | 5 | B | 22RV1 | 550.355 | 384.2356 | 128.3036 | 709.0463 | 815.7409 | 237.0241 | 1979.927 | 29000.67 |
| PC3_16 | 1 | 5 | B | PC3 | 3915.276 | 2814.212 | 252.5061 | 1071.863 | 2282.289 | 302.3202 | 3744.648 | 70023.21 |
| DU145_16 | 1 | 5 | B | DU145 | 961.737 | 931.1199 | 90.05028 | 1010.364 | 1732.567 | 781.6364 | 3632.628 | 30377.56 |

TABLE 12
Oxygen-Sensitive Candidates

| | O2 | PSI | Expt | CellLine | P4HA1 | PDK1 | HK2 | SLC2A1 |
|---|---|---|---|---|---|---|---|---|
| LNCaP_25 | 20 | 0 | A | LNCaP | 649.5917 | 517.6434 | 827.2145 | 779.8484 |
| 22RV1_3 | 20 | 0 | A | 22RV1 | 630.8675 | 163.4094 | 1745.266 | 1078.234 |
| PC3_3 | 20 | 0 | A | PC3 | 2446.465 | 314.3372 | 1463.796 | 1660.33 |
| DU145_3 | 20 | 0 | A | DU145 | 970.0978 | 260.2968 | 407.9442 | 4884.393 |
| PANC10_12 | 20 | 0 | B | PANC10 | 380.7663 | 162.59 | 1438.296 | 993.6055 |
| LNCaP_1 | 20 | 0 | B | LNCaP | 743.5561 | 615.2785 | 860.4817 | 648.1993 |
| 22RV1_12 | 20 | 0 | B | 22RV1 | 691.4687 | 140.7887 | 1638.674 | 762.7541 |
| DU145_12 | 20 | 0 | B | DU145 | 1446.568 | 546.0028 | 391.9439 | 3900.182 |
| PANC10_21 | 20 | 0 | C | PANC10 | 647.8829 | 346.2442 | 1475.071 | 673.9396 |
| LNCaP_10 | 20 | 0 | C | LNCaP | 632.5891 | 510.6683 | 658.5723 | 1055.315 |
| 22RV1_21 | 20 | 0 | C | 22RV1 | 615.9897 | 192.7907 | 1689.269 | 945.1445 |
| PC3_25 | 20 | 0 | C | PC3 | 1517.876 | 222.2692 | 1004.756 | 1670.738 |
| DU145_21 | 20 | 0 | C | DU145 | 1021.792 | 319.0576 | 492.7219 | 3892.301 |
| PANC10_8 | 20 | 0 | A | PANC10 | 610.8968 | 233.3287 | 1392.699 | 760.5908 |
| LNCaP_26 | 20 | 0 | A | LNCaP | 806.4939 | 585.7116 | 718.9108 | 689.7165 |
| 22RV1_6 | 20 | 0 | A | 22RV1 | 688.0363 | 169.4418 | 1500.33 | 909.8509 |
| PC3_6 | 20 | 0 | A | PC3 | 2284.137 | 313.7671 | 1392.57 | 1420.363 |
| DU145_6 | 20 | 0 | A | DU145 | 1002.637 | 309.6547 | 439.7327 | 4949.871 |
| PANC10_15 | 20 | 0 | B | PANC10 | 557.7053 | 237.8784 | 1526.291 | 630.5484 |
| LNCaP_4 | 20 | 0 | B | LNCaP | 676.7228 | 544.6136 | 812.8762 | 1005.648 |
| 22RV1_15 | 20 | 0 | B | 22RV1 | 695.9886 | 139.3927 | 1692.208 | 884.12 |
| PC3_15 | 20 | 0 | B | PC3 | 3292.687 | 381.8236 | 1419.364 | 2126.063 |
| DU145_15 | 20 | 0 | B | DU145 | 1396.275 | 404.2644 | 595.8111 | 4661.642 |
| PANC10_24 | 20 | 0 | C | PANC10 | 442.4396 | 237.8169 | 1573.321 | 888.9716 |
| LNCaP_13 | 20 | 0 | C | LNCaP | 701.3473 | 571.904 | 890.8053 | 1093.208 |
| 22RV1_24 | 20 | 0 | C | 22RV1 | 732.7024 | 187.3962 | 1635.074 | 869.451 |
| PC3_26 | 20 | 0 | C | PC3 | 1839.411 | 267.9427 | 989.3862 | 2629.381 |
| DU145_24 | 20 | 0 | C | DU145 | 889.4556 | 210.6179 | 708.0002 | 3149.548 |
| PANC10_9 | 20 | 0 | A | PANC10 | 454.5376 | 347.3966 | 1451.274 | 811.6744 |
| LNCaP_27 | 20 | 0 | A | LNCaP | 683.3315 | 522.0734 | 905.0615 | 776.055 |
| 22RV1_9 | 20 | 0 | A | 22RV1 | 734.8624 | 125.214 | 1239.824 | 927.8151 |
| PC3_9 | 20 | 0 | A | PC3 | 2725.102 | 467.9921 | 1644.13 | 1780.161 |
| DU145_9 | 20 | 0 | A | DU145 | 1434.512 | 503.8392 | 614.9478 | 4513.651 |
| PANC10_18 | 20 | 0 | B | PANC10 | 734.739 | 325.4549 | 1568.101 | 761.0384 |
| LNCaP_7 | 20 | 0 | B | LNCaP | 860.5564 | 654.5666 | 808.2747 | 734.0347 |
| 22RV1_18 | 20 | 0 | B | 22RV1 | 647.1424 | 179.7618 | 1580.62 | 837.1763 |
| PC3_18 | 20 | 0 | B | PC3 | 2106.253 | 341.0059 | 1355.905 | 2117.755 |
| DU145_18 | 20 | 0 | B | DU145 | 1187.261 | 348.1306 | 602.2207 | 3117.803 |
| PANC10_27 | 20 | 0 | C | PANC10 | 454.3215 | 276.0217 | 1273.815 | 1016.651 |
| LNCaP_16 | 20 | 0 | C | LNCaP | 706.6135 | 599.8143 | 702.8879 | 1046.881 |
| 22RV1_27 | 20 | 0 | C | 22RV1 | 679.6124 | 180.2847 | 1556.728 | 803.1783 |
| PC3_27 | 20 | 0 | C | PC3 | 1478.965 | 232.9337 | 1005.224 | 1622.662 |
| DU145_27 | 20 | 0 | C | DU145 | 332.0628 | 110.6876 | 553.4381 | 2324.44 |
| PC3_PSI_1 | 20 | 0 | A | PC3 | 5714.151 | 404.2344 | 1986.444 | 2360.812 |
| PC3_PSI_2 | 20 | 0 | B | PC3 | 6109.165 | 376.4844 | 1645.887 | 2259.563 |
| PC3_PSI_3 | 20 | 0 | C | PC3 | 5512.204 | 439.3843 | 2096.627 | 2465.965 |
| PANC10_4 | 10 | 2 | A | PANC10 | 465.5349 | 346.3125 | 2496.856 | 1017.364 |
| PC3_2 | 10 | 2 | A | PC3 | 3003.416 | 527.9191 | 1531.199 | 1958.673 |
| DU145_2 | 10 | 2 | A | DU145 | 653.6147 | 435.3739 | 301.3275 | 5786.152 |
| PANC10_11 | 10 | 5 | B | PANC10 | 370.9092 | 186.3727 | 1402.845 | 747.3271 |
| LNCaP_2 | 10 | 5 | B | LNCaP | 938.4397 | 1060.845 | 896.231 | 1066.473 |
| 22RV1_11 | 10 | 5 | B | 22RV1 | 587.6074 | 191.8134 | 1524.487 | 815.2069 |
| DU145_11 | 10 | 5 | B | DU145 | 1067.259 | 684.08 | 218.6235 | 4109.182 |
| PANC10_19 | 20 | 2 | C | PANC10 | 358.9844 | 322.9525 | 1034.249 | 899.4629 |
| PANC10_20 | 20 | 5 | C | PANC10 | 426.7352 | 347.4141 | 1170.755 | 843.0173 |
| LNCaP_11 | 20 | 2 | C | LNCaP | 641.7725 | 672.8978 | 542.4682 | 1050.847 |
| LNCaP_12 | 20 | 5 | C | LNCaP | 646.2691 | 630.5914 | 674.1405 | 921.4995 |
| 22RV1_19 | 20 | 2 | C | 22RV1 | 481.8356 | 168.3212 | 1372.268 | 898.1415 |
| PC3_19 | 20 | 5 | C | PC3 | 1454.841 | 293.3005 | 1160.127 | 1879.95 |
| PC3_22 | 20 | 2 | C | PC3 | 1389.986 | 332.6084 | 1096.657 | 2104.619 |
| DU145_19 | 20 | 2 | C | DU145 | 771.0463 | 292.6448 | 372.5513 | 4544.296 |
| DU145_20 | 20 | 5 | C | DU145 | 777.2319 | 282.8096 | 354.0064 | 3331.418 |
| LNCaP_23 | 10 | 2 | A | LNCaP | 562.3244 | 757.2405 | 726.1919 | 867.6354 |
| 22RV1_5 | 10 | 2 | A | 22RV1 | 526.2672 | 200.8586 | 1475.302 | 942.0182 |
| PC3_5 | 10 | 2 | A | PC3 | 2330.702 | 503.894 | 1410.749 | 1630.336 |
| DU145_5 | 10 | 2 | A | DU145 | 895.9417 | 565.7004 | 529.7829 | 4733.126 |
| PANC10_14 | 10 | 5 | B | PANC10 | 469.8131 | 350.4688 | 1268.243 | 837.9314 |
| LNCaP_5 | 10 | 5 | B | LNCaP | 1092.243 | 1116.671 | 734.5599 | 789.521 |
| 22RV1_14 | 10 | 5 | B | 22RV1 | 574.9599 | 165.7388 | 1411.343 | 768.8914 |
| PC3_14 | 10 | 5 | B | PC3 | 3483.102 | 719.0319 | 1566.307 | 2744.149 |
| DU145_14 | 10 | 5 | B | DU145 | 1287.727 | 807.8211 | 539.7442 | 4438.828 |
| PANC10_22 | 20 | 2 | C | PANC10 | 305.3755 | 231.9067 | 1287.936 | 823.788 |
| PANC10_23 | 20 | 5 | C | PANC10 | 376.0974 | 271.2032 | 1108.183 | 748.3903 |
| LNCaP_14 | 20 | 2 | C | LNCaP | 678.4435 | 713.4805 | 748.5175 | 1030.937 |
| LNCaP_15 | 20 | 5 | C | LNCaP | 724.0804 | 889.6881 | 620.1223 | 956.173 |
| 22RV1_22 | 20 | 2 | C | 22RV1 | 605.6675 | 225.7146 | 1423.256 | 872.7631 |
| 22RV1_23 | 20 | 5 | C | 22RV1 | 576.902 | 196.5914 | 1425.287 | 903.3841 |
| PC3_20 | 20 | 5 | C | PC3 | 2397.013 | 392.7347 | 1262.742 | 2501.521 |
| PC3_23 | 20 | 2 | C | PC3 | 2171.906 | 408.3301 | 1249.383 | 2355.338 |
| DU145_22 | 20 | 2 | C | DU145 | 704.0651 | 276.6811 | 521.5733 | 3496.778 |
| DU145_23 | 20 | 5 | C | DU145 | 755.5873 | 307.8974 | 391.9499 | 3486.85 |
| PANC10_6 | 10 | 2 | A | PANC10 | 496.4065 | 395.1933 | 1267.474 | 753.0091 |
| LNCaP_24 | 10 | 2 | A | LNCaP | 818.736 | 872.445 | 791.2264 | 755.857 |
| 22RV1_8 | 10 | 2 | A | 22RV1 | 557.8587 | 231.4924 | 1137.728 | 997.3148 |
| PC3_8 | 10 | 2 | A | PC3 | 3033.42 | 677.7473 | 1817.486 | 2119.063 |
| DU145_8 | 10 | 2 | A | DU145 | 782.6413 | 521.0979 | 348.0616 | 3922.157 |
| PANC10_17 | 10 | 5 | B | PANC10 | 537.6248 | 352.7454 | 1374.119 | 722.5042 |
| LNCaP_8 | 10 | 5 | B | LNCaP | 732.88 | 872.4116 | 803.3231 | 1007.879 |
| 22RV1_17 | 10 | 5 | B | 22RV1 | 677.0841 | 207.6215 | 1496.991 | 763.0421 |
| PC3_17 | 10 | 5 | B | PC3 | 2249.119 | 491.4714 | 1362.286 | 2311.736 |
| DU145_17 | 10 | 5 | B | DU145 | 949.1507 | 449.3086 | 342.7489 | 3878.994 |
| PANC10_26 | 20 | 5 | C | PANC10 | 395.9928 | 313.5112 | 1105.091 | 765.0321 |
| LNCaP_17 | 20 | 2 | C | LNCaP | 540.6116 | 719.5464 | 697.9727 | 1271.579 |
| LNCaP_18 | 20 | 5 | C | LNCaP | 748.967 | 979.2695 | 737.3551 | 769.2878 |
| 22RV1_25 | 20 | 2 | C | 22RV1 | 544.2632 | 220.0939 | 1337.625 | 897.4372 |
| 22RV1_26 | 20 | 5 | C | 22RV1 | 598.6974 | 218.2918 | 1362.719 | 882.7977 |
| PC3_21 | 20 | 5 | C | PC3 | 1519.443 | 293.2259 | 1094.414 | 1690.245 |
| PC3_24 | 20 | 2 | C | PC3 | 1659.757 | 289.4812 | 1111.061 | 1648.041 |
| DU145_25 | 20 | 2 | C | DU145 | 677.9484 | 283.5227 | 335.7317 | 3924.363 |
| DU145_26 | 20 | 5 | C | DU145 | 723.5439 | 0 | 1447.088 | 2411.813 |
| PC3_PSI_4 | 20 | 0.5 | A | PC3 | 5684.521 | 567.5904 | 2330.108 | 2964.338 |
| PC3_PSI_7 | 20 | 2 | A | PC3 | 3168.962 | 676.5351 | 2363.84 | 1947.166 |
| PC3_PSI_10 | 20 | 5 | A | PC3 | 2911.759 | 659.1968 | 2217.215 | 2038.185 |
| PC3_PSI_5 | 20 | 0.5 | B | PC3 | 5978.036 | 587.9798 | 2240.13 | 2297.235 |
| PC3_PSI_8 | 20 | 2 | B | PC3 | 2980.296 | 670.966 | 2292.467 | 1868.232 |
| PC3_PSI_11 | 20 | 5 | B | PC3 | 2440.766 | 653.941 | 2114.396 | 1842.888 |
| PC3_PSI_6 | 20 | 0.5 | C | PC3 | 5698.2 | 504.1415 | 2181.813 | 3509.385 |
| PC3_PSI_9 | 20 | 2 | C | PC3 | 2633.072 | 619.6691 | 2340.451 | 2198.052 |
| PC3_PSI_12 | 20 | 5 | C | PC3 | 3495.846 | 623.3525 | 2334.235 | 2044.583 |
| PANC10_1 | 1 | 2 | A | PANC10 | 1154.03 | 1091.368 | 4140.931 | 1968.64 |
| LNCaP_19 | 1 | 2 | A | LNCaP | 2533.63 | 3489.387 | 2233.499 | 2788.596 |
| 22RV1_1 | 1 | 2 | A | 22RV1 | 570.7928 | 193.1254 | 1821.101 | 1194.516 |
| PC3_1 | 1 | 2 | A | PC3 | 5150.222 | 1217.122 | 2708.795 | 3858.077 |
| DU145_1 | 1 | 2 | A | DU145 | 1531.27 | 959.0991 | 1274.78 | 8591.336 |
| PANC10_10 | 1 | 5 | B | PANC10 | 1489.769 | 1259.887 | 3536.387 | 2274.268 |
| LNCaP_3 | 1 | 5 | B | LNCaP | 2708.339 | 2442.744 | 1751.953 | 1772.665 |
| 22RV1_10 | 1 | 5 | B | 22RV1 | 1570.356 | 460.8777 | 3189.049 | 1848.007 |
| DU145_10 | 1 | 5 | B | DU145 | 1849.165 | 1211.669 | 1052.509 | 7289.696 |
| PANC10_2 | 1 | 2 | A | PANC10 | 3117.334 | 2465.185 | 3551.597 | 2408.323 |
| 22RV1_4 | 1 | 2 | A | 22RV1 | 1187.918 | 595.63 | 2880.41 | 1901.338 |
| PC3_4 | 1 | 2 | A | PC3 | 7777.686 | 2626.01 | 3321.799 | 6459.001 |
| DU145_4 | 1 | 2 | A | DU145 | 1791.063 | 1147.742 | 1363.401 | 8994.303 |
| PANC10_13 | 1 | 5 | B | PANC10 | 2011.562 | 1618.153 | 2430.41 | 2528.535 |
| LNCaP_6 | 1 | 5 | B | LNCaP | 2837.619 | 2337.289 | 1760.822 | 1827.291 |
| 22RV1_13 | 1 | 5 | B | 22RV1 | 1331.884 | 471.1855 | 2938.103 | 1777.94 |
| PC3_13 | 1 | 5 | B | PC3 | 8870.855 | 2186.108 | 3833.356 | 8164.266 |
| DU145_13 | 1 | 5 | B | DU145 | 2156.555 | 1241.703 | 998.6182 | 8236.958 |
| PANC10_3 | 1 | 2 | A | PANC10 | 2281.636 | 1934.528 | 2818.755 | 2720.009 |
| LNCaP_21 | 1 | 2 | A | LNCaP | 3128.863 | 3035.675 | 2180.038 | 1843.996 |
| 22RV1_7 | 1 | 2 | A | 22RV1 | 1699.577 | 959.3655 | 3535.557 | 2450.701 |
| PC3_7 | 1 | 2 | A | PC3 | 8564.561 | 2882.376 | 3866.938 | 7705.728 |
| DU145_7 | 1 | 2 | A | DU145 | 2227.022 | 982.6645 | 873.3627 | 7695.261 |
| PANC10_16 | 1 | 5 | B | PANC10 | 2214.257 | 1636.763 | 2495.027 | 2823.656 |
| LNCaP_9 | 1 | 5 | B | LNCaP | 2806.456 | 2419.824 | 1773.563 | 1452.119 |
| 22RV1_16 | 1 | 5 | B | 22RV1 | 1160.135 | 524.6943 | 2679.52 | 1509.931 |
| PC3_16 | 1 | 5 | B | PC3 | 7363.903 | 1915.84 | 3168.637 | 7527.66 |
| DU145_16 | 1 | 5 | B | DU145 | 3322.855 | 633.954 | 520.4906 | 5664.163 |

| | TCAF2 | PGK1 | NDRG1 | RP11.61L23.2 |
|---|---|---|---|---|
| LNCaP_25 | 5.074935 | 6913.754 | 590.3842 | 1.691645 |
| 22RV1_3 | 13.39421 | 5498.325 | 375.038 | 17.41248 |
| PC3_3 | 48.40442 | 11756.44 | 1036.321 | 15.74602 |
| DU145_3 | 5.468421 | 6583.978 | 928.5378 | 5.468421 |
| PANC10_12 | 8.337949 | 8091.979 | 3051.689 | 15.28624 |
| LNCaP_1 | 13.6224 | 6934.938 | 532.4089 | 1.1352 |
| 22RV1_12 | 9.801746 | 5673.429 | 393.852 | 18.71243 |
| DU145_12 | 4.531143 | 12527.48 | 1383.132 | 3.398358 |
| PANC10_21 | 24.73173 | 8389.797 | 2002.828 | 15.89897 |
| LNCaP_10 | 9.993509 | 8129.72 | 811.473 | 7.994808 |
| 22RV1_21 | 24.68661 | 5088.968 | 517.2432 | 17.63329 |
| PC3_25 | 45.44538 | 10514.41 | 714.732 | 23.96211 |
| DU145_21 | 7.067732 | 6815.313 | 1210.601 | 6.058056 |
| PANC10_8 | 12.12097 | 8984.668 | 2512.071 | 23.63589 |
| LNCaP_26 | 27.3697 | 7249.322 | 607.6074 | 0 |
| 22RV1_6 | 9.242278 | 5890.412 | 443.6294 | 18.48456 |
| PC3_6 | 46.07768 | 12791.31 | 1051.741 | 32.91263 |
| DU145_6 | 3.453398 | 7278.613 | 935.871 | 13.81359 |
| PANC10_15 | 13.65809 | 7513.087 | 1914.409 | 25.03983 |
| LNCaP_4 | 9.436374 | 8479.256 | 825.0087 | 4.04416 |
| 22RV1_15 | 11.69729 | 5622.496 | 439.6231 | 6.823418 |
| PC3_15 | 75.93084 | 14487.6 | 1961.185 | 35.2536 |
| DU145_15 | 9.073265 | 9858.607 | 2695.768 | 7.056984 |
| PANC10_24 | 27.28301 | 7942.54 | 1993.479 | 25.46415 |
| LNCaP_13 | 10.59082 | 8366.744 | 839.0279 | 4.707029 |
| 22RV1_24 | 24.47969 | 5327.287 | 538.5532 | 15.19429 |
| PC3_26 | 50.04677 | 11911.9 | 878.5134 | 23.09851 |
| DU145_24 | 16.20138 | 5913.503 | 959.1215 | 1.620138 |
| PANC10_9 | 45.45376 | 8035.576 | 1733.736 | 6.493395 |
| LNCaP_27 | 16.12582 | 7077.218 | 713.5674 | 6.047182 |
| 22RV1_9 | 10.26344 | 5603.839 | 504.9613 | 16.42151 |
| PC3_9 | 82.85027 | 13963.07 | 1870.289 | 48.70252 |
| DU145_9 | 14.30111 | 9117.509 | 1654.529 | 5.500428 |
| PANC10_18 | 24.65567 | 8032.818 | 2118.744 | 31.23052 |
| LNCaP_7 | 7.319434 | 7679.132 | 725.6696 | 2.091267 |
| 22RV1_18 | 20.5442 | 5183.559 | 493.0609 | 11.55611 |
| PC3_18 | 74.4259 | 13456.2 | 2266.607 | 43.97894 |
| DU145_18 | 12.65929 | 7606.427 | 939.5005 | 4.521177 |
| PANC10_27 | 15.42979 | 8119.496 | 2350.471 | 24.00189 |
| LNCaP_16 | 14.90222 | 7503.267 | 909.0353 | 1.241851 |
| 22RV1_27 | 31.39789 | 5252.563 | 513.5074 | 17.2182 |
| PC3_27 | 38.71292 | 10959.04 | 728.3278 | 22.30914 |
| DU145_27 | 0 | 7858.821 | 1106.876 | 0 |
| PC3_PSI_1 | 136.1339 | 11451.22 | 8597.273 | 67.37239 |
| PC3_PSI_2 | 101.8413 | 12176.28 | 8531.675 | 62.41887 |
| PC3_PSI_3 | 99.49826 | 10847.7 | 8207.413 | 55.71903 |
| PANC10_4 | 20.43812 | 6899 | 9683.125 | 10.21906 |
| PC3_2 | 74.74961 | 13316.53 | 1232.785 | 29.19907 |
| DU145_2 | 22.15643 | 7367.014 | 1418.012 | 5.539108 |
| PANC10_11 | 27.54277 | 7995.665 | 2921.369 | 19.27994 |
| LNCaP_2 | 28.13912 | 11285.19 | 1315.504 | 5.627824 |
| 22RV1_11 | 5.725772 | 5800.923 | 302.7502 | 15.03015 |
| DU145_11 | 7.052371 | 13331.33 | 1395.194 | 4.701581 |
| PANC10_19 | 21.35223 | 8454.15 | 2243.319 | 16.01418 |
| PANC10_20 | 14.75741 | 8604.187 | 2191.476 | 24.59569 |
| LNCaP_11 | 25.19661 | 8225.953 | 867.0599 | 13.33938 |
| LNCaP_12 | 10.45179 | 8051.363 | 918.0156 | 5.225895 |
| 22RV1_19 | 20.55832 | 5171.702 | 395.7476 | 25.6979 |
| PC3_19 | 50.17912 | 10696.63 | 830.4291 | 22.26257 |
| PC3_22 | 43.0807 | 11520.29 | 807.7632 | 21.54035 |
| DU145_19 | 7.264232 | 6830.453 | 906.9912 | 0 |
| DU145_20 | 13.84383 | 6998.054 | 810.8527 | 4.944224 |
| LNCaP_23 | 12.07445 | 7374.039 | 721.0171 | 5.174764 |
| 22RV1_5 | 11.40246 | 5530.191 | 318.3916 | 7.016895 |
| PC3_5 | 63.17937 | 13791.59 | 1123.36 | 30.04872 |
| DU145_5 | 22.94728 | 6909.127 | 2174.005 | 21.94957 |
| PANC10_14 | 26.05403 | 8333.929 | 2506.23 | 17.64951 |
| LNCaP_5 | 10.46879 | 8439.587 | 819.1825 | 2.617197 |
| 22RV1_14 | 12.81486 | 5604.364 | 309.2652 | 11.96053 |
| PC3_14 | 132.6007 | 19719.53 | 3896.468 | 67.85669 |
| DU145_14 | 21.5419 | 12404.54 | 3389.258 | 13.16449 |
| PANC10_22 | 21.661 | 7066.846 | 1907.062 | 21.88431 |
| PANC10_23 | 12.50035 | 7958.916 | 1831.572 | 10.32637 |
| LNCaP_14 | 9.555543 | 8537.347 | 745.3323 | 2.123454 |
| LNCaP_15 | 8.461708 | 9301.835 | 704.7394 | 3.626446 |
| 22RV1_22 | 13.79367 | 5359.467 | 392.4926 | 21.31749 |
| 22RV1_23 | 14.04224 | 5195.629 | 359.2473 | 12.87205 |
| PC3_20 | 81.87521 | 13993.34 | 1294.028 | 43.26739 |
| PC3_23 | 81.47088 | 13702.72 | 1202.549 | 34.63732 |
| DU145_22 | 12.95103 | 5573.652 | 898.3305 | 1.177366 |
| DU145_23 | 6.193339 | 5824.393 | 844.9484 | 1.769525 |
| PANC10_6 | 20.99858 | 7886.647 | 1809.238 | 29.39801 |
| LNCaP_24 | 7.859865 | 7931.914 | 858.0353 | 11.7898 |
| 22RV1_8 | 13.66185 | 6635.862 | 321.8124 | 9.107898 |
| PC3_8 | 135.5495 | 15911.42 | 2852.153 | 60.95715 |
| DU145_8 | 13.92246 | 7322.221 | 1228.16 | 6.961232 |
| PANC10_17 | 10.20807 | 7970.231 | 1772.234 | 30.6242 |
| LNCaP_8 | 21.67482 | 8740.373 | 957.7563 | 4.064029 |
| 22RV1_17 | 14.54673 | 5682.482 | 416.5654 | 10.57944 |
| PC3_17 | 117.9531 | 15416.19 | 2191.599 | 45.87067 |
| DU145_17 | 12.08409 | 7421.831 | 1130.412 | 5.49277 |
| PANC10_26 | 7.498328 | 8108.327 | 1471.699 | 14.794 |
| LNCaP_17 | 10.15233 | 7925.162 | 719.5464 | 3.807124 |
| LNCaP_18 | 13.54721 | 7599.983 | 600.9154 | 6.773603 |
| 22RV1_25 | 22.18001 | 5497.229 | 406.0647 | 15.35539 |
| 22RV1_26 | 20.86613 | 5348.149 | 378.8005 | 16.05087 |
| PC3_21 | 47.88369 | 11973.88 | 763.1771 | 24.18867 |
| PC3_24 | 60.04415 | 12247.05 | 845.4998 | 28.80167 |
| DU145_25 | 7.364225 | 6414.625 | 902.9969 | 3.792026 |
| DU145_26 | 0 | 4582.445 | 1205.906 | 0 |
| PC3_PSI_4 | 116.0458 | 10641.75 | 6466.968 | 55.15048 |
| PC3_PSI_7 | 65.41332 | 11098.76 | 5544.003 | 27.77826 |
| PC3_PSI_10 | 69.31666 | 12269.51 | 4685.072 | 45.90507 |
| PC3_PSI_5 | 109.853 | 10928.2 | 7134.154 | 59.52388 |
| PC3_PSI_8 | 65.67656 | 11224.48 | 5054.433 | 49.70118 |
| PC3_PSI_11 | 75.28531 | 11772.94 | 4058.599 | 29.63358 |
| PC3_PSI_6 | 138.6389 | 11600.86 | 6114.117 | 60.21691 |
| PC3_PSI_9 | 72.50337 | 10328.86 | 5277.098 | 31.81803 |
| PC3_PSI_12 | 64.40419 | 10479.2 | 6131.746 | 27.02974 |
| PANC10_1 | 104.4371 | 9649.989 | 16746.49 | 36.55299 |
| LNCaP_19 | 167.5489 | 25812.72 | 4163.954 | 37.88061 |
| 22RV1_1 | 14.30558 | 5440.413 | 343.334 | 17.1667 |
| PC3_1 | 228.2603 | 19178.65 | 3507.706 | 102.1584 |
| DU145_1 | 134.8219 | 11298.74 | 4204.691 | 44.94065 |
| PANC10_10 | 249.9687 | 14071.9 | 14980.26 | 214.2588 |
| LNCaP_3 | 65.78962 | 19872.12 | 3352.834 | 45.07807 |
| 22RV1_10 | 24.73002 | 10677.75 | 1065.639 | 31.47457 |
| DU145_10 | 103.5395 | 15795.34 | 4245.976 | 51.34191 |
| PANC10_2 | 311.985 | 30121.65 | 32468.58 | 255.6264 |
| 22RV1_4 | 37.59235 | 12027.88 | 614.0085 | 33.41543 |
| PC3_4 | 642.7761 | 54808.26 | 21785.28 | 307.6616 |
| DU145_4 | 197.3824 | 18925.56 | 10973 | 73.1046 |
| PANC10_13 | 191.7071 | 29308.47 | 24094.22 | 162.633 |
| LNCaP_6 | 65.2604 | 20293.57 | 3592.948 | 26.58757 |
| 22RV1_13 | 34.5536 | 9575.536 | 962.2655 | 33.50652 |
| PC3_13 | 699.7048 | 56612.26 | 37270.24 | 392.4105 |
| DU145_13 | 93.62045 | 27383.16 | 14038.14 | 70.62596 |
| PANC10_3 | 204.9732 | 30376.43 | 24958.85 | 187.0193 |
| LNCaP_21 | 163.7853 | 26070.1 | 3826.363 | 14.11942 |
| 22RV1_7 | 47.28626 | 16204.64 | 1003.924 | 42.7395 |
| PC3_7 | 636.1495 | 71921.8 | 46350.09 | 299.622 |
| DU145_7 | 91.43509 | 15639.6 | 9443.038 | 52.5489 |
| PANC10_16 | 173.3544 | 26166.94 | 30138.14 | 189.3073 |
| LNCaP_9 | 73.05553 | 19577.76 | 3049.225 | 32.59401 |
| 22RV1_16 | 23.63488 | 11998.41 | 1079.101 | 41.19222 |
| PC3_16 | 550.2457 | 61562.82 | 36378.06 | 275.4092 |
| DU145_16 | 34.21911 | 17995.65 | 11369.75 | 36.02011 |

TABLE 13
Oxygen-Sensitive Candidates

| | O2 | PSI | Expt | CellLine | BNIP3 | PPP1R3G | KDM3A | BHLHE40 |
|---|---|---|---|---|---|---|---|---|
| LNCaP_25 | 20 | 0 | A | LNCaP | 1884.493 | 0 | 336.6374 | 201.3058 |
| 22RV1_3 | 20 | 0 | A | 22RV1 | 377.7168 | 1.339421 | 310.7458 | 2.678843 |
| PC3_3 | 20 | 0 | A | PC3 | 535.9478 | 26.82655 | 506.7885 | 2151.956 |
| DU145_3 | 20 | 0 | A | DU145 | 1537.72 | 43.74737 | 534.8115 | 313.8873 |
| PANC10_12 | 20 | 0 | B | PANC10 | 6.94829 | 13.89658 | 821.2879 | 187.6038 |
| LNCaP_1 | 20 | 0 | B | LNCaP | 2310.132 | 1.1352 | 353.0473 | 188.4432 |
| 22RV1_12 | 20 | 0 | B | 22RV1 | 455.3357 | 1.782136 | 336.8237 | 5.346407 |
| DU145_12 | 20 | 0 | B | DU145 | 2355.062 | 58.90487 | 412.3341 | 404.4046 |
| PANC10_21 | 20 | 0 | C | PANC10 | 4.41638 | 14.57405 | 1080.247 | 197.4122 |
| LNCaP_10 | 20 | 0 | C | LNCaP | 2106.632 | 0 | 274.8215 | 273.8222 |
| 22RV1_21 | 20 | 0 | C | 22RV1 | 384.4058 | 2.351106 | 355.0169 | 8.22887 |
| PC3_25 | 20 | 0 | C | PC3 | 473.4583 | 21.48327 | 481.7211 | 1764.933 |
| DU145_21 | 20 | 0 | C | DU145 | 1772.991 | 56.54185 | 465.4606 | 379.6382 |
| PANC10_8 | 20 | 0 | A | PANC10 | 12.12097 | 7.272581 | 898.7699 | 173.3299 |
| LNCaP_26 | 20 | 0 | A | LNCaP | 1737.064 | 0 | 346.6829 | 209.8344 |
| 22RV1_6 | 20 | 0 | A | 22RV1 | 375.8526 | 0 | 370.718 | 1.02692 |
| PC3_6 | 20 | 0 | A | PC3 | 652.4015 | 31.44985 | 544.1555 | 1584.195 |
| DU145_6 | 20 | 0 | A | DU145 | 1697.921 | 55.25438 | 534.1256 | 370.6648 |
| PANC10_15 | 20 | 0 | B | PANC10 | 11.38174 | 3.414522 | 1059.64 | 175.2788 |
| LNCaP_4 | 20 | 0 | B | LNCaP | 2423.8 | 0 | 315.4445 | 227.821 |
| 22RV1_15 | 20 | 0 | B | 22RV1 | 447.4213 | 0.974774 | 380.1618 | 5.848644 |
| PC3_15 | 20 | 0 | B | PC3 | 859.6456 | 43.93141 | 709.411 | 1654.75 |
| DU145_15 | 20 | 0 | B | DU145 | 2327.797 | 101.8222 | 502.054 | 634.1204 |
| PANC10_24 | 20 | 0 | C | PANC10 | 9.549055 | 6.820754 | 841.681 | 201.4396 |
| LNCaP_13 | 20 | 0 | C | LNCaP | 2220.541 | 0 | 262.4169 | 267.1239 |
| 22RV1_24 | 20 | 0 | C | 22RV1 | 497.1909 | 4.220636 | 406.8693 | 11.81778 |
| PC3_26 | 20 | 0 | C | PC3 | 486.6086 | 29.25811 | 480.449 | 1790.905 |
| DU145_24 | 20 | 0 | C | DU145 | 1195.662 | 46.98399 | 460.1191 | 272.1831 |
| PANC10_9 | 20 | 0 | A | PANC10 | 9.740092 | 6.493395 | 986.996 | 181.8151 |
| LNCaP_27 | 20 | 0 | A | LNCaP | 1826.249 | 0 | 368.8781 | 288.249 |
| 22RV1_9 | 20 | 0 | A | 22RV1 | 418.7484 | 2.052688 | 336.6409 | 2.052688 |
| PC3_9 | 20 | 0 | A | PC3 | 882.2434 | 50.94172 | 802.1921 | 2231.359 |
| DU145_9 | 20 | 0 | A | DU145 | 3016.435 | 74.80582 | 741.4577 | 861.367 |
| PANC10_18 | 20 | 0 | B | PANC10 | 8.218557 | 8.218557 | 1145.667 | 261.3501 |
| LNCaP_7 | 20 | 0 | B | LNCaP | 2469.786 | 2.091267 | 397.3407 | 248.8608 |
| 22RV1_18 | 20 | 0 | B | 22RV1 | 360.8076 | 0 | 382.6358 | 1.284013 |
| PC3_18 | 20 | 0 | B | PC3 | 726.6674 | 42.62574 | 606.9094 | 1504.756 |
| DU145_18 | 20 | 0 | B | DU145 | 1682.782 | 56.06259 | 432.2245 | 348.1306 |
| PANC10_27 | 20 | 0 | C | PANC10 | 3.428841 | 15.42979 | 917.215 | 231.4468 |
| LNCaP_16 | 20 | 0 | C | LNCaP | 2055.264 | 0 | 289.3514 | 295.5606 |
| 22RV1_27 | 20 | 0 | C | 22RV1 | 369.6848 | 3.038506 | 383.8645 | 10.12835 |
| PC3_27 | 20 | 0 | C | PC3 | 432.4036 | 14.43532 | 459.9619 | 1728.958 |
| DU145_27 | 20 | 0 | C | DU145 | 996.1885 | 0 | 0 | 110.6876 |
| PC3_PSI_1 | 20 | 0 | A | PC3 | 1212.009 | 49.31381 | 1193.255 | 2956.051 |
| PC3_PSI_2 | 20 | 0 | B | PC3 | 1408.695 | 53.87734 | 1015.128 | 3076.922 |
| PC3_PSI_3 | 20 | 0 | C | PC3 | 1089.705 | 60.49494 | 1467.798 | 3081.262 |
| PANC10_4 | 10 | 2 | A | PANC10 | 6.812705 | 44.28258 | 1556.703 | 436.0131 |
| PC3_2 | 10 | 2 | A | PC3 | 756.8398 | 47.30249 | 706.0334 | 1985.537 |
| DU145_2 | 10 | 2 | A | DU145 | 1657.301 | 74.22405 | 546.156 | 310.19 |
| PANC10_11 | 10 | 5 | B | PANC10 | 7.344738 | 8.26283 | 893.3037 | 181.7823 |
| LNCaP_2 | 10 | 5 | B | LNCaP | 3307.754 | 0 | 434.7494 | 406.6103 |
| 22RV1_11 | 10 | 5 | B | 22RV1 | 422.9914 | 2.862886 | 388.6368 | 5.010051 |
| DU145_11 | 10 | 5 | B | DU145 | 2160.376 | 45.84041 | 443.124 | 266.8147 |
| PANC10_19 | 20 | 2 | C | PANC10 | 6.672573 | 14.67966 | 898.1284 | 218.8604 |
| PANC10_20 | 20 | 5 | C | PANC10 | 6.763815 | 15.9872 | 863.3087 | 270.5526 |
| LNCaP_11 | 20 | 2 | C | LNCaP | 2048.336 | 1.482154 | 385.3599 | 183.7871 |
| LNCaP_12 | 20 | 5 | C | LNCaP | 1867.387 | 1.741965 | 341.4252 | 209.0358 |
| 22RV1_19 | 20 | 2 | C | 22RV1 | 388.0382 | 6.424474 | 489.5449 | 3.854685 |
| PC3_19 | 20 | 5 | C | PC3 | 448.7851 | 26.50305 | 519.8133 | 1988.082 |
| PC3_22 | 20 | 2 | C | PC3 | 454.248 | 28.50929 | 527.1051 | 2062.805 |
| DU145_19 | 20 | 2 | C | DU145 | 1517.187 | 52.92512 | 459.7221 | 214.8137 |
| DU145_20 | 20 | 5 | C | DU145 | 1555.453 | 59.33068 | 324.3411 | 163.1594 |
| LNCaP_23 | 10 | 2 | A | LNCaP | 1740.446 | 0 | 395.007 | 222.5149 |
| 22RV1_5 | 10 | 2 | A | 22RV1 | 405.2257 | 3.508448 | 440.3102 | 0.877112 |
| PC3_5 | 10 | 2 | A | PC3 | 759.6934 | 52.39265 | 621.007 | 1768.252 |
| DU145_5 | 10 | 2 | A | DU145 | 2189.969 | 97.77537 | 631.5491 | 451.9617 |
| PANC10_14 | 10 | 5 | B | PANC10 | 12.60679 | 15.9686 | 1015.267 | 196.6659 |
| LNCaP_5 | 10 | 5 | B | LNCaP | 2971.391 | 2.617197 | 423.1135 | 247.7613 |
| 22RV1_14 | 10 | 5 | B | 22RV1 | 448.52 | 3.417295 | 387.0087 | 2.562971 |
| PC3_14 | 10 | 5 | B | PC3 | 1152.941 | 83.42015 | 827.3535 | 1848.939 |
| DU145_14 | 10 | 5 | B | DU145 | 2995.52 | 122.0707 | 499.0539 | 581.6312 |
| PANC10_22 | 20 | 2 | C | PANC10 | 8.039135 | 13.84518 | 803.5786 | 181.3272 |
| PANC10_23 | 20 | 5 | C | PANC10 | 12.50035 | 11.41336 | 705.9978 | 197.8316 |
| LNCaP_14 | 20 | 2 | C | LNCaP | 2168.046 | 0 | 295.1601 | 237.8268 |
| LNCaP_15 | 20 | 5 | C | LNCaP | 2407.96 | 0 | 291.3245 | 273.1923 |
| 22RV1_22 | 20 | 2 | C | 22RV1 | 427.6037 | 2.50794 | 408.7942 | 0 |
| 22RV1_23 | 20 | 5 | C | 22RV1 | 399.0337 | 0 | 342.8647 | 7.02112 |
| PC3_20 | 20 | 5 | C | PC3 | 667.6491 | 40.60478 | 574.4578 | 1722.042 |
| PC3_23 | 20 | 2 | C | PC3 | 632.2531 | 49.76066 | 530.2925 | 1614.782 |
| DU145_22 | 20 | 2 | C | DU145 | 1321.005 | 34.14363 | 492.1391 | 250.779 |
| DU145_23 | 20 | 5 | C | DU145 | 1298.832 | 57.50957 | 452.1137 | 222.0754 |
| PANC10_6 | 10 | 2 | A | PANC10 | 15.11898 | 16.79887 | 1069.248 | 212.9256 |
| LNCaP_24 | 10 | 2 | A | LNCaP | 2202.072 | 1.309978 | 421.8128 | 273.7853 |
| 22RV1_8 | 10 | 2 | A | 22RV1 | 425.7942 | 1.517983 | 412.8914 | 3.035966 |
| PC3_8 | 10 | 2 | A | PC3 | 1170.217 | 52.93647 | 982.533 | 2756.707 |
| DU145_8 | 10 | 2 | A | DU145 | 1988.923 | 83.53478 | 681.2063 | 594.6881 |
| PANC10_17 | 10 | 5 | B | PANC10 | 5.104033 | 15.3121 | 1093.397 | 250.0976 |
| LNCaP_8 | 10 | 5 | B | LNCaP | 2407.26 | 0 | 306.1569 | 268.2259 |
| 22RV1_17 | 10 | 5 | B | 22RV1 | 427.1449 | 2.64486 | 407.3084 | 3.96729 |
| PC3_17 | 10 | 5 | B | PC3 | 858.4368 | 53.15173 | 736.115 | 1716.145 |
| DU145_17 | 10 | 5 | B | DU145 | 1856.556 | 97.77131 | 515.2219 | 582.2337 |
| PANC10_26 | 20 | 5 | C | PANC10 | 7.498328 | 11.75414 | 959.3807 | 143.6842 |
| LNCaP_17 | 20 | 2 | C | LNCaP | 2044.425 | 1.269041 | 280.4581 | 291.8795 |
| LNCaP_18 | 20 | 5 | C | LNCaP | 2672.67 | 0 | 345.4538 | 241.9144 |
| 22RV1_25 | 20 | 2 | C | 22RV1 | 390.7094 | 3.412309 | 412.8894 | 3.412309 |
| 22RV1_26 | 20 | 5 | C | 22RV1 | 420.5327 | 1.605087 | 418.9277 | 3.210174 |
| PC3_21 | 20 | 5 | C | PC3 | 500.5573 | 35.04888 | 484.267 | 1771.696 |
| PC3_24 | 20 | 2 | C | PC3 | 491.581 | 24.89636 | 471.0781 | 1757.878 |
| DU145_25 | 20 | 2 | C | DU145 | 1469.877 | 60.67242 | 375.8503 | 206.1983 |
| DU145_26 | 20 | 5 | C | DU145 | 964.7252 | 0 | 241.1813 | 0 |
| PC3_PSI_4 | 20 | 0.5 | A | PC3 | 1100.137 | 90.7685 | 1469.531 | 3095.321 |
| PC3_PSI_7 | 20 | 2 | A | PC3 | 918.0267 | 43.90757 | 1006.738 | 2725.854 |
| PC3_PSI_10 | 20 | 5 | A | PC3 | 907.5433 | 41.77361 | 820.3236 | 2921.858 |
| PC3_PSI_5 | 20 | 0.5 | B | PC3 | 1087.4 | 70.17042 | 1380.18 | 3091.854 |
| PC3_PSI_8 | 20 | 2 | B | PC3 | 913.2592 | 46.1511 | 1042.837 | 2678.539 |
| PC3_PSI_11 | 20 | 5 | B | PC3 | 872.1883 | 46.45264 | 863.7788 | 3215.243 |
| PC3_PSI_6 | 20 | 0.5 | C | PC3 | 1179.131 | 120.4338 | 1625.856 | 2820.392 |
| PC3_PSI_9 | 20 | 2 | C | PC3 | 888.8183 | 68.85212 | 973.8403 | 2787.989 |
| PC3_PSI_12 | 20 | 5 | C | PC3 | 916.3415 | 54.72688 | 1090.533 | 2917.877 |
| PANC10_1 | 1 | 2 | A | PANC10 | 10.44371 | 130.5464 | 2637.037 | 877.2717 |
| LNCaP_19 | 1 | 2 | A | LNCaP | 11168.95 | 2.913893 | 1041.717 | 926.6181 |
| 22RV1_1 | 1 | 2 | A | 22RV1 | 467.7926 | 1.430558 | 702.4041 | 0 |
| PC3_1 | 1 | 2 | A | PC3 | 1877.161 | 114.1301 | 1523.597 | 3456.626 |
| DU145_1 | 1 | 2 | A | DU145 | 4441.451 | 204.9732 | 1043.5 | 1381.103 |
| PANC10_10 | 1 | 5 | B | PANC10 | 10.04338 | 92.62231 | 2322.253 | 790.0795 |
| LNCaP_3 | 1 | 5 | B | LNCaP | 9018.051 | 6.091631 | 877.1949 | 565.3034 |
| 22RV1_10 | 1 | 5 | B | 22RV1 | 2023.365 | 16.86138 | 919.5071 | 21.35775 |
| DU145_10 | 1 | 5 | B | DU145 | 4712.332 | 162.5827 | 842.8631 | 1135.512 |
| PANC10_2 | 1 | 2 | A | PANC10 | 11.57364 | 82.02187 | 3135.449 | 946.5223 |
| 22RV1_4 | 1 | 2 | A | 22RV1 | 1756.816 | 22.55541 | 1064.281 | 11.6954 |
| PC3_4 | 1 | 2 | A | PC3 | 5423.365 | 169.4506 | 1770.238 | 5201.848 |
| DU145_4 | 1 | 2 | A | DU145 | 4238.848 | 313.1314 | 1129.466 | 1706.992 |
| PANC10_13 | 1 | 5 | B | PANC10 | 6.359951 | 131.7418 | 2111.504 | 914.9243 |
| LNCaP_6 | 1 | 5 | B | LNCaP | 8195.015 | 6.04263 | 861.679 | 576.4669 |
| 22RV1_13 | 1 | 5 | B | 22RV1 | 1859.612 | 20.94158 | 894.2053 | 32.45944 |
| PC3_13 | 1 | 5 | B | PC3 | 4191.345 | 268.4914 | 2037.781 | 5547.57 |
| DU145_13 | 1 | 5 | B | DU145 | 5865.239 | 364.627 | 652.0583 | 1364.888 |
| PANC10_3 | 1 | 2 | A | PANC10 | 5.984618 | 133.1578 | 2558.424 | 951.5543 |
| LNCaP_21 | 1 | 2 | A | LNCaP | 8482.947 | 2.823884 | 1042.013 | 883.8756 |
| 22RV1_7 | 1 | 2 | A | 22RV1 | 3168.179 | 34.55534 | 1164.879 | 24.55248 |
| PC3_7 | 1 | 2 | A | PC3 | 5787.271 | 200.7905 | 2149.898 | 6611.701 |
| DU145_7 | 1 | 2 | A | DU145 | 4169.23 | 240.674 | 1067.794 | 1692.075 |
| PANC10_16 | 1 | 5 | B | PANC10 | 18.07991 | 142.5122 | 2173.843 | 1168.813 |
| LNCaP_9 | 1 | 5 | B | LNCaP | 7559.562 | 6.743587 | 987.9356 | 560.8417 |
| 22RV1_16 | 1 | 5 | B | 22RV1 | 1931.307 | 14.18093 | 937.2917 | 31.06298 |
| PC3_16 | 1 | 5 | B | PC3 | 4419.143 | 254.2238 | 1826.518 | 4920.72 |
| DU145_16 | 1 | 5 | B | DU145 | 8228.795 | 590.7299 | 304.37 | 633.954 |

TABLE 14
Pressure-Sensitive Candidates

| | O2 | PSI | Expt | CellLine | RBM3 | CBX3P2 | TMEM260 | TBCK | COX11 | FBXO36 | ACBD6 | EXOSC9 |
|---|---|---|---|---|---|---|---|---|---|---|---|---|
| LNCaP_25 | 20 | 0 | A | LNCaP | 2341.237 | 20.29974 | 228.3721 | 221.6055 | 676.6581 | 42.29113 | 338.329 | 605.609 |
| 22RV1_3 | 20 | 0 | A | 22RV1 | 3323.104 | 4.018264 | 225.0228 | 117.8691 | 409.8629 | 121.8873 | 543.8051 | 692.4809 |
| PC3_3 | 20 | 0 | A | PC3 | 4555.265 | 7.581416 | 397.1495 | 82.2292 | 964.0062 | 51.32035 | 285.7611 | 415.8115 |
| DU145_3 | 20 | 0 | A | DU145 | 4080.535 | 15.31158 | 320.4494 | 79.83894 | 926.3505 | 29.52947 | 530.4368 | 692.3021 |
| PANC10_8 | 20 | 0 | A | PANC10 | 6270.177 | 0.606048 | 235.7528 | 93.33146 | 307.2666 | 54.54436 | 1032.101 | 522.4138 |
| LNCaP_26 | 20 | 0 | A | LNCaP | 2751.567 | 25.54505 | 242.678 | 215.3083 | 824.7403 | 58.3887 | 326.6118 | 571.1144 |
| 22RV1_6 | 20 | 0 | A | 22RV1 | 2628.915 | 6.161519 | 216.6801 | 138.6342 | 417.9564 | 137.6073 | 546.3213 | 668.5248 |
| PC3_6 | 20 | 0 | A | PC3 | 4268.403 | 5.851134 | 368.6215 | 136.7703 | 1071.489 | 65.09387 | 277.9289 | 398.6085 |
| DU145_6 | 20 | 0 | A | DU145 | 4347.829 | 12.66246 | 279.7253 | 90.93949 | 920.9063 | 18.41813 | 569.8107 | 626.2163 |
| PANC10_9 | 20 | 0 | A | PANC10 | 5259.65 | 0 | 249.9957 | 126.6212 | 288.9561 | 25.97358 | 922.0621 | 461.031 |
| LNCaP_27 | 20 | 0 | A | LNCaP | 2539.816 | 34.26736 | 255.9974 | 203.5885 | 691.3944 | 28.22018 | 336.6264 | 554.325 |
| 22RV1_9 | 20 | 0 | A | 22RV1 | 2625.388 | 6.158065 | 221.6903 | 131.3721 | 359.2205 | 143.6882 | 582.9635 | 689.7033 |
| PC3_9 | 20 | 0 | A | PC3 | 3764.649 | 4.478393 | 378.4242 | 124.2754 | 1153.746 | 75.01308 | 274.8614 | 372.8262 |
| DU145_9 | 20 | 0 | A | DU145 | 3828.298 | 1.100086 | 361.9281 | 116.6091 | 1103.386 | 19.80154 | 561.0436 | 597.3464 |
| PC3_PSI_1 | 20 | 0 | A | PC3 | 3345.699 | 18.75314 | 585.5147 | 181.9749 | 1030.728 | 113.908 | 508.4185 | 203.5063 |
| PANC10_12 | 20 | 0 | B | PANC10 | 6561.966 | 4.168974 | 244.5798 | 75.04154 | 286.2696 | 66.70359 | 926.9019 | 397.4422 |
| LNCaP_1 | 20 | 0 | B | LNCaP | 2565.552 | 24.9744 | 296.2872 | 228.1752 | 751.5025 | 44.27281 | 333.7488 | 643.6585 |
| 22RV1_12 | 20 | 0 | B | 22RV1 | 2731.123 | 7.128543 | 272.6668 | 145.2441 | 412.5644 | 127.4227 | 539.9871 | 676.3205 |
| DU145_12 | 20 | 0 | B | DU145 | 6787.653 | 10.19507 | 317.18 | 138.1999 | 1218.878 | 44.17865 | 456.5127 | 872.2451 |
| PANC10_15 | 20 | 0 | B | PANC10 | 5285.68 | 5.69087 | 227.6348 | 138.8572 | 293.6489 | 28.45435 | 1040.291 | 452.9933 |
| LNCaP_4 | 20 | 0 | B | LNCaP | 2600.395 | 16.17664 | 203.5561 | 188.7275 | 644.3695 | 52.57408 | 358.5822 | 474.5148 |
| 22RV1_15 | 20 | 0 | B | 22RV1 | 2673.805 | 3.899096 | 251.4917 | 147.1909 | 423.0519 | 125.7458 | 530.277 | 669.6697 |
| PC3_15 | 20 | 0 | B | PC3 | 2805.644 | 10.84726 | 374.7729 | 129.6248 | 997.9482 | 71.59193 | 258.1648 | 391.0438 |
| DU145_15 | 20 | 0 | B | DU145 | 4734.228 | 9.073265 | 275.2224 | 104.8466 | 858.9358 | 31.25236 | 499.0296 | 725.8612 |
| PANC10_18 | 20 | 0 | B | PANC10 | 4834.155 | 1.643711 | 259.7064 | 128.2095 | 386.2722 | 75.61073 | 943.4904 | 460.2392 |
| LNCaP_7 | 20 | 0 | B | LNCaP | 3271.787 | 40.77971 | 240.4957 | 174.6208 | 737.1716 | 37.64281 | 388.9757 | 673.388 |
| 22RV1_18 | 20 | 0 | B | 22RV1 | 2480.712 | 0 | 296.6069 | 118.1292 | 403.18 | 116.8452 | 463.5286 | 680.5267 |
| PC3_18 | 20 | 0 | B | PC3 | 3387.732 | 10.82559 | 347.0953 | 169.1498 | 1035.873 | 58.18752 | 288.2312 | 374.1593 |
| DU145_18 | 20 | 0 | B | DU145 | 3878.265 | 9.042353 | 272.1748 | 134.7311 | 993.7546 | 41.59482 | 646.5282 | 697.1654 |
| PC3_PSI_2 | 20 | 0 | B | PC3 | 4171.552 | 19.71122 | 517.7481 | 95.27091 | 949.4239 | 122.2096 | 739.8279 | 224.0509 |
| PANC10_21 | 20 | 0 | C | PANC10 | 6169.241 | 5.299656 | 268.0743 | 135.1412 | 317.9793 | 61.82932 | 1016.651 | 535.7069 |
| LNCaP_10 | 20 | 0 | C | LNCaP | 3535.704 | 18.98767 | 196.8721 | 163.8936 | 670.5645 | 58.96171 | 354.7696 | 599.6106 |
| 22RV1_21 | 20 | 0 | C | 22RV1 | 3391.47 | 12.93108 | 228.0572 | 124.6086 | 410.2679 | 136.3641 | 557.212 | 618.3408 |
| PC3_25 | 20 | 0 | C | PC3 | 3779.403 | 14.04676 | 329.6856 | 147.0778 | 1022.934 | 69.4075 | 303.2447 | 444.5385 |
| DU145_21 | 20 | 0 | C | DU145 | 4444.594 | 2.019352 | 268.5738 | 122.1708 | 1058.14 | 33.31931 | 698.6958 | 657.2991 |
| PANC10_24 | 20 | 0 | C | PANC10 | 6779.829 | 1.364151 | 243.7283 | 105.949 | 321.4849 | 50.92829 | 866.2357 | 544.7509 |
| LNCaP_13 | 20 | 0 | C | LNCaP | 3172.538 | 24.7119 | 182.3974 | 167.0995 | 728.4128 | 48.24705 | 358.911 | 487.1775 |
| 22RV1_24 | 20 | 0 | C | 22RV1 | 2659.845 | 5.064763 | 244.7969 | 152.787 | 405.1811 | 157.8518 | 522.5148 | 608.6157 |
| PC3_26 | 20 | 0 | C | PC3 | 3815.104 | 10.00935 | 309.52 | 130.8916 | 942.4193 | 62.36598 | 322.6092 | 448.8811 |
| DU145_24 | 20 | 0 | C | DU145 | 3565.923 | 8.100688 | 251.1213 | 132.8513 | 852.1924 | 43.74372 | 665.8766 | 638.3342 |
| PANC10_27 | 20 | 0 | C | PANC10 | 5134.69 | 3.428841 | 183.443 | 132.0104 | 294.8803 | 39.43167 | 872.6401 | 375.4581 |
| LNCaP_16 | 20 | 0 | C | LNCaP | 3575.29 | 19.86962 | 194.9707 | 166.4081 | 613.4746 | 45.9485 | 356.4114 | 500.4661 |
| 22RV1_27 | 20 | 0 | C | 22RV1 | 2834.926 | 5.064176 | 231.9393 | 131.6686 | 408.1726 | 127.6172 | 508.4433 | 645.176 |
| PC3_27 | 20 | 0 | C | PC3 | 3813.55 | 12.46687 | 361.5393 | 154.8517 | 1049.186 | 84.6435 | 299.2049 | 374.0061 |
| DU145_27 | 20 | 0 | C | DU145 | 2988.566 | 0 | 110.6876 | 442.7505 | 1217.564 | 0 | 885.5009 | 221.3752 |
| PC3_PSI_3 | 20 | 0 | C | PC3 | 3076.486 | 13.53176 | 567.5381 | 179.8929 | 992.5947 | 144.0735 | 473.6117 | 220.4881 |
| PANC10_4 | 10 | 2 | A | PANC10 | 6386.911 | 1.135451 | 172.5885 | 70.39795 | 373.5633 | 44.28258 | 1051.428 | 463.264 |
| PC3_2 | 10 | 2 | A | PC3 | 4264.816 | 16.93546 | 345.7169 | 85.26127 | 1086.205 | 85.26127 | 321.7737 | 476.5288 |
| DU145_2 | 10 | 2 | A | DU145 | 5171.311 | 40.9894 | 187.2218 | 62.03801 | 1350.435 | 73.11622 | 820.8958 | 1113.361 |
| LNCaP_23 | 10 | 2 | A | LNCaP | 6032.05 | 53.47256 | 196.641 | 134.5439 | 705.4928 | 60.37225 | 464.0038 | 679.619 |
| 22RV1_5 | 10 | 2 | A | 22RV1 | 5267.057 | 18.41935 | 207.8755 | 102.6221 | 545.5636 | 175.4224 | 621.8724 | 892.8999 |
| PC3_5 | 10 | 2 | A | PC3 | 5504.31 | 7.704801 | 338.2408 | 91.68713 | 1168.048 | 85.52329 | 336.6998 | 506.2054 |
| DU145_5 | 10 | 2 | A | DU145 | 6664.689 | 49.88539 | 160.631 | 51.88081 | 1336.929 | 83.80746 | 832.0884 | 1152.353 |
| PANC10_6 | 10 | 2 | A | PANC10 | 7171.436 | 6.719546 | 202.8463 | 88.19404 | 308.6792 | 103.733 | 1004.992 | 487.5871 |
| LNCaP_24 | 10 | 2 | A | LNCaP | 6097.945 | 69.42881 | 182.0869 | 142.7876 | 865.8952 | 94.31838 | 427.0527 | 685.1183 |
| 22RV1_8 | 10 | 2 | A | 22RV1 | 6350.482 | 13.66185 | 184.4349 | 98.66889 | 553.3048 | 172.2911 | 617.0601 | 900.9229 |
| PC3_8 | 10 | 2 | A | PC3 | 4895.822 | 13.63515 | 320.025 | 119.5081 | 1160.592 | 82.61298 | 361.7326 | 405.8463 |
| DU145_8 | 10 | 2 | A | DU145 | 6087.1 | 52.70647 | 264.5268 | 81.54586 | 1264.955 | 48.72862 | 782.6413 | 959.6555 |
| PC3_PSI_4 | 20 | 0.5 | A | PC3 | 4525.786 | 36.1925 | 521.6316 | 144.1955 | 994.4321 | 145.919 | 503.8226 | 238.9854 |
| PC3_PSI_7 | 20 | 2 | A | PC3 | 8340.647 | 52.42027 | 316.7618 | 69.44565 | 1302.89 | 161.7412 | 813.1862 | 346.7802 |
| PC3_PSI_10 | 20 | 5 | A | PC3 | 7532.104 | 33.96975 | 339.2385 | 64.72615 | 1331.247 | 147.3553 | 691.3304 | 316.2859 |
| PANC10_11 | 10 | 5 | B | PANC10 | 6243.027 | 2.754277 | 179.9461 | 80.79211 | 329.5951 | 76.20165 | 1043.871 | 448.9471 |
| LNCaP_2 | 10 | 5 | B | LNCaP | 5325.329 | 26.73217 | 175.8695 | 112.5565 | 699.2572 | 68.94085 | 448.819 | 583.8868 |
| 22RV1_11 | 10 | 5 | B | 22RV1 | 3995.158 | 9.30438 | 193.2448 | 106.6425 | 491.7007 | 130.977 | 642.0022 | 775.8422 |
| DU145_11 | 10 | 5 | B | DU145 | 9470.159 | 52.89278 | 228.0267 | 106.961 | 1691.394 | 128.1181 | 584.1714 | 1290.584 |
| PANC10_14 | 10 | 5 | B | PANC10 | 6439.549 | 6.723622 | 181.5378 | 82.36437 | 305.0843 | 90.76889 | 1093.429 | 451.3231 |
| LNCaP_5 | 10 | 5 | B | LNCaP | 6890.206 | 63.68512 | 225.0789 | 134.3494 | 1019.834 | 81.1331 | 418.7515 | 727.5807 |
| 22RV1_14 | 10 | 5 | B | 22RV1 | 4622.746 | 11.10621 | 210.1637 | 109.3534 | 473.2954 | 134.9832 | 633.054 | 762.9112 |
| PC3_14 | 10 | 5 | B | PC3 | 4628.573 | 14.94092 | 275.162 | 75.32715 | 1221.42 | 89.64553 | 369.1653 | 520.4421 |
| DU145_14 | 10 | 5 | B | DU145 | 12797.08 | 55.05151 | 192.6803 | 71.80632 | 1505.539 | 106.5127 | 578.0409 | 1055.553 |
| PANC10_17 | 10 | 5 | B | PANC10 | 5897.427 | 2.835574 | 199.0573 | 86.76856 | 395.8461 | 67.48666 | 1159.183 | 498.4939 |
| LNCaP_8 | 10 | 5 | B | LNCaP | 5078.682 | 37.93094 | 188.3 | 125.9849 | 800.6138 | 75.86188 | 466.0087 | 606.8951 |
| 22RV1_17 | 10 | 5 | B | 22RV1 | 3329.879 | 9.25701 | 259.1963 | 100.5047 | 403.3411 | 148.1122 | 536.9066 | 720.7243 |
| PC3_17 | 10 | 5 | B | PC3 | 5029.755 | 11.64969 | 297.7953 | 112.8564 | 1199.19 | 74.9949 | 363.3248 | 450.6975 |
| DU145_17 | 10 | 5 | B | DU145 | 6037.653 | 24.16819 | 253.766 | 110.954 | 1286.407 | 48.33638 | 781.0719 | 853.5765 |
| PC3_PSI_5 | 20 | 0.5 | B | PC3 | 4611.407 | 27.1003 | 517.8093 | 139.373 | 1029.328 | 146.1481 | 630.082 | 248.2581 |
| PC3_PSI_8 | 20 | 2 | B | PC3 | 8268.147 | 33.7258 | 360.3336 | 72.77673 | 1231.879 | 170.4041 | 657.6531 | 305.3073 |
| PC3_PSI_11 | 20 | 5 | B | PC3 | 7663.484 | 32.0363 | 309.5508 | 67.67669 | 1285.457 | 151.3715 | 676.3664 | 354.0011 |
| PANC10_19 | 20 | 2 | C | PANC10 | 6286.898 | 8.007088 | 149.4656 | 72.06379 | 344.3048 | 109.4302 | 1024.907 | 464.4111 |
| PANC10_20 | 20 | 5 | C | PANC10 | 5962.61 | 7.378707 | 135.8912 | 77.47642 | 336.9609 | 91.61894 | 988.7467 | 509.7457 |
| LNCaP_11 | 20 | 2 | C | LNCaP | 5936.025 | 74.10768 | 123.0188 | 105.2329 | 812.2202 | 111.1615 | 545.4325 | 690.6836 |
| LNCaP_12 | 20 | 5 | C | LNCaP | 4783.436 | 38.32323 | 184.6483 | 146.3251 | 769.9486 | 73.16253 | 426.7815 | 661.9467 |
| 22RV1_19 | 20 | 2 | C | 22RV1 | 4621.767 | 16.70363 | 156.7572 | 104.0765 | 535.8011 | 179.8853 | 719.5411 | 846.7457 |
| PC3_19 | 20 | 5 | C | PC3 | 4385.726 | 15.90183 | 302.1348 | 111.3128 | 1166.488 | 111.3128 | 318.39 | 461.5065 |
| PC3_22 | 20 | 2 | C | PC3 | 5214.032 | 23.44097 | 259.1178 | 104.5341 | 1242.371 | 128.6086 | 333.8754 | 611.9994 |
| DU145_19 | 20 | 2 | C | DU145 | 5413.928 | 16.60396 | 189.9078 | 64.34034 | 1230.768 | 47.73638 | 777.2728 | 801.141 |
| DU145_20 | 20 | 5 | C | DU145 | 6244.554 | 21.75458 | 173.0478 | 78.11873 | 1342.851 | 51.41992 | 997.7443 | 972.0343 |
| PANC10_22 | 20 | 2 | C | PANC10 | 7881.479 | 8.709063 | 133.204 | 64.31308 | 340.3234 | 104.0621 | 1066.749 | 517.1844 |
| PANC10_23 | 20 | 5 | C | PANC10 | 10103.54 | 11.41336 | 109.7856 | 52.71885 | 382.0758 | 135.8733 | 1322.863 | 652.192 |
| LNCaP_14 | 20 | 2 | C | LNCaP | 4922.166 | 37.16044 | 170.938 | 128.469 | 864.2458 | 57.33326 | 455.4809 | 587.135 |
| LNCaP_15 | 20 | 5 | C | LNCaP | 5734.62 | 37.47328 | 198.2457 | 161.9813 | 934.4143 | 59.23196 | 442.4264 | 685.3983 |
| 22RV1_22 | 20 | 2 | C | 22RV1 | 4016.466 | 11.28573 | 208.159 | 116.6192 | 448.9212 | 152.9843 | 560.5245 | 753.6359 |
| 22RV1_23 | 20 | 5 | C | 22RV1 | 4193.949 | 7.02112 | 208.2932 | 106.487 | 463.3939 | 161.4858 | 599.1356 | 753.6002 |
| PC3_20 | 20 | 5 | C | PC3 | 4357.359 | 9.31913 | 295.5495 | 114.4922 | 1065.043 | 102.5104 | 340.8139 | 451.3121 |
| PC3_23 | 20 | 2 | C | PC3 | 4707.749 | 18.05044 | 255.6332 | 97.56992 | 1064.976 | 103.912 | 384.4255 | 515.657 |
| DU145_22 | 20 | 2 | C | DU145 | 4967.309 | 18.83786 | 289.6321 | 83.59301 | 1186.785 | 28.25679 | 676.9857 | 766.4655 |
| DU145_23 | 20 | 5 | C | DU145 | 5566.042 | 17.69525 | 282.2393 | 107.0563 | 1304.14 | 45.1229 | 820.175 | 728.1597 |
| PANC10_26 | 20 | 5 | C | PANC10 | 8129.403 | 9.119588 | 163.7473 | 72.34873 | 346.5443 | 113.4882 | 1121.507 | 516.1687 |
| LNCaP_17 | 20 | 2 | C | LNCaP | 6488.608 | 32.99507 | 156.0921 | 110.4066 | 736.0439 | 72.33535 | 445.4335 | 588.8351 |
| LNCaP_18 | 20 | 5 | C | LNCaP | 7181.955 | 52.25351 | 197.4021 | 138.375 | 1079.906 | 64.83306 | 566.0797 | 755.7406 |
| 22RV1_25 | 20 | 2 | C | 22RV1 | 5585.949 | 20.47385 | 206.4447 | 98.95695 | 440.1878 | 170.6154 | 590.3294 | 766.0633 |
| 22RV1_26 | 20 | 5 | C | 22RV1 | 5535.944 | 9.630521 | 195.8206 | 104.3306 | 455.8446 | 216.6867 | 563.3855 | 781.6773 |
| PC3_21 | 20 | 5 | C | PC3 | 4587.455 | 12.34116 | 311.4908 | 131.3099 | 1080.592 | 81.94528 | 348.5143 | 459.091 |
| PC3_24 | 20 | 2 | C | PC3 | 4373.948 | 14.64492 | 308.0314 | 144.4965 | 1114.478 | 87.86949 | 330.9751 | 424.2144 |
| DU145_25 | 20 | 2 | C | DU145 | 6117.638 | 22.03772 | 206.3632 | 86.722 | 1293.521 | 36.49138 | 824.7932 | 820.0669 |
| DU145_26 | 20 | 5 | C | DU145 | 2894.176 | 0 | 241.1813 | 0 | 241.1813 | 0 | 0 | 2652.994 |
| PC3_PSI_6 | 20 | 0.5 | C | PC3 | 4629.7 | 26.60747 | 476.1337 | 166.6468 | 981.6756 | 166.6468 | 674.9895 | 253.4712 |
| PC3_PSI_9 | 20 | 2 | C | PC3 | 7738.04 | 31.81803 | 320.7883 | 68.85212 | 1257.594 | 162.7414 | 730.2498 | 336.4365 |
| PC3_PSI_12 | 20 | 5 | C | PC3 | 6790.805 | 28.69824 | 326.3591 | 79.42071 | 1058.832 | 142.8238 | 721.7941 | 265.9593 |
| PANC10_1 | 1 | 2 | A | PANC10 | 6542.985 | 15.66557 | 177.5431 | 31.33113 | 344.6425 | 20.88742 | 1206.249 | 386.4173 |
| LNCaP_19 | 1 | 2 | A | LNCaP | 6474.671 | 48.07924 | 179.2044 | 88.87375 | 687.6788 | 67.01955 | 543.4411 | 619.2023 |
| 22RV1_1 | 1 | 2 | A | 22RV1 | 4530.578 | 25.75005 | 167.3753 | 90.12517 | 497.8343 | 175.9587 | 716.7097 | 1032.863 |
| PC3_1 | 1 | 2 | A | PC3 | 5083.18 | 13.56792 | 287.3206 | 63.84902 | 1140.503 | 91.78297 | 339.996 | 620.1336 |
| DU145_1 | 1 | 2 | A | DU145 | 4611.349 | 23.01838 | 203.8771 | 76.72793 | 1121.324 | 52.61344 | 643.4185 | 842.9111 |
| PANC10_2 | 1 | 2 | A | PANC10 | 9006.806 | 11.57364 | 198.2614 | 81.51866 | 371.3628 | 150.9605 | 1150.822 | 517.7945 |
| 22RV1_4 | 1 | 2 | A | 22RV1 | 6490.111 | 11.6954 | 183.7848 | 99.41089 | 521.2806 | 182.9495 | 675.827 | 860.4472 |
| PC3_4 | 1 | 2 | A | PC3 | 6749.623 | 17.98637 | 247.076 | 81.412 | 1196.567 | 88.03856 | 360.6741 | 476.1655 |
| DU145_4 | 1 | 2 | A | DU145 | 5878.828 | 43.86276 | 194.9456 | 86.50711 | 1046.614 | 71.88619 | 661.5966 | 952.7966 |
| PANC10_3 | 1 | 2 | A | PANC10 | 7205.48 | 7.480773 | 187.0193 | 71.81542 | 275.2924 | 112.2116 | 975.4928 | 409.9463 |
| LNCaP_21 | 1 | 2 | A | LNCaP | 6624.831 | 31.06272 | 189.2002 | 121.427 | 796.3352 | 50.82991 | 454.6453 | 810.4547 |
| 22RV1_7 | 1 | 2 | A | 22RV1 | 6886.516 | 12.73092 | 185.5076 | 92.75382 | 509.2367 | 176.4141 | 649.2767 | 845.6966 |
| PC3_7 | 1 | 2 | A | PC3 | 6622.96 | 18.76547 | 282.7331 | 78.18946 | 1148.447 | 76.31292 | 363.4246 | 402.8321 |
| DU145_7 | 1 | 2 | A | DU145 | 6479.28 | 55.70184 | 243.8269 | 102.9958 | 1239.103 | 58.85477 | 715.716 | 903.8411 |
| PANC10_10 | 1 | 5 | B | PANC10 | 6992.427 | 6.695589 | 189.7084 | 70.30368 | 302.4174 | 104.8976 | 1035.584 | 464.2275 |
| LNCaP_3 | 1 | 5 | B | LNCaP | 4050.935 | 20.71155 | 221.7354 | 194.9322 | 670.0794 | 70.66292 | 397.1744 | 556.7751 |
| 22RV1_10 | 1 | 5 | B | 22RV1 | 3566.743 | 13.4891 | 256.2929 | 130.3947 | 465.374 | 145.0078 | 537.3159 | 657.5937 |
| DU145_10 | 1 | 5 | B | DU145 | 7010.738 | 26.52666 | 258.421 | 85.56986 | 1233.917 | 72.73438 | 604.9789 | 1029.405 |
| PANC10_13 | 1 | 5 | B | PANC10 | 7888.156 | 3.634258 | 158.9988 | 53.6053 | 268.9351 | 99.94208 | 1051.209 | 480.6306 |
| LNCaP_6 | 1 | 5 | B | LNCaP | 4510.219 | 31.42167 | 209.075 | 196.9897 | 685.2342 | 51.96662 | 404.8562 | 663.4808 |
| 22RV1_13 | 1 | 5 | B | 22RV1 | 4500.345 | 13.61203 | 276.4288 | 110.9904 | 468.0443 | 127.7436 | 639.7652 | 774.8384 |
| PC3_13 | 1 | 5 | B | PC3 | 5296.603 | 20.02733 | 262.2329 | 83.23859 | 1182.238 | 103.2659 | 369.2539 | 473.7715 |
| DU145_13 | 1 | 5 | B | DU145 | 10515.06 | 27.92189 | 192.1683 | 54.20132 | 1005.188 | 93.62045 | 796.5951 | 850.7964 |
| PANC10_16 | 1 | 5 | B | PANC10 | 5799.396 | 17.01638 | 226.5306 | 57.43029 | 307.3584 | 82.95487 | 916.7577 | 398.8215 |
| LNCaP_9 | 1 | 5 | B | LNCaP | 4866.622 | 33.71794 | 204.5555 | 176.4572 | 904.7646 | 60.69229 | 400.1195 | 714.8203 |
| 22RV1_16 | 1 | 5 | B | 22RV1 | 3998.346 | 9.453951 | 249.8544 | 94.53951 | 292.3972 | 145.1857 | 644.8945 | 759.0172 |
| PC3_16 | 1 | 5 | B | PC3 | 5563.723 | 20.61274 | 300.0299 | 92.18477 | 1156.031 | 75.58006 | 368.7391 | 382.4809 |
| DU145_16 | 1 | 5 | B | DU145 | 8976.212 | 7.204023 | 198.1106 | 59.43319 | 1197.669 | 5.403017 | 1298.525 | 617.7449 |

TABLE 15
Pressure-Sensitive Candidates

| | O2 | PSI | Expt | CellLine | SMYD3 | EIF4A2 | DDHD2 | PDIA5 | COPG2 | TNFRSF13C | SLC6A6 | NRAV |
|---|---|---|---|---|---|---|---|---|---|---|---|---|
| LNCaP_25 | 20 | 0 | A | LNCaP | 216.5306 | 4092.09 | 707.1077 | 466.8941 | 776.4651 | 16.91645 | 159.0146 | 209.764 |
| 22RV1_3 | 20 | 0 | A | 22RV1 | 226.3622 | 2118.965 | 530.4109 | 324.14 | 358.9649 | 57.59512 | 349.589 | 322.8006 |
| PC3_3 | 20 | 0 | A | PC3 | 301.5071 | 2661.077 | 534.7814 | 355.1602 | 315.5035 | 2.915929 | 1007.162 | 145.2133 |
| DU145_3 | 20 | 0 | A | DU145 | 398.101 | 4582.536 | 999.6273 | 230.7674 | 550.1231 | 4.374737 | 1817.703 | 283.2642 |
| PANC10_8 | 20 | 0 | A | PANC10 | 247.8738 | 3734.471 | 274.54 | 177.5722 | 453.3242 | 0.606048 | 451.5061 | 223.0258 |
| LNCaP_26 | 20 | 0 | A | LNCaP | 191.5879 | 4698.465 | 738.9819 | 479.8821 | 773.6502 | 7.298587 | 120.4267 | 193.4126 |
| 22RV1_6 | 20 | 0 | A | 22RV1 | 234.1377 | 1957.309 | 602.8019 | 281.376 | 379.9603 | 83.1805 | 337.8566 | 255.703 |
| PC3_6 | 20 | 0 | A | PC3 | 310.1101 | 3292.726 | 553.6636 | 399.3399 | 372.2784 | 3.656959 | 895.955 | 130.9191 |
| DU145_6 | 20 | 0 | A | DU145 | 377.5716 | 4293.725 | 1016.45 | 212.9596 | 572.113 | 6.906797 | 1722.095 | 310.8059 |
| PANC10_9 | 20 | 0 | A | PANC10 | 149.3481 | 3389.552 | 334.4098 | 185.0618 | 444.7976 | 0 | 389.6037 | 204.5419 |
| LNCaP_27 | 20 | 0 | A | LNCaP | 268.0917 | 3942.762 | 608.7496 | 544.2464 | 741.7876 | 8.062909 | 133.038 | 237.8558 |
| 22RV1_9 | 20 | 0 | A | 22RV1 | 254.5334 | 2052.688 | 578.8581 | 328.4301 | 387.9581 | 59.52796 | 398.2215 | 240.1645 |
| PC3_9 | 20 | 0 | A | PC3 | 346.5156 | 3502.103 | 538.5267 | 361.6302 | 401.376 | 3.918594 | 870.4876 | 130.4332 |
| DU145_9 | 20 | 0 | A | DU145 | 375.1292 | 3681.986 | 1170.491 | 178.2139 | 694.154 | 7.700599 | 2293.678 | 276.1215 |
| PC3_PSI_1 | 20 | 0 | A | PC3 | 557.0377 | 5043.206 | 582.7365 | 510.5022 | 357.0042 | 4.861925 | 977.247 | 120.159 |
| PANC10_12 | 20 | 0 | B | PANC10 | 198.7211 | 2358.25 | 244.5798 | 177.8762 | 484.9907 | 2.779316 | 518.3425 | 201.5004 |
| LNCaP_1 | 20 | 0 | B | LNCaP | 211.1472 | 4754.218 | 711.7705 | 463.1617 | 779.8825 | 14.7576 | 148.7112 | 174.8208 |
| 22RV1_12 | 20 | 0 | B | 22RV1 | 210.292 | 2571.622 | 549.7889 | 320.7844 | 372.4664 | 65.93902 | 400.0895 | 326.1308 |
| DU145_12 | 20 | 0 | B | DU145 | 449.716 | 6546.37 | 835.996 | 164.254 | 408.9357 | 5.663929 | 1144.114 | 306.985 |
| PANC10_15 | 20 | 0 | B | PANC10 | 201.4568 | 3136.808 | 349.4194 | 188.9369 | 503.0729 | 2.276348 | 413.1572 | 180.9697 |
| LNCaP_4 | 20 | 0 | B | LNCaP | 243.9977 | 3823.079 | 562.1383 | 571.5746 | 757.606 | 13.48053 | 136.1534 | 186.0314 |
| 22RV1_15 | 20 | 0 | B | 22RV1 | 211.526 | 2394.045 | 582.9148 | 311.9277 | 357.742 | 57.51166 | 386.9853 | 297.3061 |
| PC3_15 | 20 | 0 | B | PC3 | 319.4519 | 2975.404 | 522.8381 | 355.7902 | 319.4519 | 3.796542 | 984.9314 | 167.5902 |
| DU145_15 | 20 | 0 | B | DU145 | 377.0446 | 4683.821 | 805.5043 | 188.5223 | 523.225 | 3.024422 | 1185.573 | 243.97 |
| PANC10_18 | 20 | 0 | B | PANC10 | 190.6705 | 3178.938 | 317.2363 | 184.0957 | 567.0805 | 1.643711 | 427.365 | 198.8891 |
| LNCaP_7 | 20 | 0 | B | LNCaP | 220.6287 | 4329.968 | 680.7074 | 508.1779 | 775.8601 | 3.1369 | 93.06138 | 181.9402 |
| 22RV1_18 | 20 | 0 | B | 22RV1 | 225.9862 | 1863.102 | 626.5982 | 306.879 | 340.2634 | 48.79248 | 430.1442 | 273.4947 |
| PC3_18 | 20 | 0 | B | PC3 | 349.1251 | 3015.602 | 569.6964 | 374.1593 | 349.1251 | 4.736194 | 847.1021 | 135.9964 |
| DU145_18 | 20 | 0 | B | DU145 | 406.9059 | 3251.63 | 768.6 | 226.0588 | 503.6591 | 15.372 | 1249.653 | 245.952 |
| PC3_PSI_2 | 20 | 0 | B | PC3 | 629.4451 | 5833.865 | 511.8348 | 660.983 | 308.1521 | 2.628163 | 599.2212 | 164.2602 |
| PANC10_21 | 20 | 0 | C | PANC10 | 219.9357 | 3093.674 | 298.9889 | 173.1221 | 557.7888 | 2.649828 | 340.9445 | 158.1064 |
| LNCaP_10 | 20 | 0 | C | LNCaP | 286.8137 | 3509.721 | 533.6534 | 576.6255 | 826.4632 | 9.993509 | 147.9039 | 196.8721 |
| 22RV1_21 | 20 | 0 | C | 22RV1 | 186.9129 | 1934.96 | 625.3941 | 345.6125 | 403.2146 | 70.53317 | 237.4617 | 303.2926 |
| PC3_25 | 20 | 0 | C | PC3 | 334.6433 | 2751.511 | 627.9726 | 356.9528 | 349.5163 | 0.82628 | 581.7009 | 140.4676 |
| DU145_21 | 20 | 0 | C | DU145 | 350.3576 | 4212.368 | 971.3083 | 209.0029 | 442.2381 | 5.04838 | 1489.272 | 284.7286 |
| PANC10_24 | 20 | 0 | C | PANC10 | 200.9849 | 2964.754 | 299.2037 | 185.5245 | 438.8018 | 3.637735 | 426.0697 | 188.7075 |
| LNCaP_13 | 20 | 0 | C | LNCaP | 218.8769 | 3543.216 | 601.323 | 557.783 | 776.6598 | 11.76757 | 85.90328 | 245.9423 |
| 22RV1_24 | 20 | 0 | C | 22RV1 | 178.955 | 2172.783 | 612.8364 | 324.989 | 344.4039 | 54.02414 | 277.7179 | 266.7442 |
| PC3_26 | 20 | 0 | C | PC3 | 307.9801 | 2208.218 | 486.6086 | 331.0787 | 298.7407 | 2.309851 | 722.2134 | 145.5206 |
| DU145_24 | 20 | 0 | C | DU145 | 325.6477 | 3784.642 | 821.4098 | 223.579 | 429.3365 | 12.9611 | 1370.636 | 262.4623 |
| PANC10_27 | 20 | 0 | C | PANC10 | 197.1584 | 2083.021 | 291.4515 | 269.164 | 444.0349 | 3.428841 | 402.8888 | 207.4449 |
| LNCaP_16 | 20 | 0 | C | LNCaP | 264.5144 | 3108.354 | 661.9068 | 571.2517 | 746.3527 | 8.69296 | 136.6037 | 212.3566 |
| 22RV1_27 | 20 | 0 | C | 22RV1 | 222.8237 | 2059.094 | 600.6113 | 346.3896 | 342.3383 | 53.68026 | 260.2986 | 325.1201 |
| PC3_27 | 20 | 0 | C | PC3 | 347.1039 | 2788.642 | 591.8483 | 362.8516 | 372.0377 | 3.936907 | 576.1007 | 133.8548 |
| DU145_27 | 20 | 0 | C | DU145 | 0 | 3984.754 | 664.1257 | 110.6876 | 0 | 0 | 1549.627 | 110.6876 |
| PC3_PSI_3 | 20 | 0 | C | PC3 | 514.207 | 5309.227 | 709.2236 | 547.6384 | 350.2339 | 0 | 949.6114 | 183.8728 |
| PANC10_4 | 10 | 2 | A | PANC10 | 172.5885 | 2530.92 | 493.9211 | 229.3611 | 468.9412 | 6.812705 | 420.1168 | 158.9631 |
| PC3_2 | 10 | 2 | A | PC3 | 331.7014 | 2527.471 | 597.4129 | 463.6812 | 364.4044 | 5.255832 | 1190.738 | 99.86081 |
| DU145_2 | 10 | 2 | A | DU145 | 488.5493 | 2515.863 | 1301.69 | 334.5621 | 672.4477 | 27.69554 | 2384.032 | 151.7716 |
| LNCaP_23 | 10 | 2 | A | LNCaP | 303.5862 | 2847.845 | 907.3086 | 488.1527 | 1010.804 | 20.69906 | 222.5149 | 196.641 |
| 22RV1_5 | 10 | 2 | A | 22RV1 | 235.9431 | 1506.878 | 785.8923 | 355.2303 | 416.6282 | 275.4131 | 532.4069 | 209.6298 |
| PC3_5 | 10 | 2 | A | PC3 | 339.7817 | 2495.585 | 703.4483 | 463.0585 | 459.2061 | 3.08192 | 1162.654 | 124.8178 |
| DU145_5 | 10 | 2 | A | DU145 | 426.0213 | 2630.956 | 1326.951 | 343.2115 | 786.1938 | 26.93811 | 2799.568 | 157.6378 |
| PANC10_6 | 10 | 2 | A | PANC10 | 254.5028 | 2378.719 | 462.3888 | 212.5056 | 626.5977 | 6.719546 | 646.3363 | 150.3498 |
| LNCaP_24 | 10 | 2 | A | LNCaP | 313.0846 | 2703.794 | 1015.233 | 466.352 | 994.273 | 26.19955 | 256.7556 | 192.5667 |
| 22RV1_8 | 10 | 2 | A | 22RV1 | 283.8628 | 1546.825 | 713.452 | 379.4957 | 428.8302 | 207.2047 | 531.294 | 222.3845 |
| PC3_8 | 10 | 2 | A | PC3 | 404.2422 | 2866.59 | 692.9866 | 372.1595 | 535.7813 | 1.604136 | 1111.666 | 117.904 |
| DU145_8 | 10 | 2 | A | DU145 | 435.5742 | 3022.169 | 1509.593 | 266.5157 | 802.5306 | 21.87816 | 2961.507 | 160.1083 |
| PC3_PSI_4 | 20 | 0.5 | A | PC3 | 595.1656 | 5259.403 | 772.1067 | 695.1259 | 476.8219 | 4.021389 | 2031.376 | 129.8334 |
| PC3_PSI_7 | 20 | 2 | A | PC3 | 921.611 | 2887.595 | 943.1167 | 745.0846 | 798.849 | 4.032328 | 1392.945 | 105.7366 |
| PC3_PSI_10 | 20 | 5 | A | PC3 | 863.9334 | 3103.642 | 885.5088 | 649.5568 | 779.9272 | 2.754304 | 1422.598 | 105.5817 |
| PANC10_11 | 10 | 5 | B | PANC10 | 248.803 | 2225.455 | 291.9533 | 206.5707 | 500.3602 | 1.836184 | 610.5313 | 154.2395 |
| LNCaP_2 | 10 | 5 | B | LNCaP | 315.1582 | 2425.592 | 704.885 | 606.3981 | 945.4745 | 15.47652 | 219.4851 | 215.2643 |
| 22RV1_11 | 10 | 5 | B | 22RV1 | 275.5528 | 1928.87 | 726.4574 | 335.6734 | 404.3827 | 73.0036 | 539.6541 | 249.7868 |
| DU145_11 | 10 | 5 | B | DU145 | 550.085 | 5159.985 | 964.9995 | 230.3775 | 530.1032 | 18.80632 | 1224.762 | 258.5869 |
| PANC10_14 | 10 | 5 | B | PANC10 | 227.7627 | 2120.462 | 394.1723 | 242.8908 | 531.1661 | 10.08543 | 562.2629 | 193.3041 |
| LNCaP_5 | 10 | 5 | B | LNCaP | 318.4256 | 3359.608 | 896.826 | 488.5434 | 977.0867 | 8.723989 | 189.3106 | 153.5422 |
| 22RV1_14 | 10 | 5 | B | 22RV1 | 253.7342 | 1765.887 | 685.1677 | 353.6901 | 411.7841 | 80.30644 | 498.0708 | 263.1317 |
| PC3_14 | 10 | 5 | B | PC3 | 479.9771 | 2038.191 | 673.5865 | 437.6445 | 359.2047 | 6.847922 | 1572.532 | 115.1696 |
| DU145_14 | 10 | 5 | B | DU145 | 485.8895 | 5380.687 | 964.5983 | 271.6673 | 579.2377 | 11.96772 | 1443.307 | 305.1769 |
| PANC10_17 | 10 | 5 | B | PANC10 | 218.3392 | 2764.117 | 390.7421 | 242.7251 | 570.5175 | 4.536918 | 538.1919 | 156.5237 |
| LNCaP_8 | 10 | 5 | B | LNCaP | 323.7677 | 2504.797 | 732.88 | 551.3533 | 842.6088 | 13.54676 | 199.1374 | 224.8763 |
| 22RV1_17 | 10 | 5 | B | 22RV1 | 223.4907 | 1539.308 | 752.4626 | 312.0935 | 374.2477 | 75.37851 | 518.3925 | 260.5187 |
| PC3_17 | 10 | 5 | B | PC3 | 427.3981 | 2114.419 | 707.7189 | 485.6466 | 407.0112 | 4.368635 | 1372.48 | 105.5753 |
| DU145_17 | 10 | 5 | B | DU145 | 429.5346 | 3315.436 | 1151.285 | 237.2877 | 703.0746 | 9.886987 | 2092.746 | 240.5833 |
| PC3_PSI_5 | 20 | 0.5 | B | PC3 | 617.4997 | 4780.3 | 776.2301 | 741.3868 | 489.2572 | 0.967868 | 1652.634 | 138.4051 |
| PC3_PSI_8 | 20 | 2 | B | PC3 | 831.6073 | 2816.104 | 970.0606 | 634.5776 | 678.9537 | 1.775042 | 1510.561 | 102.9524 |
| PC3_PSI_11 | 20 | 5 | B | PC3 | 789.2944 | 2774.344 | 937.4623 | 469.7323 | 840.9529 | 4.004538 | 1509.31 | 109.3239 |
| PANC10_19 | 20 | 2 | C | PANC10 | 205.5153 | 1706.844 | 345.6393 | 294.9277 | 596.528 | 2.669029 | 591.19 | 134.786 |
| PANC10_20 | 20 | 5 | C | PANC10 | 215.2123 | 1885.26 | 327.1227 | 261.3292 | 539.2605 | 4.304246 | 493.7585 | 142.655 |
| LNCaP_11 | 20 | 2 | C | LNCaP | 352.7526 | 2647.126 | 668.4513 | 628.4331 | 967.8463 | 40.01815 | 198.6086 | 154.144 |
| LNCaP_12 | 20 | 5 | C | LNCaP | 350.135 | 3013.6 | 632.3333 | 578.3324 | 989.4362 | 17.41965 | 162.0028 | 158.5188 |
| 22RV1_19 | 20 | 2 | C | 22RV1 | 227.4264 | 1484.054 | 728.5354 | 409.8815 | 459.9924 | 138.7686 | 398.3174 | 187.5946 |
| PC3_19 | 20 | 5 | C | PC3 | 372.8096 | 2153.108 | 664.3432 | 437.1237 | 400.3728 | 5.300611 | 855.872 | 114.1398 |
| PC3_22 | 20 | 2 | C | PC3 | 391.5276 | 1848.669 | 741.2415 | 496.0616 | 390.894 | 7.602477 | 947.1419 | 77.92539 |
| DU145_19 | 20 | 2 | C | DU145 | 479.4393 | 3235.696 | 935.0104 | 265.6633 | 516.7982 | 8.301979 | 1805.68 | 216.8892 |
| DU145_20 | 20 | 5 | C | DU145 | 537.9315 | 2679.769 | 654.6152 | 354.9952 | 415.3148 | 17.7992 | 962.1459 | 261.055 |
| PANC10_22 | 20 | 2 | C | PANC10 | 257.9223 | 1890.76 | 367.6788 | 237.1545 | 542.3067 | 9.490646 | 633.8635 | 165.8072 |
| PANC10_23 | 20 | 5 | C | PANC10 | 285.8775 | 2162.016 | 347.8357 | 238.0501 | 548.9282 | 7.608906 | 436.4251 | 174.4613 |
| LNCaP_14 | 20 | 2 | C | LNCaP | 278.1725 | 2702.095 | 706.0484 | 679.5053 | 829.2088 | 16.98763 | 149.7035 | 198.5429 |
| LNCaP_15 | 20 | 5 | C | LNCaP | 268.357 | 3457.212 | 646.7162 | 633.4193 | 796.6094 | 9.670523 | 151.1019 | 217.5868 |
| 22RV1_22 | 20 | 2 | C | 22RV1 | 209.413 | 1636.431 | 756.1439 | 403.7783 | 453.9371 | 125.397 | 412.5561 | 263.3337 |
| 22RV1_23 | 20 | 5 | C | 22RV1 | 212.974 | 1630.07 | 704.4524 | 362.7579 | 394.3529 | 97.1255 | 370.9492 | 270.3131 |
| PC3_20 | 20 | 5 | C | PC3 | 362.7804 | 2094.142 | 571.1295 | 421.3578 | 350.133 | 7.987825 | 983.1682 | 110.4983 |
| PC3_23 | 20 | 2 | C | PC3 | 420.0385 | 1821.143 | 588.8345 | 466.3842 | 356.1302 | 7.317744 | 1072.293 | 112.2054 |
| DU145_22 | 20 | 2 | C | DU145 | 402.6593 | 4565.827 | 1045.501 | 275.5037 | 487.4297 | 25.90206 | 2083.938 | 209.5712 |
| DU145_23 | 20 | 5 | C | DU145 | 444.1509 | 3486.85 | 1136.92 | 291.9717 | 518.4709 | 10.61715 | 1947.363 | 224.7297 |
| PANC10_26 | 20 | 5 | C | PANC10 | 220.8967 | 2337.046 | 395.3848 | 232.0429 | 567.441 | 10.13288 | 452.7369 | 172.6642 |
| LNCaP_17 | 20 | 2 | C | LNCaP | 300.7628 | 2307.117 | 713.2012 | 626.9064 | 930.2072 | 24.11178 | 214.468 | 189.0871 |
| LNCaP_18 | 20 | 5 | C | LNCaP | 315.4564 | 3161.337 | 919.2747 | 575.7563 | 941.5308 | 15.48252 | 158.6958 | 165.4694 |
| 22RV1_25 | 20 | 2 | C | 22RV1 | 255.9232 | 1339.331 | 759.2387 | 397.534 | 414.5955 | 112.6062 | 433.3632 | 288.3401 |
| 22RV1_26 | 20 | 5 | C | 22RV1 | 235.9478 | 1407.661 | 669.3212 | 390.0361 | 513.6278 | 99.51538 | 431.7683 | 258.419 |
| PC3_21 | 20 | 5 | C | PC3 | 352.4634 | 2255.47 | 633.3481 | 413.6756 | 390.9678 | 8.391986 | 894.9806 | 108.1085 |
| PC3_24 | 20 | 2 | C | PC3 | 386.1376 | 2445.213 | 680.5004 | 411.5221 | 436.9066 | 3.905311 | 845.4998 | 122.5291 |
| DU145_25 | 20 | 2 | C | DU145 | 436.3578 | 3645.017 | 950.5346 | 314.0787 | 550.5033 | 11.26617 | 1721.8 | 234.3912 |
| DU145_26 | 20 | 5 | C | DU145 | 0 | 964.7252 | 964.7252 | 241.1813 | 723.5439 | 0 | 1688.269 | 241.1813 |
| PC3_PSI_6 | 20 | 0.5 | C | PC3 | 714.2005 | 5237.471 | 603.5695 | 701.597 | 579.7628 | 0 | 1992.76 | 145.6409 |
| PC3_PSI_9 | 20 | 2 | C | PC3 | 977.4915 | 2647.156 | 907.0746 | 696.3453 | 780.8457 | 1.564821 | 1726.519 | 103.2782 |
| PC3_PSI_12 | 20 | 5 | C | PC3 | 808.5563 | 2683.619 | 886.9759 | 740.1477 | 674.075 | 1.668502 | 1572.063 | 123.4692 |
| PANC10_1 | 1 | 2 | A | PANC10 | 172.3212 | 2480.381 | 511.7418 | 224.5398 | 443.8577 | 5.221856 | 454.3014 | 208.8742 |
| LNCaP_19 | 1 | 2 | A | LNCaP | 333.6408 | 2291.777 | 799.8637 | 627.944 | 872.7111 | 39.33756 | 335.0977 | 237.4823 |
| 22RV1_1 | 1 | 2 | A | 22RV1 | 204.5698 | 1570.753 | 726.7236 | 340.4729 | 436.3203 | 178.8198 | 535.0288 | 213.1532 |
| PC3_1 | 1 | 2 | A | PC3 | 359.1507 | 2068.708 | 656.0487 | 511.5903 | 374.3149 | 8.779241 | 1169.235 | 104.5528 |
| DU145_1 | 1 | 2 | A | DU145 | 428.5803 | 2791.801 | 1272.588 | 303.6234 | 601.7662 | 29.59506 | 2236.071 | 213.7421 |
| PANC10_2 | 1 | 2 | A | PANC10 | 360.7956 | 2703.702 | 559.057 | 205.8095 | 464.4551 | 7.548024 | 968.6631 | 170.5854 |
| 22RV1_4 | 1 | 2 | A | 22RV1 | 275.6773 | 1360.843 | 883.0026 | 392.6313 | 466.1452 | 405.9974 | 636.5639 | 230.5664 |
| PC3_4 | 1 | 2 | A | PC3 | 458.1792 | 2178.244 | 964.6375 | 547.1644 | 436.4062 | 9.466512 | 1539.255 | 96.55842 |
| DU145_4 | 1 | 2 | A | DU145 | 425.2251 | 2922.966 | 1519.357 | 318.005 | 674.9992 | 13.40251 | 2932.713 | 191.2904 |
| PANC10_3 | 1 | 2 | A | PANC10 | 291.7501 | 2341.482 | 504.2041 | 230.4078 | 529.6387 | 10.47308 | 782.4888 | 227.4155 |
| LNCaP_21 | 1 | 2 | A | LNCaP | 324.7466 | 3035.675 | 943.1772 | 570.4245 | 827.398 | 45.18214 | 299.3317 | 242.854 |
| 22RV1_7 | 1 | 2 | A | 22RV1 | 311.9075 | 1321.287 | 918.4447 | 410.1174 | 428.3044 | 321.9103 | 773.8578 | 233.7033 |
| PC3_7 | 1 | 2 | A | PC3 | 497.9105 | 2513.322 | 1117.171 | 449.1203 | 439.7376 | 8.75722 | 1715.164 | 113.2183 |
| DU145_7 | 1 | 2 | A | DU145 | 463.4813 | 3105.64 | 1746.725 | 318.4463 | 838.6805 | 34.68227 | 2785.092 | 194.4309 |
| PANC10_10 | 1 | 5 | B | PANC10 | 290.1422 | 2337.876 | 388.3442 | 206.4473 | 508.8648 | 6.695589 | 594.7915 | 208.6792 |
| LNCaP_3 | 1 | 5 | B | LNCaP | 307.0182 | 3542.893 | 738.3057 | 575.05 | 833.3352 | 20.71155 | 213.2071 | 202.2422 |
| 22RV1_10 | 1 | 5 | B | 22RV1 | 256.2929 | 2413.425 | 754.2656 | 301.2566 | 372.0744 | 68.5696 | 548.5568 | 291.1398 |
| DU145_10 | 1 | 5 | B | DU145 | 515.1305 | 4297.318 | 1125.244 | 215.636 | 508.2849 | 12.83548 | 1396.5 | 266.1222 |
| PANC10_13 | 1 | 5 | B | PANC10 | 285.2892 | 2433.135 | 415.2139 | 297.1006 | 530.6016 | 9.994208 | 551.4986 | 233.501 |
| LNCaP_6 | 1 | 5 | B | LNCaP | 315.4253 | 3247.309 | 814.5465 | 559.5475 | 821.7977 | 18.12789 | 154.6913 | 212.7006 |
| 22RV1_13 | 1 | 5 | B | 22RV1 | 274.3347 | 1939.19 | 723.5315 | 313.0766 | 395.7958 | 56.54226 | 475.3738 | 288.9938 |
| PC3_13 | 1 | 5 | B | PC3 | 501.935 | 2271.85 | 918.1279 | 490.6696 | 371.1315 | 5.632687 | 2335.062 | 105.7693 |
| DU145_13 | 1 | 5 | B | DU145 | 502.594 | 4769.716 | 878.7183 | 292.3586 | 512.4488 | 16.42464 | 962.484 | 275.934 |
| PANC10_16 | 1 | 5 | B | PANC10 | 252.0552 | 2705.605 | 456.2518 | 292.4691 | 539.2067 | 13.82581 | 727.4504 | 201.006 |
| LNCaP_9 | 1 | 5 | B | LNCaP | 374.2691 | 3716.841 | 777.7604 | 548.4784 | 873.2946 | 10.11538 | 149.4829 | 173.0854 |
| 22RV1_16 | 1 | 5 | B | 22RV1 | 283.6185 | 1800.302 | 791.4308 | 341.0175 | 415.2986 | 94.53951 | 576.691 | 269.4376 |
| PC3_16 | 1 | 5 | B | PC3 | 479.2463 | 2269.692 | 984.8311 | 472.3754 | 391.0696 | 5.725762 | 1983.977 | 113.3701 |
| DU145_16 | 1 | 5 | B | DU145 | 671.7751 | 1577.681 | 628.551 | 297.1659 | 515.0876 | 9.005028 | 394.4202 | 300.7679 |

TABLE 16
Pressure-Sensitive Candidates

| | O2 | PSI | Expt | CellLine | CXorf38 | PPID | HIST1H2AG | AC112229.1 |
|---|---|---|---|---|---|---|---|---|
| LNCaP_25 | 20 | 0 | A | LNCaP | 157.323 | 796.7649 | 159.0146 | 16.91645 |
| 22RV1_3 | 20 | 0 | A | 22RV1 | 300.0304 | 661.6742 | 45.54033 | 8.036528 |
| PC3_3 | 20 | 0 | A | PC3 | 409.9796 | 470.0478 | 39.65664 | 2.915929 |
| DU145_3 | 20 | 0 | A | DU145 | 150.9284 | 185.9263 | 76.55789 | 12.03053 |
| PANC10_8 | 20 | 0 | A | PANC10 | 441.2033 | 994.5255 | 42.42339 | 67.87743 |
| LNCaP_26 | 20 | 0 | A | LNCaP | 162.3936 | 915.9727 | 149.621 | 12.77253 |
| 22RV1_6 | 20 | 0 | A | 22RV1 | 344.0181 | 634.6364 | 44.15755 | 17.45764 |
| PC3_6 | 20 | 0 | A | PC3 | 330.5891 | 599.0099 | 70.945 | 1.462784 |
| DU145_6 | 20 | 0 | A | DU145 | 146.1939 | 177.2745 | 65.61457 | 11.51133 |
| PANC10_9 | 20 | 0 | A | PANC10 | 370.1235 | 1038.943 | 35.71367 | 81.16744 |
| LNCaP_27 | 20 | 0 | A | LNCaP | 183.4312 | 896.9986 | 165.2896 | 18.14155 |
| 22RV1_9 | 20 | 0 | A | 22RV1 | 283.271 | 607.5958 | 45.15914 | 14.36882 |
| PC3_9 | 20 | 0 | A | PC3 | 358.2714 | 525.0916 | 66.61609 | 2.798996 |
| DU145_9 | 20 | 0 | A | DU145 | 127.6099 | 254.1198 | 111.1086 | 9.90077 |
| PC3_PSI_1 | 20 | 0 | A | PC3 | 346.5858 | 366.7281 | 66.67783 | 0 |
| PANC10_12 | 20 | 0 | B | PANC10 | 362.7008 | 715.6739 | 119.5106 | 31.96214 |
| LNCaP_1 | 20 | 0 | B | LNCaP | 121.4664 | 863.8873 | 160.0632 | 18.1632 |
| 22RV1_12 | 20 | 0 | B | 22RV1 | 310.9827 | 687.9044 | 60.59261 | 5.346407 |
| DU145_12 | 20 | 0 | B | DU145 | 159.7228 | 258.2752 | 147.2622 | 10.19507 |
| PANC10_15 | 20 | 0 | B | PANC10 | 363.0775 | 1083.542 | 50.07966 | 71.70497 |
| LNCaP_4 | 20 | 0 | B | LNCaP | 128.0651 | 699.6397 | 163.1145 | 16.17664 |
| 22RV1_15 | 20 | 0 | B | 22RV1 | 297.3061 | 620.931 | 59.46121 | 9.74774 |
| PC3_15 | 20 | 0 | B | PC3 | 318.9095 | 532.0582 | 42.84669 | 2.711816 |
| DU145_15 | 20 | 0 | B | DU145 | 151.2211 | 228.8479 | 112.9117 | 14.11397 |
| PANC10_18 | 20 | 0 | B | PANC10 | 350.1105 | 1150.598 | 93.69155 | 37.80536 |
| LNCaP_7 | 20 | 0 | B | LNCaP | 146.3887 | 928.5225 | 170.4383 | 9.410701 |
| 22RV1_18 | 20 | 0 | B | 22RV1 | 294.0389 | 608.622 | 60.34859 | 10.2721 |
| PC3_18 | 20 | 0 | B | PC3 | 255.0779 | 515.5685 | 71.7195 | 2.706396 |
| DU145_18 | 20 | 0 | B | DU145 | 183.5598 | 332.7586 | 141.0607 | 11.75506 |
| PC3_PSI_2 | 20 | 0 | B | PC3 | 334.4338 | 421.1631 | 76.21673 | 0 |
| PANC10_21 | 20 | 0 | C | PANC10 | 352.8687 | 1048.89 | 70.22044 | 51.67164 |
| LNCaP_10 | 20 | 0 | C | LNCaP | 141.9078 | 635.5872 | 101.9338 | 4.996755 |
| 22RV1_21 | 20 | 0 | C | 22RV1 | 353.8414 | 587.7764 | 62.3043 | 4.702211 |
| PC3_25 | 20 | 0 | C | PC3 | 279.2825 | 552.7811 | 58.66586 | 1.652559 |
| DU145_21 | 20 | 0 | C | DU145 | 148.4224 | 277.6609 | 101.9773 | 15.14514 |
| PANC10_24 | 20 | 0 | C | PANC10 | 404.2433 | 807.1225 | 49.56414 | 69.11697 |
| LNCaP_13 | 20 | 0 | C | LNCaP | 131.7968 | 669.5749 | 147.0947 | 9.414058 |
| 22RV1_24 | 20 | 0 | C | 22RV1 | 324.989 | 683.743 | 112.2689 | 10.97365 |
| PC3_26 | 20 | 0 | C | PC3 | 354.1772 | 461.9702 | 37.72757 | 3.849752 |
| DU145_24 | 20 | 0 | C | DU145 | 183.0756 | 371.0115 | 98.8284 | 11.34096 |
| PANC10_27 | 20 | 0 | C | PANC10 | 298.3092 | 756.0595 | 30.85957 | 39.43167 |
| LNCaP_16 | 20 | 0 | C | LNCaP | 136.6037 | 710.339 | 103.0737 | 3.725554 |
| 22RV1_27 | 20 | 0 | C | 22RV1 | 294.735 | 653.2787 | 100.2707 | 10.12835 |
| PC3_27 | 20 | 0 | C | PC3 | 259.1797 | 490.1449 | 72.17662 | 4.593058 |
| DU145_27 | 20 | 0 | C | DU145 | 110.6876 | 221.3752 | 221.3752 | 0 |
| PC3_PSI_3 | 20 | 0 | C | PC3 | 331.9262 | 322.3744 | 66.86283 | 0 |
| PANC10_4 | 10 | 2 | A | PANC10 | 600.6535 | 778.9193 | 39.74078 | 38.60533 |
| PC3_2 | 10 | 2 | A | PC3 | 472.4409 | 450.8336 | 30.36703 | 4.087869 |
| DU145_2 | 10 | 2 | A | DU145 | 275.8476 | 124.076 | 42.09722 | 2.215643 |
| LNCaP_23 | 10 | 2 | A | LNCaP | 162.1426 | 550.2499 | 81.0713 | 10.34953 |
| 22RV1_5 | 10 | 2 | A | 22RV1 | 380.6666 | 448.2042 | 29.82181 | 3.508448 |
| PC3_5 | 10 | 2 | A | PC3 | 372.9124 | 481.5501 | 50.85169 | 0 |
| DU145_5 | 10 | 2 | A | DU145 | 246.4338 | 149.6562 | 30.92894 | 0.997708 |
| PANC10_6 | 10 | 2 | A | PANC10 | 419.9716 | 833.6437 | 47.4568 | 29.81799 |
| LNCaP_24 | 10 | 2 | A | LNCaP | 157.1973 | 672.0185 | 58.94899 | 2.619955 |
| 22RV1_8 | 10 | 2 | A | 22RV1 | 375.7008 | 509.2833 | 34.91361 | 6.830923 |
| PC3_8 | 10 | 2 | A | PC3 | 401.836 | 542.9999 | 49.7282 | 0 |
| DU145_8 | 10 | 2 | A | DU145 | 201.8757 | 157.1249 | 94.47386 | 1.988923 |
| PC3_PSI_4 | 20 | 0.5 | A | PC3 | 328.605 | 267.7096 | 61.46981 | 0 |
| PC3_PSI_7 | 20 | 2 | A | PC3 | 481.1912 | 353.0527 | 24.19397 | 0 |
| PC3_PSI_10 | 20 | 5 | A | PC3 | 460.4279 | 359.4367 | 28.92019 | 0 |
| PANC10_11 | 10 | 5 | B | PANC10 | 454.4556 | 695.9139 | 33.05132 | 43.15033 |
| LNCaP_2 | 10 | 5 | B | LNCaP | 182.9043 | 536.0503 | 63.31302 | 14.06956 |
| 22RV1_11 | 10 | 5 | B | 22RV1 | 371.4595 | 611.9419 | 46.5219 | 5.725772 |
| DU145_11 | 10 | 5 | B | DU145 | 228.0267 | 211.5711 | 117.5395 | 9.403162 |
| PANC10_14 | 10 | 5 | B | PANC10 | 415.1836 | 770.6951 | 51.26762 | 39.50128 |
| LNCaP_5 | 10 | 5 | B | LNCaP | 153.5422 | 827.0341 | 89.85708 | 5.234393 |
| 22RV1_14 | 10 | 5 | B | 22RV1 | 334.8949 | 598.0267 | 33.31863 | 5.980267 |
| PC3_14 | 10 | 5 | B | PC3 | 423.3261 | 421.4585 | 32.99454 | 3.112692 |
| DU145_14 | 10 | 5 | B | DU145 | 209.4351 | 163.9578 | 111.2998 | 9.574176 |
| PANC10_17 | 10 | 5 | B | PANC10 | 421.9334 | 943.679 | 47.07053 | 26.08728 |
| LNCaP_8 | 10 | 5 | B | LNCaP | 146.3051 | 621.7965 | 86.69929 | 10.83741 |
| 22RV1_17 | 10 | 5 | B | 22RV1 | 363.6682 | 566 | 31.73832 | 6.61215 |
| PC3_17 | 10 | 5 | B | PC3 | 284.6894 | 405.555 | 58.97657 | 0.728106 |
| DU145_17 | 10 | 5 | B | DU145 | 147.2062 | 210.9224 | 147.2062 | 6.591324 |
| PC3_PSI_5 | 20 | 0.5 | B | PC3 | 335.8502 | 326.6554 | 55.16847 | 0 |
| PC3_PSI_8 | 20 | 2 | B | PC3 | 469.4987 | 316.845 | 31.06324 | 0 |
| PC3_PSI_11 | 20 | 5 | B | PC3 | 539.0108 | 343.1889 | 28.03176 | 0.400454 |
| PANC10_19 | 20 | 2 | C | PANC10 | 483.0943 | 533.8059 | 40.03544 | 20.01772 |
| PANC10_20 | 20 | 5 | C | PANC10 | 452.5607 | 662.2389 | 43.04246 | 30.74461 |
| LNCaP_11 | 20 | 2 | C | LNCaP | 240.1089 | 422.4138 | 59.28615 | 4.446461 |
| LNCaP_12 | 20 | 5 | C | LNCaP | 158.5188 | 505.1699 | 74.9045 | 5.225895 |
| 22RV1_19 | 20 | 2 | C | 22RV1 | 442.0038 | 444.5736 | 50.1109 | 2.56979 |
| PC3_19 | 20 | 5 | C | PC3 | 365.3888 | 425.8157 | 43.11164 | 2.826993 |
| PC3_22 | 20 | 2 | C | PC3 | 442.2107 | 392.7946 | 48.14902 | 3.167699 |
| DU145_19 | 20 | 2 | C | DU145 | 178.4925 | 199.2475 | 75.75556 | 4.150989 |
| DU145_20 | 20 | 5 | C | DU145 | 176.0144 | 205.6797 | 74.16335 | 0.988845 |
| PANC10_22 | 20 | 2 | C | PANC10 | 467.6097 | 684.5547 | 33.94302 | 30.48172 |
| PANC10_23 | 20 | 5 | C | PANC10 | 519.5796 | 717.4112 | 30.97912 | 13.58733 |
| LNCaP_14 | 20 | 2 | C | LNCaP | 160.3208 | 605.1844 | 91.30852 | 6.370362 |
| LNCaP_15 | 20 | 5 | C | LNCaP | 130.5521 | 777.2683 | 126.9256 | 6.044077 |
| 22RV1_22 | 20 | 2 | C | 22RV1 | 321.0163 | 484.0324 | 68.96835 | 3.76191 |
| 22RV1_23 | 20 | 5 | C | 22RV1 | 349.8858 | 559.3492 | 65.53046 | 1.170187 |
| PC3_20 | 20 | 5 | C | PC3 | 364.1117 | 422.6891 | 29.95435 | 1.331304 |
| PC3_23 | 20 | 2 | C | PC3 | 389.304 | 441.5039 | 35.12517 | 2.439248 |
| DU145_22 | 20 | 2 | C | DU145 | 191.9107 | 206.0391 | 70.64198 | 9.418931 |
| DU145_23 | 20 | 5 | C | DU145 | 167.2201 | 223.845 | 98.20866 | 11.50191 |
| PANC10_26 | 20 | 5 | C | PANC10 | 404.7071 | 856.228 | 55.93347 | 14.99666 |
| LNCaP_17 | 20 | 2 | C | LNCaP | 148.4778 | 553.302 | 60.91398 | 7.614247 |
| LNCaP_18 | 20 | 5 | C | LNCaP | 188.6932 | 736.3874 | 87.08918 | 7.741261 |
| 22RV1_25 | 20 | 2 | C | 22RV1 | 365.117 | 479.4294 | 54.59694 | 3.412309 |
| 22RV1_26 | 20 | 5 | C | 22RV1 | 346.6987 | 537.7041 | 64.20347 | 1.605087 |
| PC3_21 | 20 | 5 | C | PC3 | 274.961 | 448.2308 | 56.76932 | 0.493646 |
| PC3_24 | 20 | 2 | C | PC3 | 313.4012 | 483.7704 | 50.76904 | 0.976328 |
| DU145_25 | 20 | 2 | C | DU145 | 164.7058 | 204.7694 | 73.14764 | 4.451509 |
| DU145_26 | 20 | 5 | C | DU145 | 0 | 241.1813 | 0 | 0 |
| PC3_PSI_6 | 20 | 0.5 | C | PC3 | 270.2759 | 310.8873 | 47.61337 | 0 |
| PC3_PSI_9 | 20 | 2 | C | PC3 | 498.1347 | 301.4888 | 28.16678 | 0 |
| PC3_PSI_12 | 20 | 5 | C | PC3 | 464.5111 | 288.9846 | 37.37445 | 1.001101 |
| PANC10_1 | 1 | 2 | A | PANC10 | 579.626 | 704.9505 | 46.9967 | 62.66227 |
| LNCaP_19 | 1 | 2 | A | LNCaP | 166.0919 | 463.309 | 75.76123 | 8.74168 |
| 22RV1_1 | 1 | 2 | A | 22RV1 | 376.2368 | 414.8619 | 38.62507 | 2.861117 |
| PC3_1 | 1 | 2 | A | PC3 | 580.228 | 418.2111 | 35.11696 | 3.990564 |
| DU145_1 | 1 | 2 | A | DU145 | 178.6665 | 160.0325 | 46.03676 | 2.192227 |
| PANC10_2 | 1 | 2 | A | PANC10 | 587.7395 | 823.2379 | 30.1921 | 19.12166 |
| 22RV1_4 | 1 | 2 | A | 22RV1 | 435.2359 | 399.3143 | 36.75697 | 0.835386 |
| PC3_4 | 1 | 2 | A | PC3 | 383.3937 | 441.1394 | 46.38591 | 0.946651 |
| DU145_4 | 1 | 2 | A | DU145 | 218.0954 | 155.9565 | 42.64435 | 3.65523 |
| PANC10_3 | 1 | 2 | A | PANC10 | 408.4502 | 715.1619 | 91.26543 | 19.45001 |
| LNCaP_21 | 1 | 2 | A | LNCaP | 200.4958 | 553.4812 | 70.5971 | 2.823884 |
| 22RV1_7 | 1 | 2 | A | 22RV1 | 361.0124 | 435.5792 | 35.4647 | 1.818702 |
| PC3_7 | 1 | 2 | A | PC3 | 437.861 | 455.3754 | 41.28404 | 1.251031 |
| DU145_7 | 1 | 2 | A | DU145 | 223.8583 | 146.0859 | 98.79193 | 3.152934 |
| PANC10_10 | 1 | 5 | B | PANC10 | 398.3875 | 639.4287 | 20.08677 | 49.10099 |
| LNCaP_3 | 1 | 5 | B | LNCaP | 170.5657 | 650.5862 | 102.3394 | 13.40159 |
| 22RV1_10 | 1 | 5 | B | 22RV1 | 338.3516 | 611.506 | 69.69369 | 6.744551 |
| DU145_10 | 1 | 5 | B | DU145 | 158.3042 | 209.6461 | 89.84835 | 8.556986 |
| PANC10_13 | 1 | 5 | B | PANC10 | 421.5739 | 652.3492 | 69.05089 | 19.98842 |
| LNCaP_6 | 1 | 5 | B | LNCaP | 160.734 | 729.9497 | 102.7247 | 14.50231 |
| 22RV1_13 | 1 | 5 | B | 22RV1 | 362.2893 | 560.1872 | 41.88315 | 3.141237 |
| PC3_13 | 1 | 5 | B | PC3 | 443.7305 | 362.9954 | 26.91172 | 1.251708 |
| DU145_13 | 1 | 5 | B | DU145 | 225.0176 | 197.0957 | 80.48074 | 4.927392 |
| PANC10_16 | 1 | 5 | B | PANC10 | 424.3461 | 656.1943 | 72.31963 | 22.334 |
| LNCaP_9 | 1 | 5 | B | LNCaP | 206.8033 | 877.7903 | 107.8974 | 8.99145 |
| 22RV1_16 | 1 | 5 | B | 22RV1 | 360.6007 | 596.2742 | 47.26976 | 2.701129 |
| PC3_16 | 1 | 5 | B | PC3 | 352.1344 | 346.4086 | 57.25762 | 2.290305 |
| DU145_16 | 1 | 5 | B | DU145 | 203.5136 | 72.04023 | 102.6573 | 0 |

### Example 7: Primary Tumor Culture

To culture primary tumors using a method disclosed herein, target cells are isolated from a patient tumor. The cells are enriched for, for example, T-cells, dendritic cells, macrophages, B-cells, neutrophils, cancer cells, cancer stem cells, fibroblasts, and endothelial cells. The isolated cells are then co-cultured to re-establish tumor heterogeneity. To replicate the metastatic microenvironment, the cells are grown under low oxygen and high pressure conditions in an ex vivo setting. The cells are then subcutaneously injected into mice and downstream molecular assays are performed to determine gene expression changes.

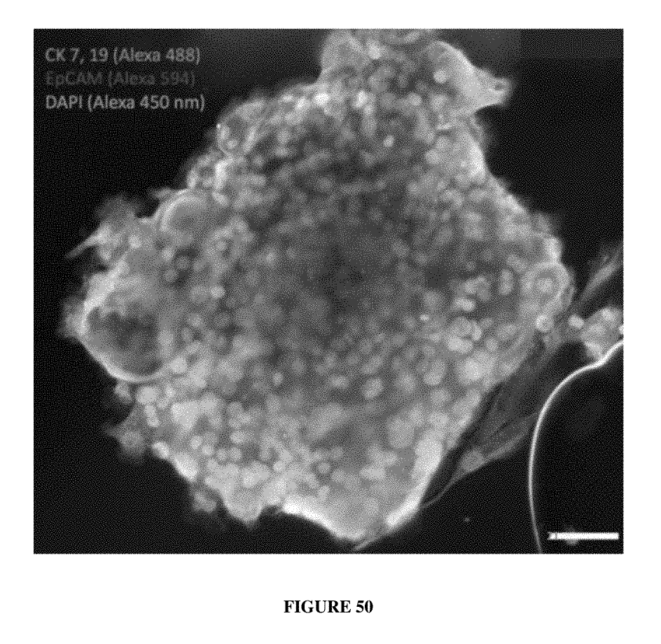

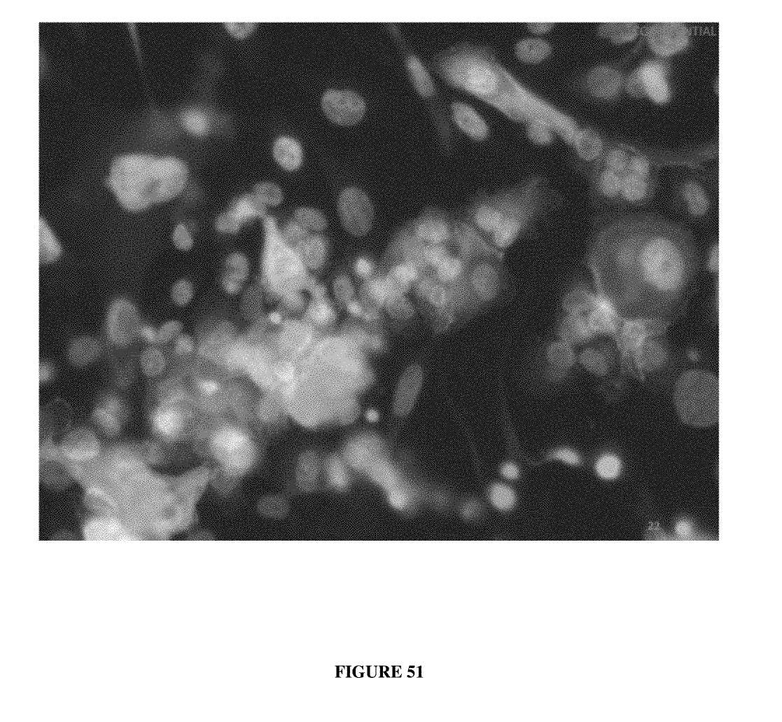

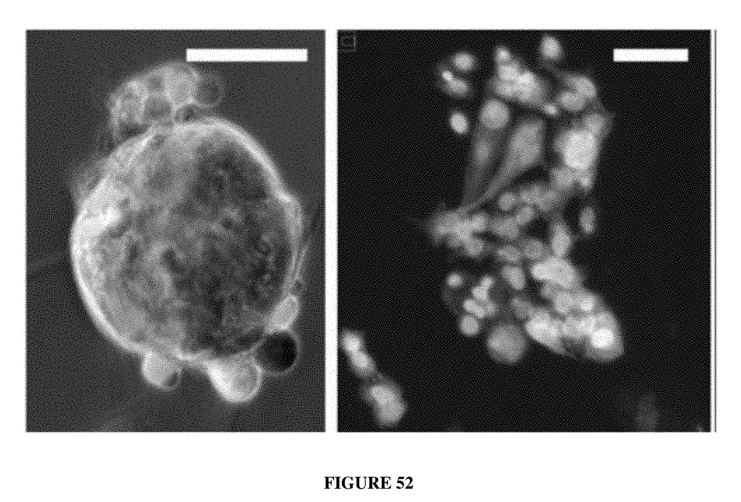

FIGS. 50-52 shows ex vivo cultures of pancreatic ductal adenocarcinoma colonies from a fine-needle aspirate. The cells were stained for DAPI. The cancerous cells were further stained for EpCAM (around periphery of cell) and CK7. The cells that did not get labeled with either CK7 or EpCAM represent the stromal cells derived from the biopsy.

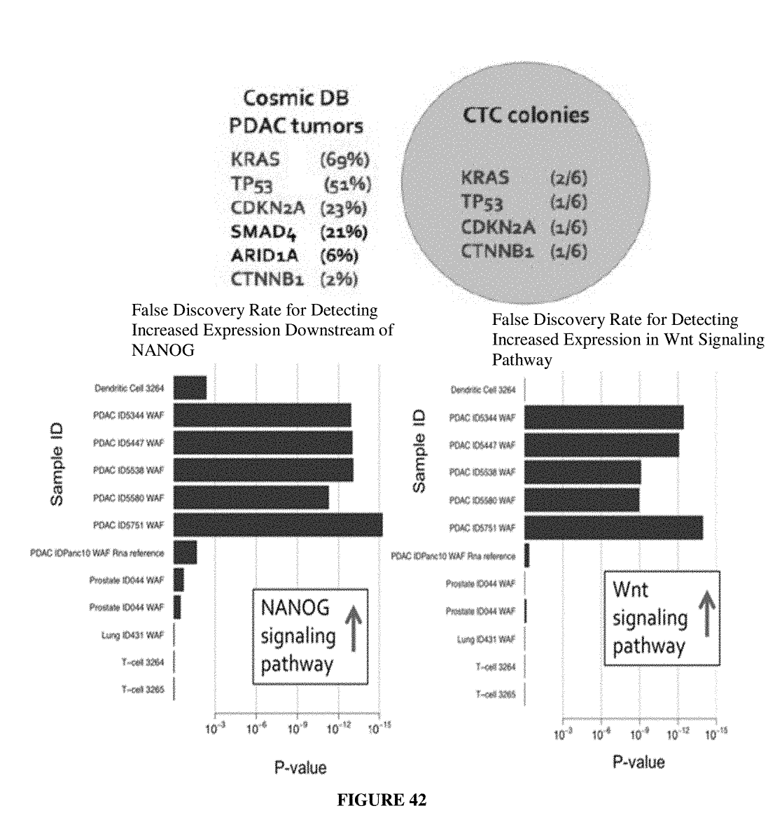

FIG. 42 shows the mutations found using the COSMIC database from pancreatic ductal adenocarcinoma (PDAC) and circulating tumor cells (CTC) cultured under low oxygen (1% O₂) and positive pressure (2 PSI) using whole exome sequencing (top panels). The bottom panels of FIG. 42 show that the NANOG and Wnt signaling pathways exhibited increased gene expression in both PDAC and CTC colonies as determined by mRNA sequencing of the cells.

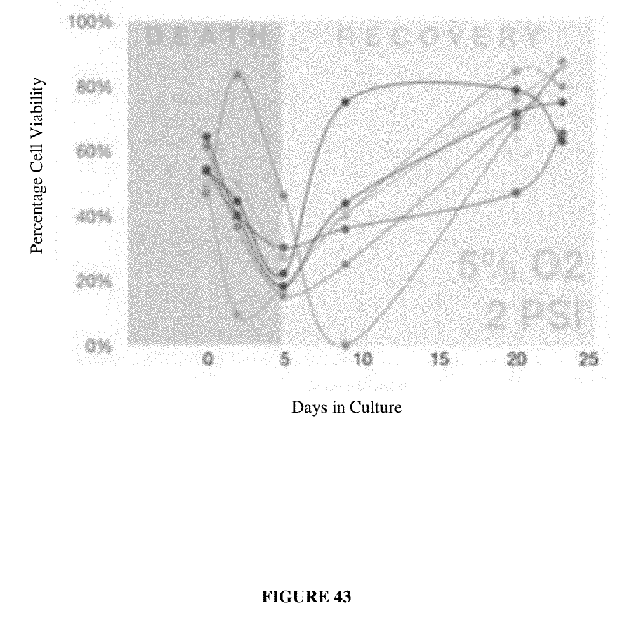

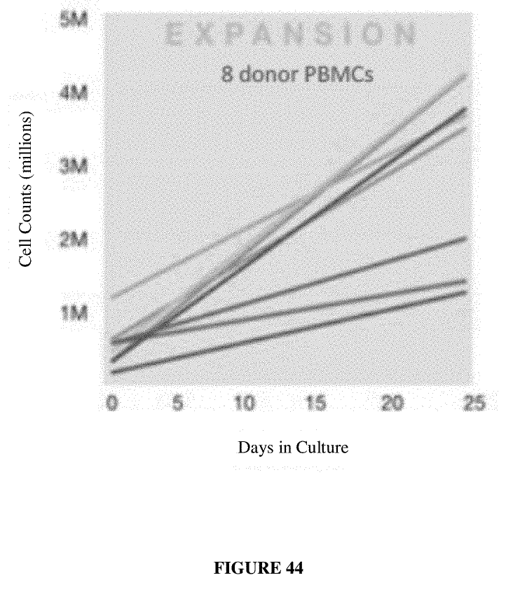

FIG. 43 shows that there was increased ex vivo expansion of primary cells. The individual lines represent patient PBMC populations from cryopreserved blood samples. The viability of individual patient PBMC populations was tracked following transfection and subsequent recovery and expansion under low oxygen and positive pressure conditions. FIG. 44 shows that there was increased ex vivo expansion of primary cells. The individual lines represent patient PBMC populations from cryopreserved blood samples. The viability of individual patient PBMC populations was tracked following transfection and subsequent recovery and expansion under low oxygen and positive pressure conditions.

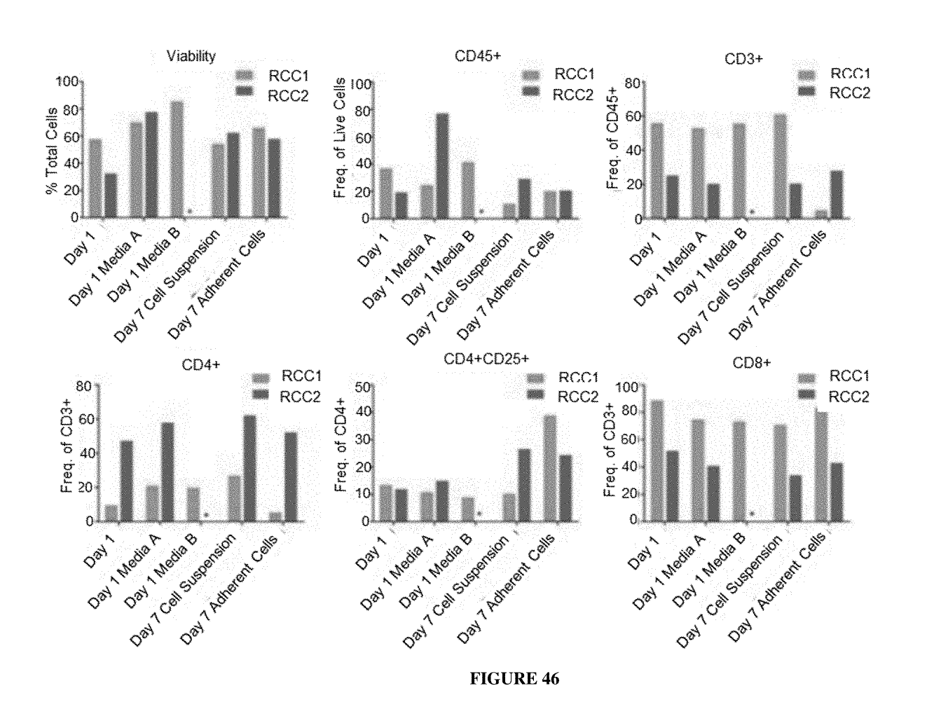

FIG. 46 shows the results of the ex vivo culture and expansion of tumor-infiltrating lymphocytes (TILs) enriched from renal cell carcinoma tumors using positive pressure and low oxygen conditions. The experiments were performed in duplicate; RCC1 and RCC2 indicate the two different cell populations analyzed. For each data point, the left bar is RCC1 and the right bar is RCC2. The results indicate that 1% O₂ and 2 PSI maintained the immune cell viability of CD3+, CD4+, CD8+, and CD11b+ cell types, as indicated by FACS analysis. The two different tumors were additionally cultured in two different culture media formulations: Media A and Media B. Media A was supplemented with 10% fetal calf serum, while Media B was animal-component free, chemically defined, and composed of recombinant human growth factors.

### Example 8: Three-Dimensional Cell Culturing Using a Method Disclosed Herein

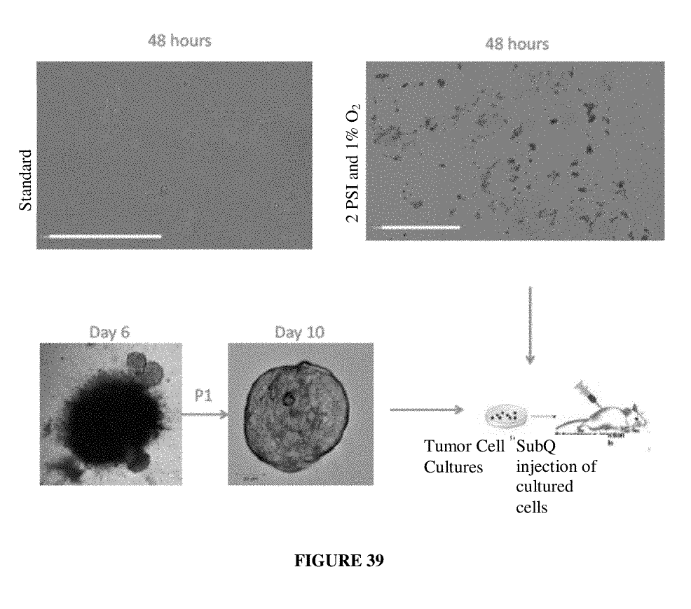

FIG. 39 shows a biopsy culture taken from a patient having prostate cancer. The cells were cultured for 48 hours under either 21% oxygen and 0 PSI or 1% oxygen and 2 PSI. The results indicated that the cells had a two-fold increase in viable cell adherence under positive pressure and low oxygen conditions. The bottom panels of FIG. 39 show that only cells grown under high pressure and low oxygen yielded enough cells for passaging at day 6, and were then able to form organoids by day 10. The tumor cell cultures were then subcutaneously injected into mice.

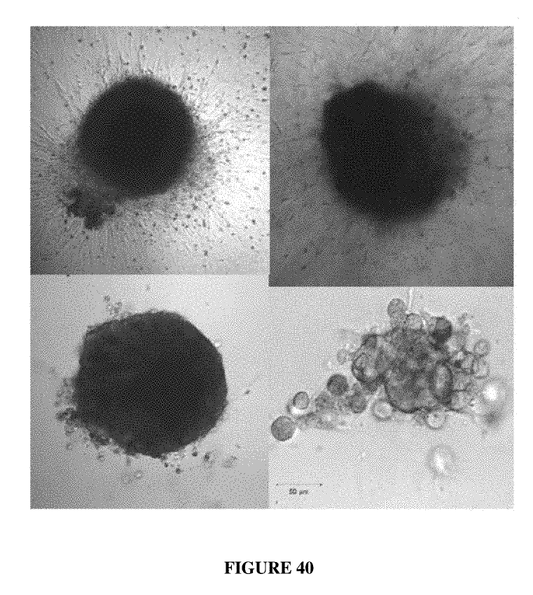

FIG. 40 shows prostate cancer cells obtained from another patient could form organoids after two weeks of culture under high pressure and low oxygen conditions.

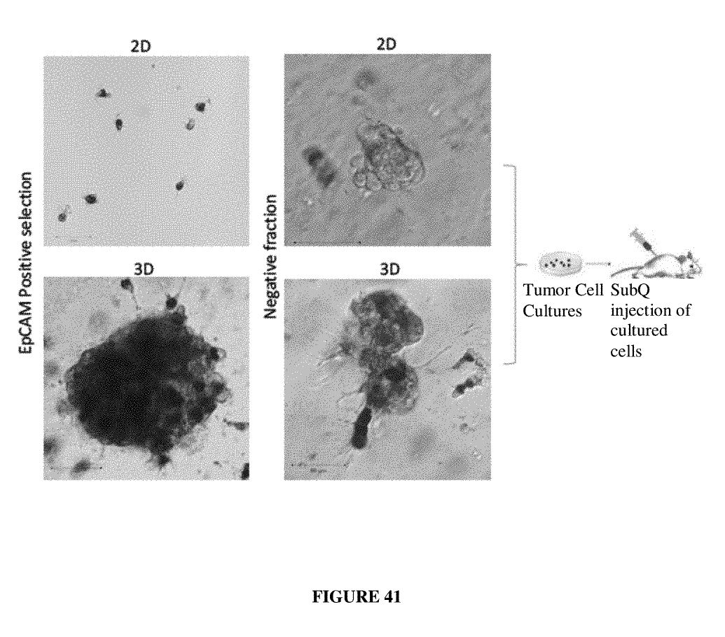

FIG. 41 shows an apheresis culture taken from a patient having prostate cancer. The cells were cultured for 10 days under high pressure and low oxygen conditions to form organoids FIG. 41 shows the results of culturing under both 2D and 3D conditions. The left panels of FIG. 41 show viable and proliferating tumor cells that were positively selected for EpCAM (prostate cancer marker), and the right panels show viable and proliferating tumor cells that are EpCAM negative. After ten days in culture, the cells were subcutaneously injected into mice.

### EMBODIMENTS

The following non-limiting embodiments provide illustrative examples of the invention, but do not limit the scope of the invention.

### Embodiment 1

A method for increasing transfection efficiency of a nucleic acid that is introduced into a cell, the method comprising culturing the cell in a hypoxic condition and a positive pressure condition, wherein culturing the cell in the hypoxic condition and the positive pressure condition increases expression of a polypeptide encoded by the nucleic acid that is introduced into the cell as compared to expression of the polypeptide encoded by a nucleic acid that is introduced into a cell that is cultured in the absence of the hypoxic condition and the positive pressure condition.

### Embodiment 2

The method of embodiment 1, wherein the cell is cultured in a culture medium that does not contain serum.

### Embodiment 3

The method of any one of embodiments 1-2, wherein the cell is contacted with a substrate.

### Embodiment 4

The method of any one of embodiments 1-3, wherein the substrate does not contain serum.

### Embodiment 5

The method of any one of embodiments 1-4, wherein the hypoxic condition comprises an oxygen level of about 2%.

### Embodiment 6

The method of any one of embodiments 1-4, wherein the hypoxic condition comprises an oxygen level of about 5%.

### Embodiment 7

The method of any one of embodiments 1-6, wherein the positive pressure condition comprises a pressure level from about 2 PSI to about 10 PSI.

### Embodiment 8

The method of any one of embodiments 1-7, wherein the nucleic acid is DNA.

### Embodiment 9

The method of any one of embodiments 1-7, wherein the nucleic acid is RNA.

### Embodiment 10

The method of any one of embodiments 1-7, wherein the nucleic acid is circular DNA.

### Embodiment 11

The method of any one of embodiments 1-7, wherein the nucleic acid is supercoiled DNA.

### Embodiment 12

The method of any one of embodiments 1-11, wherein the nucleic acid that is introduced into the cell is introduced via electroporation of the cell.

### Embodiment 13

The method of any one of embodiments 1-11, wherein the nucleic acid that is introduced into the cell is introduced via encapsulation of the nucleic acid in a cationic liposome.

### Embodiment 14

The method of any one of embodiments 1-13, wherein culturing the cell in the hypoxic condition and the positive pressure condition increases an entry rate of the nucleic acid into the cell as compared to the entry rate of the nucleic acid that is introduced into the cell that is cultured in the absence of the hypoxic condition and the positive pressure condition.

### Embodiment 15

The method of any one of embodiments 1-14, wherein the positive pressure condition is applied continuously to the cell.

### Embodiment 16

The method of any one of embodiments 1-14, wherein the positive pressure condition is applied in pulses of positive pressure to the cell.

### Embodiment 17

The method of any one of embodiments 1-16, wherein the culturing of the cell in the hypoxic condition and the positive pressure condition occurs after the nucleic acid is introduced into the cell.

### Embodiment 18

The method of any one of embodiments 1-16, wherein the culturing of the cell in the hypoxic condition and the positive pressure condition occurs before the nucleic acid is introduced into the cell.

### Embodiment 19

The method of any one of embodiments 1-16, wherein the culturing of the cell in the hypoxic condition and the positive pressure condition occurs before the nucleic acid is introduced into the cell and after the nucleic acid is introduced into the cell.

### Embodiment 20

The method of any one of embodiments 1-19, wherein the nucleic acid is introduced into the cell in the absence of the hypoxic condition and the positive pressure condition.

### Embodiment 21

The method of any one of embodiments 1-20, wherein the cell is a mammalian cell.

### Embodiment 22

A method for reprogramming a cell, the method comprising culturing the cell in a hypoxic condition and a positive pressure condition, wherein the cell exhibits a rate of reprogramming that is higher than the rate of reprogramming of a cell cultured in the absence of the hypoxic condition and the positive pressure condition.

### Embodiment 23

The method of embodiment 22, wherein the hypoxic condition comprises an oxygen level of about 2%.

### Embodiment 24

The method of embodiment 22, wherein the hypoxic condition comprises an oxygen level of about 5%.

### Embodiment 25

The method of any one of embodiments 22-24, wherein the positive pressure condition comprises a pressure level of about 2 PSI to about 10 PSI.

### Embodiment 26

The method of any one of embodiments 22-25, wherein the rate of reprogramming of the cell cultured in the hypoxic condition and the positive pressure condition is about 10% higher than the rate of reprogramming of the cell cultured in the absence of the hypoxic condition and the positive pressure condition.

### Embodiment 27

The method of any one of embodiments 22-25, wherein the rate of reprogramming of the cell cultured in the hypoxic condition and the positive pressure condition is about 20% higher than the rate of reprogramming of the cell cultured in the absence of the hypoxic condition and the positive pressure condition.

### Embodiment 28

The method of any one of embodiments 22-27, wherein the cell is a somatic cell.

### Embodiment 29

The method of any one of embodiments 22-27, wherein the cell is a fibroblast.

### Embodiment 30

The method of any one of embodiments 22-29, wherein the cell is reprogrammed into a stem cell.

### Embodiment 31

The method of any one of embodiments 22-30, wherein the cell is reprogrammed into a pluripotent stem cell.

### Embodiment 32

The method of any one of embodiments 22-29, wherein the cell is reprogrammed into an immune cell.

### Embodiment 33

The method of any one of embodiments 22-32, wherein the cell cultured in the hypoxic condition and the positive pressure condition exhibits a greater expression level of a stem cell marker as compared to the expression level of the stem cell marker for a cell cultured in the absence of the hypoxic condition and the positive pressure condition.

### Embodiment 34

The method of embodiment 33, wherein the stem cell marker is Oct4.

### Embodiment 35

The method of embodiment 33, wherein the stem cell marker is Nanog.

### Embodiment 36

The method of embodiment 33, wherein the stem cell marker is Sox2.

### Embodiment 37

The method of any one of embodiments 22-36, wherein the cell is contacted with a substrate.

### Embodiment 38

The method of any one of embodiments 22-37, wherein a nucleic acid encoding a reprogramming factor polypeptide is introduced into the cell.

What is claimed is:

1. A method for increasing transfection efficiency of a nucleic acid that is introduced into a cell, the method comprising culturing the cell in a hypoxic condition and a positive pressure condition, wherein culturing the cell in the hypoxic condition and the positive pressure condition increases expression of a polypeptide encoded by the nucleic acid that is introduced into the cell as compared to expression of the polypeptide encoded by a nucleic acid that is introduced into a cell that is cultured in the absence of the hypoxic condition and the positive pressure condition.

2. The method of claim 1, wherein the cell is cultured in a culture medium that does not contain serum.

3. The method of claim 1, wherein the cell is contacted with a substrate.

4. The method of claim 3, wherein the substrate does not contain serum.

5. The method of claim 1, wherein the hypoxic condition comprises an oxygen level of about 2%.

6. The method of claim 1, wherein the hypoxic condition comprises an oxygen level of about 5%.

7. The method of claim 1, wherein the positive pressure condition comprises a pressure level from about 2 PSI to about 10 PSI.

8. The method of claim 1, wherein the nucleic acid is DNA.

9. The method of claim 1, wherein the nucleic acid is RNA.

10. The method of claim 1, wherein the nucleic acid is circular DNA.

11. The method of claim 1, wherein the nucleic acid is supercoiled DNA.

12. The method of claim 1, wherein the nucleic acid that is introduced into the cell is introduced via electroporation of the cell.

13. The method of claim 1, wherein the nucleic acid that is introduced into the cell is introduced via encapsulation of the nucleic acid in a cationic liposome.

14. The method of claim 1, wherein culturing the cell in the hypoxic condition and the positive pressure condition increases an entry rate of the nucleic acid into the cell as compared to the entry rate of the nucleic acid that is introduced into the cell that is cultured in the absence of the hypoxic condition and the positive pressure condition.

15. The method of claim 1, wherein the positive pressure condition is applied continuously to the cell.

16. The method of claim 1, wherein the positive pressure condition is applied in pulses of positive pressure to the cell.

17. The method of claim 1, wherein the culturing of the cell in the hypoxic condition and the positive pressure condition occurs after the nucleic acid is introduced into the cell.

18. The method of claim 1, wherein the culturing of the cell in the hypoxic condition and the positive pressure condition occurs before the nucleic acid is introduced into the cell.

19. The method of claim 1, wherein the culturing of the cell in the hypoxic condition and the positive pressure condition occurs before the nucleic acid is introduced into the cell and after the nucleic acid is introduced into the cell.

20. The method of claim 1, wherein the nucleic acid is introduced into the cell in the absence of the hypoxic condition and the positive pressure condition.

21. The method of claim 1, wherein the cell is a mammalian cell.

22. A method for reprogramming a cell, the method comprising culturing the cell in a hypoxic condition and a positive pressure condition, wherein the cell exhibits a rate of reprogramming that is higher than the rate of reprogramming of a cell cultured in the absence of the hypoxic condition and the positive pressure condition.

23. The method of claim 22, wherein the hypoxic condition comprises an oxygen level of about 2%.

24. The method of claim 22, wherein the hypoxic condition comprises an oxygen level of about 5%.

25. The method of claim 22, wherein the positive pressure condition comprises a pressure level of about 2 PSI to about 10 PSI.

26. The method of claim 22, wherein the rate of reprogramming of the cell cultured in the hypoxic condition and the positive pressure condition is about 10% higher than the rate of reprogramming of the cell cultured in the absence of the hypoxic condition and the positive pressure condition.

27. The method of claim 22, wherein the rate of reprogramming of the cell cultured in the hypoxic condition and the positive pressure condition is about 20% higher than the rate of reprogramming of the cell cultured in the absence of the hypoxic condition and the positive pressure condition.

28. The method of claim 22, wherein the cell is a somatic cell.

29. The method of claim 22, wherein the cell is a fibroblast.

30. The method of claim 22, wherein the cell is reprogrammed into a stem cell.

31. The method of claim 22, wherein the cell is reprogrammed into a pluripotent stem cell.

32. The method of claim 22, wherein the cell is reprogrammed into an immune cell.

33. The method of claim 22, wherein the cell cultured in the hypoxic condition and the positive pressure condition exhibits a greater expression level of a stem cell marker as compared to the expression level of the stem cell marker for a cell cultured in the absence of the hypoxic condition and the positive pressure condition.

34. The method of claim 33, wherein the stem cell marker is Oct4.

35. The method of claim 33, wherein the stem cell marker is Nanog.

36. The method of claim 33, wherein the stem cell marker is Sox2.

37. The method of claim 22, wherein the cell is contacted with a substrate.

38. The method of claim 22, wherein a nucleic acid encoding a reprogramming factor polypeptide is introduced into the cell.


SEQUENCE LISTING (2 sequences)

SEQ ID NO: 1 - DNA, 79 bp, Homo sapiens
gagcctctgggagcctcccggctgcccacagcctctgcccctgctgggggcaggtgcctggcctctgccttcccggccc

SEQ ID NO: 2 - DNA, 89 bp, Homo sapiens
tcaaggttttaaggttctcttaatcaattctactagctaattagccaattatttaaaaataaaagtttgaaattgccaaaaaaaaaaga